(19) United States
(12) Patent Application Publication
(10) Pub. No.: US 2024/0269292 A1
(43) Pub. Date: Aug. 15, 2024

(54) INTRAVITREAL MITOCHONDRIAL-TARGETED PEPTIDE PRODRUGS AND METHODS OF USE

(71) Applicant: Eyedea Bio, LLC, Chapel Hill, NC (US)

(72) Inventors: Andres GARCIA, Chapel Hill, NC (US); Xunpei LIU, Chapel Hill, NC (US)

(21) Appl. No.: 18/566,627

(22) PCT Filed: Jun. 1, 2022
(86) PCT No.: PCT/US2022/031728

§ 371 Date: Dec. 1, 2023

Related U.S. Application Data
(60) Provisional application No. 63/195,697, filed on Jun. 1, 2021.

(51) Int. Cl. A61K 47/54; A61K 38/00; C07K 5/11

(52) U.S. Cl. CPC A61K 47/542; C07K 5/1019; A61K 38/00

(57) ABSTRACT

Described herein are therapeutic compositions for the treatment of mitochondrial disorders, and in particular mitochondrial disorders of the eye, including age-related macular degeneration (AMD). In particular, described herein are prodrugs of mitochondrial targeted tetrapeptides that have a cleavable covalent bond (e.g., an ester bond) to a conjugation moiety, wherein the conjugation moiety of the prodrug noncovalently complexes with one or more complexation agents to form drug-complex particulates with a defined avidity, and one or more drug-complex particulates are added to and dispersed within a dispersal medium forming a multiphasic colloidal suspension that serves as an extended release drug delivery system for ocular drug delivery. This extended release drug delivery system may be injected or inserted into the eye (e.g., vitreous) to reverse and prevent mitochondrial dysfunction in the eye for one or more months without requiring retreatment.

### CLAIM OF PRIORITY

This patent application claims priority to U.S. provisional patent application No. 63/195,697, titled “INTRAVITREAL MITOCHONDRIAL-TARGETED PEPTIDE PRODRUGS AND METHODS OF USE”, and filed on Jun. 1, 2021, herein incorporated by reference in its entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

Disorders of the mitochondria, either genetic or acquired, are associated with many common and rare diseases affecting various systems throughout the body, including skeletal muscle, heart, and CNS. Of particular interest to this application is mitochondrial diseases affecting the eye, especially those affecting the retina and “back of the eye.” Mitochondria are cellular organelles that generate chemical energy as ATP (adenosine triphosphate). Mitochondrial dysfunction causes loss of ATP production and bioenergetic failure (FIG. 1B) of the RPE and cells of the neurosensory retina. However, mitochondrial dysfunction also causes increased production of superoxide and other reactive oxidants, loss of normal mitochondrial calcium regulation, abnormal interactions between mitochondria and the endoplasmic reticulum, and ultimately cell death.

Thus, drugs that specifically target mitochondrial dysfunction within the eye may have potential benefit to halt or slow disease progression, prevent associated vision loss, and perhaps even restore or improve visual acuity and visual function in ocular diseases, especially retinal and back-of-the-eye diseases.

A class of tetrapeptide small molecules, also known as Szeto-Schiller (SS) peptides and disclosed by Hazel Szeto in 2000 and 2003, have been previously identified and have been demonstrated to readily penetrate cells and mitochondria, reversing mitochondrial dysfunction (FIG. 1C). The best studied of these mitochondria-targeted tetrapeptides (“MTTs”) is elamipretide.

In in vivo preclinical models, systemic administration of elamipretide partially improved visual function in a mouse model of dry age-related macular degeneration (AMD) (FIG. 2). Similarly, systemic administration of elamipretide demonstrated partial efficacy in animal models of wet AMD and retinal vein occlusion.

In patients, phase 1/2a clinical trial (ReCLAIM-1 study, ClinicalTrials.gov Identifier NCT02848313) and Phase 2b clinical trial (ReCLAIM-2 study, ClinicalTrials.gov Identifier NCT03891875) of daily systemic administration of elamipretide by subcutaneous (SQ) injection in patients with dry AMD produced a trend toward improved low-luminance visual acuity (FIG. 3) and preservation of mitochondrial structure at the ellipsoid zone of the outer neurosensory retina. This response to systemic elamipretide was seen in less than 50% of study participants.

Collectively, preclinical and human clinical trial data indicate that systemic dosing of elamipretide by SQ administration provides only incomplete therapeutic response due to inadequate eye tissue levels of drug, in animals and humans. Thus, local ocular delivery of this class of drugs, MTTs, could be effective for the treatment of retinal diseases characterized by mitochondrial dysfunction, such as dry AMD.

What is needed is an extended release drug delivery system (“XRDDS”) for MTT such as elamipretide, for IVT and other routes of ocular administration. Unfortunately, MTT are not well suited for IVT or periocular (i.e., subconjunctival or sub-Tenon's) routes of administration in their native form, due to their small size and high aqueous solubility, are poorly compatible with currently available ocular drug delivery technologies, and have not been successfully formulated in an established drug delivery system.

Thus, it would be highly desirable to provide XRDDS formulations of small molecules such as MTT in a manner that achieves sustained release in the eye and provides continuous exposure to predictable therapeutic levels in ocular tissues for a desired duration of treatment. The compositions and methods described herein are compatible with a novel complexation-based XRDDS, for ocular use.

### SUMMARY OF THE DISCLOSURE

Described herein are compositions of matter, formulations, and methods of use, for a novel extended release drug delivery system (XRDDS) comprising: novel mitochondria-targeted tetrapeptide (MTT)-prodrug, noncovalently interacting with one or more complexation agent particulates to form MTT-prodrug-complex particulates, admixed within a hydrophobic dispersal medium, that collectively forms a stable multiphasic colloidal suspension.

Extended release drug delivery systems (XRDDS) are devices, formulations or other systems used in the design, manufacture and administration of specific drug substances in a manner that regulates the drug release kinetics optimized for a specific therapeutic goal for a particular route of administration. Described herein are XRDDS that have been optimized for MTT-prodrug in and around the eye.

MTTs are four amino acid peptides comprising aromatic amino acids alternating with cationic amino acids. Typical examples of MTTs are listed in Table 1 (including SEQ IDs No. 1-635). A useful MTT for treatment of ocular disease that can also serve to form an MTT-prodrug for formulation in the XRDDS is EY005, H-d-Arg-DMT-Lys-Phe (FIG. 4A), because it has a carboxylic acid in the fourth amino acid group, facilitating a covalent linkage to a conjugation moiety (FIG. 4B). The compositions and methods described herein are not limited to EY005; in general, the methods and compositions described herein may result in a predictable release profile with any MTT (e.g., any four amino acid peptide comprising aromatic alternating with cationic amino acids), and the biological efficacy of the native MTT for treatment of mitochondrial dysfunction and aspects of ocular disease is comparable across the class of molecules (see FIGS. 5-15). Although the durability and degree of biological efficacy of the resulting composition may vary, the release XRDDS form of the composition, and methods of compounding them and releasing them, are predictable across all MTTs.

A conjugation moiety is any chemical substance that can be covalently bound to an MTT. Certain conjugation moieties can be chosen for their ability to provide properties that the native MTT does not demonstrate, especially the ability to form reversible noncovalent complexes with complexation agents. A complex is defined herein as a noncovalent interaction between the conjugation moiety of MTT-prodrug and a complexation agent.

A complexation agent is defined herein as: a chemical substance formulated as an irregularly shaped particulate ranging in size from 1 nanometer (nm) to 1000 micrometers (m). The complexation agent typically demonstrates a measurable binding capacity of MTT-prodrug, defined as a quantity of MTT-prodrug bound to a known quantity of complexation agent, and demonstrates reversibility of drug binding, defined as a measurable unbound-bound ratio, or Kd, within a specific dispersal medium. Surprisingly, the complexation agent may also be a chemical substance not previously known or expected to form complexes with an MTT-prodrug. Binding of MTT-prodrug via the conjugation moiety to a complexation agent results in formation of MTT-prodrug-complex particulate. Certain well-known chemical substances, including additives and excipients utilized in pharmaceutical industry, when formulated as irregular particulates, demonstrate a previously unknown and unexpected property to serve as complexation agents for MTT-prodrugs. Irregular particulate formulations, not dissolved individual molecules, of magnesium stearate, lecithin, albumin, cyclodextrin, and others are examples of particulate complexation agents for MTT-prodrug, a property not previously known or expected.

A colloidal suspension as described herein is a formulation that is viscous, flowable injectable liquid that forms a stable dispersal of particulates without migration or settling of the particulates (i.e., a colloid mixture). Multiphasic colloidal suspension containing MTT-prodrug (e.g., MTT-prodrug multiphasic colloidal suspension) refers to a colloidal suspension in which the MTT-prodrug is present in at least two phases: free, unbound MTT-prodrug and MTT-prodrug bound to complexation agents (as well as drug-drug aggregates). The MTT-prodrug-complex particulate serves a reservoir for MTT-prodrug when the particulate is admixed into the dispersal medium. Thus, as used herein, an MTT-prodrug multiphasic colloidal suspension may be a viscous, flowable injectable liquid that results in stably dispersed MTT-prodrug-complex particulates without migration or settling. This MTT-prodrug multiphasic colloidal suspension may enable free MTT-prodrug to dissociate from the MTT-prodrug-complex particulates to create a free MTT-prodrug concentration in the dispersal medium, and the MTT-prodrug can freely diffuse through the multiphasic colloidal suspension system to exit the implant into the adjacent environment, where the prodrug is exposed to an ocular physiologic environment, resulting in cleavage of the conjugation moiety, releasing free MTT.

The MTT-prodrug multiphasic colloidal suspension enables a drug delivery system because the particulates are a reservoir of bound MTT-prodrug, each with a unique binding capacity and Kd (unbound-bound ratio), which in turn determines the composite amount of free MTT-prodrug in the dispersal medium. Knowledge of the Kd and the binding capacity of each MTT-prodrug-complex particulate can be used to calculate the total amount of free MTT-prodrug in the system, which may determine the rate and amount of release. The relative ratio and amounts of different MTT-prodrug-complex particulates can be adjusted as described herein to create a calculatable unbound free drug within the system. The dynamic change of unbound, free drug within the system over the life of the implant may be determined by the binding capacity and Kd of the MTT-prodrug-complex particulates within the MTT-prodrug multiphasic colloidal suspension.

As used herein, a dispersal medium is a vehicle utilized in colloid mixtures. A dispersal medium is a hydrophobic oil that, when admixed with MTT-prodrug-complex particulates, can form the MTT-prodrug multiphasic colloidal suspension. The dispersal medium may not have previously been known to form a multiphasic colloidal suspension with MTT-prodrug and the chosen complexation agents.

Formulation of the MTT-prodrug in the multiphasic colloidal suspension, which may be referred to herein as a Mito XR or MTT-prodrug multiphasic colloidal suspension (and/or may be an implant or part of an implant), can be administered by intravitreal (IVT) or periocular routes to produce sustained release of therapeutic levels of active MTT drug within ocular tissues for desired duration (1 to 12 months), for the treatment of acquired and hereditary mitochondrial diseases of the eye.

More specifically, the compositions and methods described herein include MTT-prodrugs, formed by a cleavable covalent linkage to a conjugation moiety chosen from one of five classes of chemical substances specifically chosen for their ability to form noncovalent complexes with one of six chemical substances that are not previously known to serve as complexation agents for MTT-prodrugs, when formulated as irregularly shaped particulates.

In the current compositions and methods, the MTT-prodrug multiphasic colloidal suspension is injectable through a 20-gauge through 30-gauge size needle (depending on utilization) and may provide a stable dispersion of particulates without migration or settling when exposed to an ocular physiologic environment for the duration of the implant's lifetime (1 to 12 months). An ocular physiologic environment is defined as in vitro conditions with phosphate buffered saline (or comparable aqueous solvent) at 37° C. containing enzymes and proteins normally found in vitreous (representing injection into the vitreous) or with phosphate buffered saline at 37° C. containing plasma (representing injection into various periocular tissues). Alternatively, ocular physiologic environment may represent injection of the implant in vivo into the vitreous or into periocular tissues.

The MTT-prodrug multiphasic colloidal suspension also manifests the property of biodegradability when exposed to an ocular physiologic environment wherein biodegradability occurs by dissolution of the dispersal medium. The rate of biodegradation is proportional to the degree of solubility of the dispersal medium in the ocular physiologic environment. A dispersal medium with higher solubility will enable faster biodegradation of the multiphasic colloidal suspension when exposed to an ocular physiologic environment, while a dispersal medium with lower solubility will enable slower biodegradation of the multiphasic colloidal suspension when exposed to an ocular physiologic environment. This property of the MTT-prodrug multiphasic colloidal suspension can be used along with the volume of injected implant to determine durability of the implant in an ocular physiologic environment.

In general, the mitochondrial-targeted tetrapeptides (MTTs) described herein may be covalently linked to a conjugation moiety that forms noncovalent reversible interactions with particulate complexation agents, optimizing its physicochemical properties for incorporation into the multiphasic colloidal suspension examples. Also described herein are other embodiments in which the conjugation moiety alters the physicochemical properties of the MTT, including size, charge, solubility, and physicochemical interaction with vehicles, and other properties that may facilitate formulation of MTT-prodrugs in other kinds of ophthalmic drug delivery systems.

One class of MTT-prodrugs described herein are those with covalently linked conjugation moiety specifically chosen for their capacity to form noncovalent complexes with one of five classes of complexation agents.

This class of MTT-prodrugs may be compounds of formula (I):

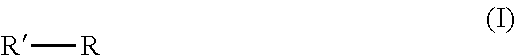

where R′ is an MTT, selected from among those with alternating cationic and aromatic amino acids listed in Table 1 (SEQ ID NOs. 1-635), in which the C-terminal amino acid in the fourth position is covalently linked by cleavable bond to conjugation moiety R, selected from among one of the following five classes of chemical substances: a C4-C30 lipid moiety, an C4-C30 straight-chain or branched aliphatic moiety, a 2-mer to 30-mer peptide moiety, a pegylated moiety, or a carbohydrate moiety.

This class of MTT-prodrugs are compounds that may be products of condensation or esterification reactions, of formula (II):

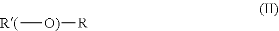

where R′ is an MTT, selected from among those with alternating cationic and aromatic amino acids listed in Table 1 (SEQ ID NOs. 1-635), in which the C-terminal amino acid in the fourth position is covalently linked by cleavable bond to conjugation moiety R, selected from among one of the following five classes of chemical substances: a C4-C30 lipid moiety, an C4-C30 straight-chain or branched aliphatic moiety, a 2-mer to 30-mer peptide moiety, a pegylated moiety, or a carbohydrate moiety.

The covalently linked conjugation moieties of MTT-prodrugs form noncovalent avid interactions (or binding) to one of six different classes of substances formulated as irregularly shaped particulates: fatty acid, organic molecules that can form keto-enol tautomers, charged phospholipid, charged protein, nucleic acid, and polysaccharides. The formation of MTT-prodrug-complex particulates optimizes the drug's physicochemical properties for compatibility with the complexation-based extended release drug delivery system (XRDDS) that is formed by admixture of one or more MTT-prodrug-complex particulates in a hydrophobic dispersal medium, enabling controlled, extended release from the stable multiphasic colloidal suspension that is specifically formulated for intravitreal (IVT) or periocular administration.

A feature of the MTT-prodrug is that the bond linking the bioactive MTT to the inactive conjugation moiety is readily cleaved by enzymatic reaction, catalysis, hydrolysis, or other chemical reaction. Upon cleavage of this bond in the MTT-prodrug, the released MTT retains full bioactivity for prevention or reversal of mitochondrial dysfunction.

The cleavable covalent bond may comprise one of: an ester bond, a hydrazone bond, an imine bond, a disulfide bond, a thioester bond, a thioether bond, a phosphate ester bond, a phosphonate ester bond, a boronate ester bond, an amide bond, a carbamate ester bond, a carboxylate ester bond, and a carbonate ester bond.

For example, from among the class of MTTs, the MTT H-d-Arg-DMT-Lys-Phe, referred to herein as EY005, can be used to form a prodrug that is a product of a condensation or esterification reaction, of formula, (II):

H-d-Arg-DMT-Lys-Phe(—O)—R, designated as EY005-R

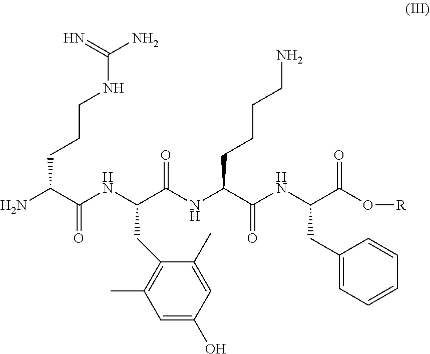

In the case of EY005 and other MTTs, R is covalently linked via ester bond at the hydroxyl group of the amino acid in the fourth position of the MTT and is selected from among one of the following five classes of chemical substances: a C4-C30 lipid moiety, an C4-C30 straight-chain or branched aliphatic moiety, a 2-mer to 30-mer peptide moiety, a pegylated moiety, or a carbohydrate moiety (FIG. 4B schematic).

One example of an EY005-prodrug includes EY005-stearyl (FIG. 16A), wherein EY005 is linked via ester bond to stearyl alcohol, one member from the group of long-chain saturated fatty alcohols. On cleavage of the ester bond, the prodrug EY005-stearyl releases the EY005 MTT. To demonstrate this experimentally, EY005-stearyl was incubated at 37° C. in vitro with carboxyesterase (0.1 g/mL), to simulate the ocular physiologic environment and the type of esterase that is readily abundant therein, within the vitreous. Incubation of EY005-stearyl with carboxyesterase produced rapid cleavage of the prodrug ester bond, releasing EY005, as evident by high performance liquid chromatography (HPLC) analysis and quantification of EY005 MTT and EY005-stearyl prodrug in solution (FIGS. 17A-17B). Upon addition of EY005-stearyl prodrug to phosphate-buffered saline solution at 37° C. without esterase, the ester bond of the EY005-stearyl prodrug cleaves more slowly (˜36 hours) by hydrolysis (FIGS. 17A and 17C). Thus, in ocular physiologic system, the covalent bond of the prodrug linking MTT to inactive conjugation is readily cleaved either by enzymatic cleavage (FIG. 18) or more slowly by hydrolysis, releasing the active MTT.

Further, upon cleavage of the covalent bond of the MTT-prodrug, the native MTT peptide retains bioactivity for treatment of mitochondrial dysfunction. For example, using an in vitro cell culture model of dry AMD (details of model and effects of MTT in cell culture model reviewed in FIGS. 5-10), EY005-stearyl (5 μM) was added to RPE cells (which possess endogenous esterases) with mitochondrial dysfunction induced by exposure to hydroquinone (HQ). EY005-stearyl effectively reversed HQ-induced mitochondrial dysfunction in RPE cells (as depicted by cellular flavoprotein-autofluorescence), with efficacy equivalent to treatment with EY005 native peptide (5 μM) (FIGS. 19A-19C). EY005-stearyl was also preincubated with carboxyesterase (0.1 μg/mL) in separate media. Recovered media containing cleaved EY005 (5 μM) was added to this RPE cellular model of mitochondrial dysfunction, and this was similarly effective and equipotent to EY005 native peptide for the reversal of RPE mitochondrial dysfunction (FIGS. 19A-19C). Thus, these studies affirm that the active MTT that is cleaved from the MTT-prodrug retains essential and unmodified bioactivity for the treatment of mitochondrial dysfunction.

In general, the conjugation moiety, R, to which the MTT is covalently linked, is not selected on the basis of bioactivity for prevention or reversal of mitochondrial dysfunction.

Also disclosed herein are MTT-prodrugs comprising homo- or hetero-dimers, trimers, multimers of any MTT, either linked together directly as a polypeptide or indirectly to a chemical substance that serves a linker moiety, which could functionally serve as a cleavable conjugation moiety.

As described herein, MTT, R′, may be covalently linked to conjugation moiety R, selected from among one of the following five classes of chemical substances: a C4-C30 lipid moiety, a C4-C30 straight-chain or branched aliphatic moiety, a 2-mer to 30-mer peptide moiety, a pegylated moiety, or a carbohydrate moiety.

One class of conjugation moieties is C4-C30 lipid moiety, with or without a preceding linker moiety that bonds the lipid moiety to the fourth amino acid of the MTT. Herein, lipid is defined as organic compounds that are insoluble in water but soluble in organic solvents. Lipids include fatty acids, fatty alcohols, glycerolipids, glycerophospholipids, sphingolipids, saccharolipids, polyketides (derived from condensation of ketoacyl subunits), sterol lipids, prenol lipids (derived from condensation of isoprene subunits), phospholipids, oils, waxes, and steroids.

One class of conjugation moieties is C4-C30 straight-chain or branched aliphatic moiety, with or without a preceding linker moiety that bonds the aliphatic hydrocarbon, to the fourth amino acid of the MTT. This class include alkanes, alkenes, and alkynes and other hydrocarbon moieties made up of 4 to about 30 carbons.

One class of conjugation moieties is peptide moiety, with or without a preceding linker moiety that bonds the peptide to the fourth amino acid of the MTT, wherein the peptide moiety comprises a natural or synthetic amino acid polymer or polypeptide chain with length of 2-mer to 30-mer, which may be anionic, cationic, or neutral in charge and contain homogeneous or heterogeneous amino acid repeats.

Anionic peptide moiety may include at least one of: poly-glutamate, poly-aspartate or a combination of glutamate and aspartate.

Cationic peptide moiety may include at least one of: poly-arginine, poly-lysine, poly-histidine, a combination of arginine and lysine, a combination of arginine and histidine, a combination of histidine and lysine, a combination of arginine, histidine, and lysine.

The peptide moiety may have one or more PEGylation sites for addition of polyethylene glycol (PEG) groups or may have one or more sites for modification by addition of sugar or carbohydrate molecules, including glycosylation.

One class of conjugation moieties is pegylated compound moiety, with or without a preceding linker moiety that bonds the pegylated compound to the fourth amino acid of the MTT, including polyethylene glycol (PEG) polymers of linear, branched, Y-shaped, or multi-arm geometries, pegylated peptides or proteins, or pegylated succinates such as succinimidyl succinate.

One class of conjugation moieties is carbohydrate molecular moiety, with or without a preceding linker moiety that bonds the carbohydrate to the fourth amino acid of the MTT, including but not limited to monosaccharides or oligosaccharides of 2 to 20 sugars. The carbohydrate molecule may comprise one or more of: glucose, galactose, lactose, mannose, ribose, fucose, N-acetylgalactosamine, N-acetylglucosamine, N-acetyleneuraminic acid, or an epimer or derivative of any of these.

In some examples a conjugation moiety, which may be combine elements from two or more of these classes, may serve as as a multimeric linker moiety that is convalently linked to multiple mitochondria targeting peptides to form dimers and/or multimers. Such linkers may be capable of generating dimers or multimers of mitochondria targeting peptides may be referred to as “multimerization domains.”

AN MTT prodrug with multimerization domain may have the formula (IV):

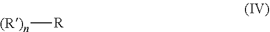

wherein R is a linker or multimerization domain which is convalently linked to multiple mitochondria targeting peptides R′, to form dimers or multimers of the prodrug and n is equal to 2 to about 100. Examples include PEG polymers, polyvinyl alcohol (PVA) polymers, or polypeptides, where the linker conjugation moiety R is covalently linked to two or more molecules of the MTT R′, to form dimers, trimers, multimers, etc. In some cases, the multimerization domains have alcohols, i.e., multiple “—OH” groups, to which the MTT units R′ are bound. In this setting, multiple MTT covalently linked (e.g., via ester or another dynamic covalent bond) to the multimerization domain may be referred to an MTT prodrug multimer.

For example, a prodrug compound may have the formula, where “n” is number comprising PVA polymer:

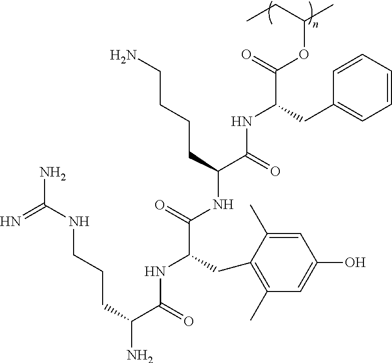

Also described hererein are multicolloidal suspensions of extended release drug delivery systems (including compositions) and methods of making and using them. The extended release drug delivery system (XRDDS) described herein is comprising MTT-prodrug admixed with one or more particulate complexation agents to form “drug-complex” particulates, which are combined and dispersed within a selected dispersal medium to form a stable multiphasic colloidal suspension.

Colloids are mixtures in which particulate substances are stably dispersed within a vehicle, called a dispersal medium, but do not settle or migrate. This differentiates a colloid from a suspension in which the particles settle within the suspension vehicle due to gravity. Typical particulate size for colloids is in the nanometer range. In colloids, the defining characteristic of the mixture is that particulates remain stably dispersed with minimal settling or migration. Colloid mixture in which particulates are dispersed in a liquid is called a “sol.” Colloid mixtures in which particulates are dispersed in a solid or semisolid is called a “solid colloid.” Colloid mixtures in which particulates are stably dispersed in a viscous semi-solid or solid dispersal medium have not been given a defined name. Herein, we refer to stably dispersed particulates as a “colloidal suspension,” in reference to the larger sized stably dispersed particulates rather than nanoparticulates present in a typical colloid. As described herein, in some examples the dispersal medium is a hydrophobic dispersal medium that facilitates a stable colloidal suspension. A multiphasic colloidal suspension is a suspension in which the drug substance is present in more than one phase, including free drug, drug-drug aggregates, and most importantly, drug noncovalently bound to complexation agent particulates. As described herein, a multiphasic colloidal suspension may incorporate an MTT-prodrug as the drug substance.

The complexation agent described herein may be noncovalently complexed with the conjugation moiety of the prodrug and incorporated and stably dispersed within a dispersal medium, which forms the multiphasic colloidal suspension.

Complexation of MTT-prodrugs to particulate complexation agents within the dispersal medium serves to limit the release of free MTT-prodrug into the dispersal medium. While the dispersal medium restricts access of water to the MTT-prodrug-complex particulates, free, unbound MTT-prodrug substance diffuses freely within the dispersal medium, and the dispersal medium does not retain the free, unbound drug, which can diffuse out of the multiphasic colloidal suspension.

Complexation occurs in two physicochemical circumstances. In one case, complexation occurs with noncovalent interactions between individual molecules (e.g., receptor-ligand interactions). This type of complexation is termed molecular complexation. The second circumstance involves a molecule of a chemical substance (in this case, molecule of drug) that noncovalently binds or adsorbs to a surface of a particulate (in this case, a complexation agent). This type of complexation is termed particulate complexation, and different particulate adsorbents, or complexation agents, have different sorptive properties based on size and shape of particulate, functional groups present at the surface, and the surface irregularity and porosity of the particulate. The utility of particulate complexation has been recognized in other disciplines, including soil sciences, wherein a chemical adsorbent (e.g., alumina, silica gel, activated charcoal) interacts with specific chemicals (frequently contaminants) in soil; the hydrocarbon industry, wherein adsorbents (e.g., polypropylene, vermiculite, perlite, polyethylene, others) are used to clean oil spills or to remove residual oil from drilling and fracking equipment; and industrial coatings (e.g., zeolite, silica gel, aluminum phosphate), wherein adsorbents are used to bind chemical substances for various purposes (i.e., lubrication, surface cooling).

In medical applications, adsorbents are used for the treatment of acute poisoning by ingestion (e.g., activated charcoal, calcium polystyrene sulfate, aluminum silicate) where the adsorbent binds the toxin to limit adsorption from the gut into systemic circulation. In the pharmaceutical industry, principles of adsorption complexation are used to understand chemistry of drug binding to plasma proteins in the blood, drug coatings on solid scaffolds for in situ drug release (e.g., drug-eluting stents), and affixing excipients to insoluble drugs in order to improve oral bioavailability and gut absorption.

The compositions and methods described herein may utilize particulate complexation, wherein complexation agents thus are chemicals compatible with ocular tissues that, when formulated as an irregularly shaped particulates, have the capacity of noncovalently binding MTT-prodrug, forming MTT-prodrug-complex particulates. One or more MTT-prodrug-complex particulates are incorporated and admixed into a hydrophobic dispersal medium to form a stable multiphasic colloidal suspension, that is safely delivered into and around the eye, to produce continuous exposure to predictable therapeutic levels of MTT-prodrug in ocular tissues for a desired duration of treatment.

The conjugation moiety of the MTT-prodrug is specifically chosen for its ability to complex, or form noncovalent interactions, with one or more particulate complexation agents to form “drug-complex” particulates, which are subsequently combined and dispersed within a selected dispersal medium to form a stable multiphasic colloidal suspension. Complexation agents are selected from one of six classes of chemical substances, including fatty acid, organic compounds that can form keto-enol tautomer, charged phospholipid, charged protein, nucleic acid, and polysaccharides.

The compositions and methods described herein discloses a new property, not previously recognized, of these six classes of chemical substances, fatty acid, organic compounds that can form keto-enol tautomer, charged phospholipid, charged protein, nucleic acid, and polysaccharides, that, when in the form of an irregularly shaped particulate with irregular surface, can serve as an effective complexation agent for MTT-prodrugs. The criteria for complexation agent may include: (1) fluorescein-labeled conjugation moiety of the MTT-prodrug binds to the particulate via the conjugation moiety and not the MTT itself, and this is demonstrable by microscopy imaging (see FIGS. 20A-20D, 21A-21D, 22A-22D, 23A-23D); (2) when particulate of substance is added to a solution of MTT-prodrug, upon centrifugation and pulldown of the particulates, pharmacologically significant quantities of drug are observed to be complexed to the particulates (see Table 2, below); (3) drug particulate-complexes, when resuspended in appropriate dispersal medium, demonstrate partial release of drug, which can be demonstrated by Kd or unbound-bound fraction of drug for a given MTT-prodrug-complexation agent pair in a particular dispersal medium (see Table 2, below); and (4) the drug-particulate complexes provide a useful pharmacokinetic release profile from the dispersal medium (see FIG. 25B). Collectively, these four properties define a complexation agent and enable the presently described complexation-based XRDDS (FIG. 26).

In contrast, spherical particulates with a spherical smooth surface and non-reactive coating, including for example silicone beads, latex beads, and certain polymeric microparticulates, fail to form complexes with MTT-prodrug, and therefore may be excluded (see, e.g., FIGS. 24A-24D).

One class of complexation agents is fatty acid, which is a carboxylic acid with an aliphatic chain, which may be either saturated or unsaturated, and may be in the form of a salt or ester. For example, the fatty acid may have a chemical formula of CH3(CH2)ₙCOOH where n is equal to between 4 and 30. Specific examples of salt form fatty acids include magnesium stearate, magnesium palmitate, calcium stearate, calcium palmitate, and others.

One class of complexation agents is organic compounds that can form keto-enol tautomers. Tautomers refer to molecules capable of undergoing chemical equilibrium between a keto form (a ketone or an aldehyde) and an enol form (an alcohol). Usually, a compound capable of undergoing keto-enol tautomerization contains a carbonyl group (C═O) in equilibrium with an enol tautomer, which contains a pair of doubly bonded carbon atoms adjacent to a hydroxyl (—OH) group, C═C—OH as depicted herein:

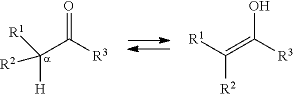

The relative concentration of the keto and enol forms is determined by the chemical properties of the specific molecule and the chemical microenvironment, including equilibrium, temperature or redox state. Organic compounds capable of keto-enol tautomerization include but are not limited to phenols, tocopherols, quinones, ribonucleic acids, and others.

One class of complexation agents is charged phospholipid. In general, phospholipids consist of a glycerol molecule, two fatty acids, and a phosphate group that is modified by an alcohol, wherein the polar head of the phospholipid is typically negatively charged. Examples include lecithin, phosphatidylcholine, phosphatidylethanolamine, phosphatidylserine, different phospholipids in oil, and many others, which may be used individually or in combination to serve as complexation agents. Anionic phospholipids may comprise one of: phosphatidic acid, phophatidyl serine, sphingomyelin or phophatidyl inositol. In some instances, synthetic, ionizable phospholipids with positive charge can manufactured, including but not limited to examples such as DLin-MC3-DMA. Additional cationic phospholipids may comprise one of: cationic triesters of phosphatidylcholine; 1,2-dimyristoylsn-glycerol-3-phosphocholine (DMPC); 1,2-dioleoyl-sn-glycerol-3-phosphocholine (DOPC); 1,2-bis(oleoyloxy)-3-(trimethylammonio)propane (DOTAP); 1,2-dioleoyl-sn-glycerol-3-phosphoethanolamine (DOPE); 1,2-dipalmitoyl-sn-glycerol-3-phosphocholine (DPPC); 1,2-dioleoyl-sn-glycerol-3-ethylphosphocholine (EDOPC);1,2-dimyristoyl-sn-glycerol-3-ethylphosphocholine (EDMPC); 1,2-dipalmitoyl-sn-glycerol-3-ethylphosphocholine (EDPPC). In pharmaceutical sciences, phospholipids have been used for drug formulation and delivery applications to improve bio-availability, reduced toxicity, and improved cellular permeability. However, in the compositions and methods described herein, phospholipids may be used as a complexation agent particulate to noncovalently bind the conjugation moiety of the MTT-prodrug and form MTT-prodrug complex particulates for the purpose of regulating free MTT-prodrug in the dispersal medium of the stable multiphasic colloidal suspension in which the MTT-prodrug complex particulates are incorporated and dispersed therein.

One class of complexation agents is charged proteins. Proteins are large biomolecules and macromolecules that comprise one or more long changes of amino acid residues. Amino acids that make up proteins may be positive, negative, neutral, or polar in nature, and collectively, the amino acids that comprise the protein give it its overall charge. A variety of proteins, based on size, molecular weight, ability to readily form particulates, and compatibility with ocular tissues could serve as complexation agents. The charge of the protein will determine its compatibility with a specific MTT-prodrug, such that negatively charged proteins will readily complex with positively charged conjugation moiety of MTT-prodrug, while positively charged proteins (e.g., Arg-Gln-Ile-Arg-Arg-Ile-Ile-Gln-Arg-NH₂ and synthetic peptides with positive charge) will readily complex with negatively charged conjugation moiety of MTT-prodrug. Examples of proteins that could serve as complexation agents include albumin and collagen.

One class of complexation agents is nucleic acids, biopolymer macromolecules comprising nucleotides, comprising a 5-carbon sugar, a phosphate group, and a nitrogenous base. The importance of nucleic acids for biologic function and encoding genetic information is well established. However, nucleic acids also have a variety of applications, including nucleic acid enzymes (e.g., carbon nanomaterials), aptamers (e.g., for formation of nucleic acid nanostructures and therapeutic molecules that function in an antibody-like fashion), and aptazymes (e.g., which can be used for in vivo imaging). In pharmaceutical sciences, specially engineered nucleic acids have been considered and applied for use in carrier-based systems in which the nucleic acid serves as a carrier system for various types of drugs. However, in the compositions and methods described herein, nucleic acids are considered not as a carrier system but rather as a complexation agent, as they are highly negatively charged and thus, formulated as a particulate, could then serve as a complexation agent for positively charged conjugation moiety of the MTT-prodrug.

One class of complexation agent is polysaccharides, long chain polymeric carbohydrates comprising monosaccharide units bound together by glycosidic linkages. Frequently, these are quite heterogenous, containing slight modifications of the repeating monosaccharide unit. Depending on structure, they can be insoluble in water. Complexation of polysaccharide particulate complexation agents to other molecules, in this case, various MTT-prodrugs, can occur through various electrostatic interactions and is influenced by charge density of conjugation moiety of MTT-prodrug and polysaccharide, ratio of polysaccharide complexation agent to MTT-prodrug, ionic strength, and other properties. Examples of polysaccharides that could serve as complexation agents include a ringed polysaccharide molecule, cyclodextrins, a clathrate, cellulose, pectins, or acidic polysaccharides (polysaccharides that contain carboxyl groups, phosphate groups, or other similarly charged groups.

In the compositions and methods described herein, the conjugation moiety of the MTT-prodrug has specific avidity for, and complexes with, a given complexation agent, forming an MTT-prodrug-complex particulate. This avidity can be measured as Kd, the unbound-bound fraction of an MTT-prodrug for a given MTT-prodrug-complex particulate in a selected dispersal medium. Additionally, MTT-prodrug-complex particulate demonstrates a measurable binding capacity of MTT-prodrug, defined as a quantity of MTT-prodrug bound to a known quantity of complexation agent. The binding of the conjugation moiety of the MTT-prodrug to a particular complexation agent thus serves to limit the free drug available for release from a given dispersal medium.

As described herein, one example of EY005-prodrugs includes EY005-stearyl (e.g., FIG. 16A). As EY005 is linked via ester bond to stearyl alcohol, the resultant EY005-stearyl prodrug is hydrophobic, as compared to the unmodified MTT EY005, which is highly hydrophilic. EY005-stearyl readily forms noncovalent complex with solid lipid particulate complexation agents, such as magnesium stearate, to form MTT-prodrug-magnesium stearate particulates. The high avidity interaction between the hydrophobic, long-chain fatty alcohol of the conjugation moiety of this MTT-prodrug and the particulate complexation agent magnesium stearate serves to bind the MTT-prodrug and limits the free, unbound MTT-prodrug that is available for release from the dispersal medium in which the MTT-prodrug-complex particulate is dispersed.

Another specific example of EY005-prodrugs includes EY005-tri-glutamate (triGlu) (FIG. 16B), wherein EY005 is linked via ester bond to glutamate trimer/tripeptide, a negatively charged peptide conjugation moiety that readily forms noncovalent complex with positively charged particulate complexation agents to form MTT-prodrug-complex particulates. The high avidity interaction between the negatively charged conjugation moiety of this and the positive charge of the particulate complexation agent serves to bind MTT-triGlu prodrug and limits the free, unbound MTT-prodrug that is available for release from the dispersal medium in which the MTT-prodrug-complex particulate is dispersed.

Another specific example of EY005-prodrugs includes EY005-tri-arginine (triArg) (FIG. 16C), wherein EY005 is linked via ester bond to arginine trimer/tripeptide, a positively charged peptide conjugation moiety that readily forms noncovalent complex with negatively charged particulate complexation agents to form MTT-prodrug-complex particulates. The high avidity interaction between the positively conjugation moiety of this and the negative charge of the particulate complexation agent serves to bind MTT-triArg prodrug and limits the free, unbound MTT-prodrug that is available for release from the dispersal medium in which the MTT-prodrug-complex particulate is dispersed.

As described herein, formation of MTT-prodrug-complex particulates can be verified experimentally by direct visualization. For example, the MTT-prodrug EY005-stearyl was fluorescently labeled with fluorescein isothiocyanate (FITC) and admixed with different complexation agents. The resultant mixture was then visualized under direct fluorescence microscopy. Using this approach, FITC-labeled EY005-stearyl was observed to form drug-complex particulates with several different complexation agents: magnesium stearate (as previously described, and as expected); albumin, a large, charged carrier protein; and cyclodextran, a large cyclic carbohydrate molecule, and lecithin, an anionic phospholipid (see FIGS. 20A-20D, 21A-21D, 22A-22D, 23A-23D). In contrast, FITC-labeled EY005-stearyl was not observed to form drug-complex particulates with silica microbeads (see FIG. 24A-24D), indicating the process of complexation and drug-complex particulate formation is highly dependent on favorable noncovalent interaction between drug and complexation agent.

Further, this noncovalent interaction is specifically mediated by the conjugation moiety of the MTT-prodrug. FITC-labeled EY005-stearyl that had been admixed with complexation agent was treated with an aqueous solution of carboxyesterase (0.1 μg/mL) to hydrolyze the ester bond of the prodrug, releasing the fluorescent peptide. Complexed particulates were no longer fluorescently labeled by microscopy, affirming that complexation of the prodrug is specifically mediated by the conjugation moiety of the MTT-prodrug (see FIGS. 20D, 21D, 22D, 23D).

As described herein, formation of drug-complex particulates in which the complexation agent has high avidity for the drug can be quantified and verified experimentally. For example, the MTT-prodrug EY005-stearyl was admixed with known quantities of selected individual complexation agents. The EY005-stearyl-complexation agent mixture was then added to an appropriate dispersal medium (in this case, methyl laurate), and centrifuged to “pull down” or separate EY005-stearyl bound to complexation agent from unbound prodrug present in the dispersal medium. HPLC analysis of pulled down particulates and dispersal medium from EY005-stearyl content determined the fraction of MTT-prodrug that is bound to the complexation agent and calculation of the Kd value, the unbound to bound coefficient, for the MTT-prodrug/complexation agent pair. Using this type of assay, Kd values can be generated to identify the unbound to bound drug ratio for specific MTT-prodrug/complexation agent pairs in a selected dispersal medium (see, e.g., Table 2, below).

In the compositions and methods described herein, the dispersal medium as defined herein is a hydrophobic liquid that stably disperses MTT-prodrug complex particulates and forms a stable multiphasic colloidal suspension upon admixture with MTT-prodrug and particulate complexation agents.

The compositions and methods described herein disclose new and previously unrecognized properties of certain oils that allow them to serve as effective dispersal medium. These include hydrophobicity, high starting viscosity, and other properties that allow it to form a stable multiphasic colloidal suspension when admixed with MTT-prodrug-complex particulates. The criteria that define a stable multiphasic colloidal suspension include uniform mixture and distribution of the MTT-prodrug-complex particulates without settling, separation, or dissociation of the particulates for the prespecified duration of the implant's lifetime, after exposure to an ocular physiologic environment in vitro (i.e., 37° C., buffered saline, vitreous enzymes, dilute serum) or in vivo when injected into the eye. The stability is also dependent on the relative percentage of MTT-prodrug-complex particulates to oil (weight to weight) and the size and mass of the particulates.

Four classes of oils that meet these criteria for formation of a stable multiphasic colloidal suspension include saturated fatty acid methyl esters, unsaturated fatty acid methyl esters, saturated fatty acid ethyl esters, or unsaturated fatty acid ethyl esters. A dispersal medium can be an individual oil from one of these classes or can be designed as a mixture of oils with different viscosity values that are specifically designed and admixed to achieve the desired goal of a stable colloidal suspension.

In contrast, certain other oils and viscous substances including silicone oil, viscous gelatin, and viscous proteoglycan fail to form a stable multiphasic colloidal suspension or rapidly decompensate when exposed to a physiologic ocular microenvironment (.e., 37° C., buffered saline, vitreous enzymes, dilute serum) or in vivo when injected into the eye.

In one example of MTT-prodrug multiphasic colloidal suspension, EY005-stearyl admixed with magnesium stearate (solid fatty acid) complexation agent and EY005-stearyl is admixed with alpha-tocopherol (keto-enol tautomer) complexation agent, and both drug-complex particulate pairs are incorporated into methyl laurate to form the stable multiphasic colloidal suspension, or Mito XR bolus implant.

In in vitro kinetics studies, this pilot formulation of Mito XR achieved zero-order (i.e., linear) kinetics of EY005 bioactive tetrapeptide, achieving the desired durability of drug release of three months, with free bioactive MTT within the dispersal medium released from the implant into the ocular physiologic environment (see FIG. 27).

In in vitro efficacy studies, bolus implant of Mito XR was added to RPE cell culture model with endogenous esterases. Cell culture data demonstrated restoration of cytoskeleton, with ˜80% improvement at 21-day timepoint (FIGS. 28A-28D) in association with reversal of cellular mitochondrial dysfunction. This data affirms that EY005-stearyl, admixed with complexation agents and incorporated into a dispersal medium to form a stable multiphasic colloidal suspension in a formulation of Mito XR, can produce sustained release of EY005 at predictable therapeutic levels, which is bioactive upon cleavage of the MTT-prodrug that is released from the dispersal medium of the multiphasic colloidal suspension into the surrounding ocular physiologic environment.

In some examples, the MTT-prodrug may be formulated within the presently described complexation-based extended release drug delivery system, Mito XR, deployed into the eye of animals or humans. For example, intravitreal administration of MTT-prodrug formulated within the complexation-based extended release drug delivery system in the eyes of rabbits has been found to produce sustained release of active MTT at the desired daily release rate and achieving desired target tissue levels of drug in the vitreous and retina.

In in vivo kinetics studies, using LC/MS analysis, we measured high retina EY005 levels (>300 ng/g) sustained through 6 weeks after IVT Mito XR (EY005-stearyl payload 1 mg) bolus injection in rabbit eyes (FIG. 29), affirming that endogenous esterases release active EY005 in vivo. Recovered bolus had ˜50% residual payload, indicating that implant formulation will achieve ˜90 day release of EY005 levels >EC₅₀, given zero-order release kinetics.

Importantly, formulation of Mito XR appeared to be well tolerated clinically in rabbit eyes (FIG. 30A), with no histologic evidence of toxicity (FIG. 30B).

In contrast to EY005-stearyl prodrug, the EY005 native tetrapeptide fails to form noncovalent interaction with complexation agent. FITC-labeled EY005 when admixed with different complexation agents (e.g., magnesium stearate, albumin, cyclodextrin, lecithin), did not produce visible drug-complex particulates (FIGS. 20B, 21B, 22B, and 23B).

Further, incorporation of EY005 native bioactive peptide with the same complexation agent and into the same dispersal medium used for Mito XR formulation of EY005-stearyl produced excessive release, or “dump” of the bioactive MTT in vitro (FIG. 27). Additionally, multiphasic colloidal suspension bolus formulation of EY005 native peptide administered into the vitreous did not produce detectable EY005 tissue levels beyond 21 days (FIG. 29), indicating excessive release of the native MTT drug in vivo as well. Moreover, no residual drug in the recovered bolus, consistent with excessive drug release or “dumping.” Thus, the incorporation of the native unmodified MTT into the multiphasic colloidal suspension is insufficient to produce sustained release and fails to achieve specifications of an extended release drug delivery system. Importantly, these data affirm and underscore the necessity for the prodrug construct and the specific interaction between prodrug conjugation moiety and complexation agent to form MTT-prodrug-complex particulates, to order to achieve controlled, durable release of the active MTT into the tissue following cleavage of covalent bond of the free MTT-prodrug released from the dispersal medium of multiphasic colloidal suspension (FIGS. 31-32).

The MTT-prodrug compounds described herein, interacting with one or more particulate complexation agents to form MTT-prodrug-complex particulates, which, when admixed in the appropriate dispersal medium to form a stable multiphasic colloidal suspension and a resultant formulation of Mito XR, may provide a vitreous and retina concentration of the active MTT that meets or exceeds the EC₅₀ (i.e., effective concentration of the drug that produces 50% maximal response for reversal of mitochondrial dysfunction), for 1 to 12 months or more duration following a single administration of the Mito XR implant.

Sustained, high ocular tissue levels, and the resultant benefits for treatment of retina disease pathobiology described herein, are not feasible with systemic administration or with intravitreal administration of the unmodified mitochondrial-targeted peptide. Successful incorporation of MTT-prodrug into a compatible XRDDS, in this case, the complexation-based XRDDS is essential to achieve these therapeutic benefits for ocular diseases (e.g., age-related macular degeneration (AMD)).

The compositions and methods of Mito XR described herein may be applied by delivery of the implant to the eye by intravitreal or periocular routes of administration to treat various retinal and back of the eye diseases, include dry AMD, wet AMD, diabetic retinopathy (DR), retinal vein occlusion (RVO), acquired and inherited retinal degenerations, and other retinal and optic nerve diseases.

Implants of Mito XR have been characterized by in vitro studies for release and cellular efficacy and by in vivo studies for toxicology, pharmacokinetics (PK), and efficacy, demonstrating their potential utility for clinical use in humans and animals affected by retinal diseases.

The release of the bioactive drug from the implant is dependent on the diffusion of free, unbound MTT-prodrug within the dispersal medium of the multiphasic colloidal suspension into the surrounding ocular physiologic environment and release of the active MTT from the prodrug by cleavage of the covalent bond either via natural enzymes within the tissue compartment of the body (i.e., within the vitreous or within periocular tissues). Alternatively, release of the active MTT from the prodrug may occur by hydrolysis of MTT-prodrug that is released from the implant into the ocular physiologic environment.

A therapeutic composition for local ocular administration may include: any of the MTT-prodrugs described herein, where the conjugation moiety of the MTT-prodrug forms noncovalent interaction (complex) with selected compatible complexation agent to form MTT-prodrug-complex particulates, which are then incorporated and admixed within a hydrophobic dispersal medium to form a stable multiphasic colloidal suspension. The combined effect of conjugation moiety, complexation, and stable dispersion of complex particulates within multiphasic colloidal suspension alters the physicochemical properties of the active MTT drug, limits the amount of free MTT-prodrug available for release from the implant into the ocular physiologic environment, and restricts access of water to MTT-prodrug-complex particulates, facilitating sustained release and continuous, predictable exposure of therapeutic levels of active drug for desired duration of disease treatment.

Also described herein methods of treating mitochondrial dysfunction in and around the eye by using an MTT-prodrug comprising a bioactive MTT that is covalently linked to an inactive conjugation moiety that facilitates noncovalent interactions between the conjugation moiety of the prodrug and a complexation agent within a dispersal medium and serves to limit the amount of free MTT-prodrug within the dispersal medium.

In general, a method of treating mitochondrial dysfunction in or around the eye may include administering any of the therapeutic compositions described herein.

Also described are methods of treating or preserving neurosensory retina structure including ellipsoid zone, treating RPE dysmorphology, RPE-associated extracellular matrix dysregulation, abnormal RPE metabolism, sub-RPE deposit, and/or drusen deposits, by administering any of the therapeutic compositions described herein to specifically enable intravitreal or periocular injections of formulations of Mito XR that produce sufficiently high sustained retina and RPE tissue levels of active drug to modify these pathologic features of disease.

Also described are methods of improving vision or preventing vision loss in patients with retinal and ocular diseases, by administering any of the therapeutic compositions described herein to specifically enable intravitreal or periocular injections of formulations of Mito XR that produce sufficiently high sustained ocular tissue levels of active drug to improve function of relevant ocular tissues.

Also described are methods of preventing onset or progression of atrophic retinal disease, e.g., geographic atrophy, by administering any of the therapeutic compositions described herein to specifically enable intravitreal or periocular injections of formulations of Mito XR that produce sufficiently high sustained retina and RPE tissue levels of active drug to restore cellular health, limit cell death, and prevent progressive loss of vital tissue.

In any of these methods the active MTT may be released via cleavage of prodrug by esterases present within the vitreous or other tissues of the eye. The active mitochondria targeted peptide may be released via hydrolysis or other reaction that results in release of the bioactive mitochondrial-targeted peptide drug. The released bioactive MTT drug may be H-d-Arg-DMT-Lys-Phe-OH, or any MTT disclosed in the list in Table 1.

Administration may comprise local ocular administration via injection of an implant of Mito XR.

Mito XR may be administered into the eye using intravitreal (IVT), periocular, sub-Tenon's, subconjunctival, suprachoroidal, or intracameral routes. The administration may comprise injecting a formulation of Mito XR as a modality of bolus into the vitreous of the eye (FIG. 33A, FIG. 34)).

Administration may comprise injecting a formulation of prodrug within the Mito XR implant (multiphasic colloidal suspension) as a modality of a sustained release drug formulation device. The extended release drug delivery system may comprise delivering a bioerodible or non-bioerodible implant into a vitreous of the eye (FIG. 33, FIG. 34).

Any of these methods may include treatment intervals of 1-12 months for administering Mito XR into the subject's eye. The method may be a method of treating retinal and optic nerve diseases, including dry age-related macular degeneration (AMD), wet AMD, diabetic retinopathy (DR), retinal vein occlusion (RVO), retinitis pigmentosa (RP), glaucoma, optic nerve disease, or for neuroprotection of the retina and/or optic nerve.

The method may be used in conjunction with other treatment modalities including inhibition of vascular endothelial growth factor, complement inhibition, or administration of anti-inflammatory drugs such as corticosteroids.

A method of treatment of mitochondrial dysfunction in a subject's eye may include delivering a MTT-prodrug incorporated into formulations of Mito XR into the subject's eye at a treatment start; and cleavage of the covalent bond of the prodrug to release the active MTT into the eye during a first phase at a burst phase release rate; subsequently during a second phase at a steady-state release rate, wherein the burst phase rate is greater than the steady state dose rate, further wherein the first phase extends from the treatment start for about 2-6 weeks and the subsequent phases (second phase, and in some instances second and third phases) extend from an end of the first phase for one or more months.

A method of treatment of RPE dysmorphology or sub-RPE deposits in a subject's eye, may include delivering a MTT-prodrug incorporated into formulations of Mito XR into the subject's eye at a treatment start; and cleavage of the covalent bond of the prodrug to release the active MTT into the eye during a first phase at a burst phase release rate; subsequently during a second phase at a steady-state release rate, wherein the burst phase rate is greater than the steady state dose rate, further wherein the first phase extends from the treatment start for about 2-6 weeks and the subsequent phases (second phase, and in some instances second and third phases) extend from an end of the first phase for one or more months.

A method of treatment of vision loss in a subject may include delivering a MTT-prodrug incorporated into formulations of Mito XR into the subject's eye at a treatment start; and cleavage of the covalent bond of the prodrug to release the active MTT into the eye during a first phase at a burst phase release rate; subsequently during a second phase at a steady-state release rate, wherein the burst phase rate is greater than the steady state dose rate, further wherein the first phase extends from the treatment start for about 2-6 weeks and the subsequent phases (second phase, and in some instances second and third phases) extend from an end of the first phase for one or more months.

A method of treatment of vision loss in a subject may include delivering a MTT-prodrug incorporated into formulations of Mito XR into the subject's eye at a treatment start; and cleavage of the covalent bond of the prodrug to release the active MTT into the eye during a first phase at a burst phase release rate; subsequently during a second phase at a steady-state release rate, wherein the burst phase rate is greater than the steady state dose rate, further wherein the first phase extends from the treatment start for about 2-6 weeks and the subsequent phases (second phase, and in some instances second and third phases) extend from an end of the first phase for one or more months.

A method of preventing onset or progression of atrophic retinal disease in a subject may include delivering a MTT-prodrug incorporated into formulations of Mito XR into the subject's eye at a treatment start; and cleavage of the covalent bond of the prodrug to release the active MTT into the eye during a first phase at a burst phase release rate; subsequently during a second phase at a steady-state release rate, wherein the burst phase rate is greater than the steady state dose rate, further wherein the first phase extends from the treatment start for about 2-6 weeks and the subsequent phases (second phase, and in some instances second and third phases) extend from an end of the first phase for one or more months

All of the methods and apparatuses described herein, in any combination, are herein contemplated and can be used to achieve the benefits as described herein.

Described herein are methods of manufacturing for Mito XR, wherein a selected MTT-prodrug is admixed with a complexation agent particulate to form MTT-prodrug-complex particulate. One or more MTT-prodrug-complex particulate(s) are then added and incorporated to a selected dispersal medium to form the stable multiphasic colloidal suspension. The resultant formulation of MTT-prodrug, complexation agents, and dispersal medium forms the implant of Mito XR (FIG. 35).

The property of Kd is a measure of avidity of an MTT-prodrug for a given complexation agent and is defined as the unbound-bound fraction of MTT-prodrug for an MTT-prodrug-complex particulate in a given dispersal medium. Specific Kd value can be measured by specified release assay, as described herein.

The regulation of release of MTT-prodrug from the implant is determined by the unbound fraction within the dispersal medium, which is in turn determined by the Kd, defined as the ratio of unbound to bound MTT-prodrug for a given complexation agent within a specific dispersal medium. Knowledge of the Kd for a particular MTT-prodrug-complex particulate allows the choice of specific combinations of prodrug-complexation agent to achieve a prespecified release kinetics profile. The inclusion of more than one complexation agent in the multiphasic colloidal suspension can be used to regulate the unbound fraction of drug within the dispersal medium over time and thus the release kinetics of the system (see FIGS. 25B, 35A-35E, FIG. 36).

For example, in some formulations of Mito XR, there may be a first phase and a second phase of release, wherein there is increased release of the mitochondrial targeted tetrapeptide during the first phase, and a subsequent lower release of mitochondrial targeted tetrapeptide during the second phase (see FIG. 36). This formulation may be achieved by the combination of two different MTT-prodrug-complex particulates, wherein one complex particulate has high Kd, reflecting low affinity of MTT-prodrug for first complexation agent) and the second complex particulate has low Kd, reflecting high affinity of MTT-prodrug for second complexation agent. In this setting, the first phase of release may be a “burst” faster rate of MTT-prodrug release from the higher Kd (low affinity) particulate, and the second phase of release is a slower, steady-state of MTT-prodrug release from the lower Kd (higher affinity) particulate. In this manner, different MTT-prodrug-complex particulates can be specifically selected and combined, in desired ratio and proportion, to achieve a prespecified kinetic profile of MTT-prodrug release from Mito XR formulation.

In such examples, the combined effect for a combination of two or more MTT-prodrug-complex particulates incorporated into selected dispersal medium is release of the MTT in two or more phases based on the integral of release rates from the individual drug-complexation agent particulate components that are incorporated and dispersed within the Mito XR implant (FIG. 35).

The actual release kinetics of achieved by Mito XR in in vivo vitreous concentrations may meet or exceed EC₅₀ for an extended-release duration of 1 month or more. The EC₅₀ reflects the concentration of the MTT-prodrug compound that achieves 50% of the maximal response for reduction in mitochondrial dysfunction measured both for reversal of pre-existing mitochondrial dysfunction and for prevention of new onset mitochondrial dysfunction, by specific readouts of mitochondrial dysfunction.

In formulations of Mito XR with two-phase release kinetics, the concentration of MTT-prodrug in the vitreous may exceed the reversal EC₅₀ during the initial burst phase and subsequently exceed the prevention EC₅₀ for the second (steady-state) phase, and release kinetics, selection of specific MTT-prodrug-complex particulates, specific ratio and proportion of different particulate combinations, and total payload of MTT-prodrug in the Mito XR formulation may be selected to achieve this designed release kinetics for desired duration of drug release.

In formulations of Mito XR with single-phase release kinetics, the concentration of MTT-prodrug in the vitreous may exceed the EC₅₀ for reversal of mitochondrial dysfunction.

In formulations of Mito XR with three-phase release kinetics, the concentration of MTT-prodrug in the vitreous may exceed the EC₅₀ for reversal of mitochondrial dysfunction during the first phase, may exceed the EC₅₀ for prevention of mitochondrial dysfunction for steady-state release during the second phase, and may exceed EC₅₀ for reversal of mitochondrial dysfunction during the third, late-burst phase.

The multiphasic colloidal suspension may be formulated as one of several modalities of the complexation-based extended-release drug delivery system that may be injected into the vitreous (FIGS. 33A-33C and 34), including a flowable bolus implant (FIG. 33A), a solid mold of a specific size and shape, or a semi-solid that fills a bioerodible or non-bioerodible sleeve or outer covering to form a tube implant (FIG. 33B). In some examples, the tube may itself be formed of the extended release drug delivery system. In other examples, the tube may be a comprised of a bio-erodible polymer that is compatible with ocular tissues (e.g., poly(lactic-co-glycolic acid) PLGA). In some examples, the tube may have one or both ends open for release of the MTT-prodrug. The tube may be injected via needle or cannula (FIG. 33B) into the vitreous, as shown in FIG. 34 (right) or into periocular tissues. In some examples, the extended release drug delivery system incorporating MTT-prodrug may be molded into shapes (FIG. 33C).

For example, described herein are prodrug compounds comprising a mitochondrial targeted tetrapeptide (MTT) (e.g., any of the MTTs from SEQ ID NOs. 1-635) containing alternating cationic and aromatic amino acid residues, that is linked to a conjugation moiety by a cleavable covalent bond.

In some examples the prodrug compound has the formula: R′—R where R′ is a mitochondrial targeted tetrapeptide (MTT) containing alternating cationic and aromatic amino acid residues in which the C-terminal amino acid is covalently linked to R by a cleavable covalent bond, where R is a conjugation moiety that may be removed by enzymatic cleavage, catalysis, hydrolysis, or other reaction to yield free mitochondria targeted tetrapeptide R′ and conjugation moiety R, where R is selected from: a C4-C30 lipid moiety (fatty acid or fatty alcohol), an C4-C30 straight-chain or branched aliphatic moiety, a 2-mer to 30-mer peptide moiety, a pegylated moiety, or a carbohydrate moiety.

In some examples the prodrug compound has the formula: R′(—O)—R where R′ is a mitochondrial targeted tetrapeptide (MTT) containing alternating cationic and aromatic amino acid residues in which the C-terminal amino acid hydroxyl group is linked via an ester bond to R, where R or —O—R is a conjugation moiety selected from: a C4-C30 lipid moiety (fatty acid or fatty alcohol), an C4-C30 straight-chain or branched aliphatic moiety, a 2-mer to 30-mer peptide moiety, a pegylated moiety, or a carbohydrate moiety.

In some examples the prodrug compound has the formula of: H-d-Arg-DMT-Lys-Phe(-O)—R (designated as EY005-R):

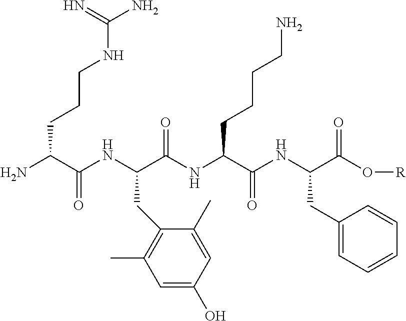

where the fourth amino acid is linked via ester bond to R, and R or —O—R is a conjugation moiety selected from: a C4-C30 lipid moiety, (fatty acid or fatty alcohol), an C4-C30 straight-chain or branched aliphatic moiety, a 2-mer to 30-mer peptide moiety, or a pegylated moiety, or a carbohydrate moiety.

In some examples the prodrug compound has one of the following formulas: H-d-Arg-DMT-Lys-Phe(-O)-octadecyl; H-d-Arg-DMT-Lys-Phe(-O)-Arg₍ₙ₎, where n is between 1 and 30; H-d-Arg-DMT-Lys-Phe(-O)-Glu₍ₙ₎, where n is between 1 and 30. H-d-Arg-DMT-Lys-Phe(-O)-octadecyl is also referred to equivalently as H-d-Arg-DMT-Lys-Phe(-O)-stearyl.

Also described herein are compositions of a multiphasic colloidal suspension comprising a mitochondrial targeted tetrapeptide (MTT)-prodrug and one or more complexation agents, admixed in a dispersal medium. The MTT is a mitochondria targeted peptide having sequence from one of SEQ ID NO 1-635. The complexation agent may be a chemical substance formulated as an irregular shaped particulate, capable of forming MTT-prodrug-complex particulates, selected from one of six classes: fatty acid, organic compounds that can form keto-enol tautomers, charged phospholipid, charged protein, ribonucleic acid, and polysaccharide. For example, the complexation agent may be a fatty acid, which is a carboxylic acid with an aliphatic chain with chemical formula of CH3(CH2)ₙCOOH where n is equal to between 4 and 30, which may be either saturated or unsaturated and may be in the form of a salt or ester, and includes the following: magnesium palmitate, magnesium stearate, calcium palmitate, calcium stearate. The complexation agent may be one or more of: organic compounds that can form keto-enol tautomers, molecules capable of undergoing chemical equilibrium between a keto form (a ketone or an aldehyde) and an enol form (an alcohol) and includes the following: phenol compound, tocopherol compound, quinone compound, ribonucleic acid compound. The complexation agent may be one or more of: a charged phospholipid and includes the following: anionic phospholipid, lecithin, phosphatidylcholine, phosphatidylethanolamine, phosphatidylserine, sphingomyelin, synthetic phospholipids with positive charge, DLin-MC3-DMA. The complexation agent may be a charged protein that may be positive or negative and includes albumin, synthetic polypeptides, plasma proteins, alpha2-macroglobulin, fibrin, collagen. In some examples the complexation agent is a ribonucleic acid comprising a biopolymer macromolecule comprising nucleotides, comprising a 5-carbon sugar, a phosphate group, and a nitrogenous base. In some examples the complexation agent is a polysaccharide, long chain polymeric carbohydrates comprising monosaccharide units bound together by glycosidic linkages and includes: ringed polysaccharide molecule, cyclodextrin, clathrate.

The dispersal medium may be capable of forming multiphasic colloidal suspension, and may be selected from among four classes of hydrophobic oils, which are not previously known to form a multiphasic colloidal suspension when admixed with selected MTT-prodrug-complex particulates: saturated fatty acid methyl esters, unsaturated fatty acid methyl esters, saturated fatty acid ethyl esters, unsaturated fatty acid ethyl esters. The dispersal medium may comprise a saturated fatty acid methyl esters comprising one or more of: methyl acetate, methyl propionate, methyl butyrate, methyl pentanoate, methyl hexanoate, methyl heptanoate, methyl octanoate, methyl nonanoate, methyl decanoate, methyl undecanoate, methyl dodecanoate (methyl laurate), methyl tridecanoate, methyl tetradecanoate, methyl 9(Z)-tetradecenoate, methyl pentadecanoate, methyl hexadecanoate, methyl heptadecanoate, methyl octadecenoate, methyl nonadecanoate, methyl eicosanoate, methyl heneicosanoate, methyl docosanoate, and methyl tricosanoate. The dispersal medium may comprise an unsaturated fatty acid methyl esters comprising one or more of: methyl 10-undecenoate, methyl 11-dodecenoate, methyl 12-tridecenoate, methyl 9(E)-tetradecenoate, methyl 10(Z)-pentadecenoate, methyl 10(E)-pentadecenoate, methyl 14-pentadecenoate, methyl 9(Z)-hexadecenoate, methyl 9(E)-hexadecenoate, methyl 6(Z)-hexadecenoate, methyl 7(Z))-hexadecenoate, methyl 11(Z)-hexadecenoate. The dispersal medium may comprise a saturated fatty acid ethyl esters comprising one or more of: ethyl acetate, ethyl propionate, ethyl butyrate, ethyl pentanoate, ethyl hexanoate, ethyl heptanoate, ethyl octanoate, ethyl nonanoate, ethyl decanoate, ethyl undecanoate, ethyl dodecanoate (ethyl laurate), ethyl tridecanoate, ethyl tetradecanoate, ethyl 9(Z)-tetradecenoate, ethyl pentadecanoate, ethyl hexadecanoate, ethyl heptadecanoate, ethyl octadecenoate, ethyl nonadecanoate, ethyl eicosanoate, ethyl heneicosanoate, ethyl docosanoate, and ethyl tricosanoate. In some examples, the dispersal medium may comprise an unsaturated fatty acid ethyl esters comprising one or more of: ethyl 10-undecenoate, ethyl 11-dodecenoate, ethyl 12-tridecenoate, ethyl 9(E)-tetradecenoate, ethyl 10(Z)-pentadecenoate, ethyl 10(E)-pentadecenoate, ethyl 14-pentadecenoate, ethyl 9(Z)-hexadecenoate, ethyl 9(E)-hexadecenoate, ethyl 6(Z)-hexadecenoate, ethyl 7(Z))-hexadecenoate, ethyl 11(Z)-hexadecenoate.

Also described herein are methods of using a mitochondrial targeted tetrapeptide (MTT)-prodrug multiphasic colloidal suspension to treat a mitochondrial disorders of the eye, the method comprising administering the MTT-prodrug multiphasic colloidal suspension by local ocular administration, including one or more of: intravitreal (IVT), periocular, sub-Tenon's, subconjunctival, suprachoroidal, or intracameral routes of administration. The MTT-prodrug multiphasic colloidal suspension may be used to treat a mitochondrial disorders of the eye by one or more of: preventing onset or slow progression, preventing vision loss or improve vision, preventing onset or improving destructive or degenerative aspects of ocular conditions and diseases, including one or more of: dry age-related macular degeneration (AMD), wet AMD, diabetic macular edema (DME), retinal vein occlusion (RVO), and inherited retinal degeneration (IRD), retinal degeneration, traumatic injury, ischemic vasculopathy, acquired or hereditary optic neuropathy, glaucoma, endophthalmitis, retinitis, uveitis, inflammatory diseases of the retina and uveal tract, Fuch's corneal dystrophy, corneal edema, ocular surface disease, dry eye disease, chronic progressive external ophthalmoloplegia (CPEO), diseases of the conjunctiva, diseases of the periocular tissue, and diseases of the orbit.

Administering the MTT-prodrug multiphasic colloidal suspension by local ocular administration may comprise administering from an implant, wherein a combination of reversible, noncovalent complexation of a conjugation moiety of the MTT-prodrug multiphasic colloidal suspension to irregularly shaped particulate complexation agents, forming an MTT-prodrug-complex particulates, and stable dispersal of the MTT-prodrug-complex particulates within a hydrophobic dispersal medium limits free MTT-prodrug available for release from the implant into the ocular physiologic environment.

Treating the mitochondrial disorders of the eye may comprise treatment of retinal pigment epithelium (RPE) dysmorphology, RPE-associated extracellular matrix dysregulation, and/or sub-RPE deposits in human patients or animals by continuous, sustained exposure of RPE and retina tissue to therapeutic levels of the MTT-prodrug multiphasic colloidal suspension by intravitreal or periocular injection of formulations of the MTT-prodrug multiphasic colloidal suspension. In some examples, treating the mitochondrial disorders of the eye comprises improving vision or preventing vision loss in patients, by continuous, sustained exposure of retinal pigment epithelium (RPE) and retina tissue to therapeutic levels of the MTT-prodrug multiphasic colloidal suspension by intravitreal or periocular injection of formulations of the MTT-prodrug multiphasic colloidal suspension.

For example, a method of treatment of mitochondrial dysfunction in a subject's eye may include: delivering a prodrug of a mitochondrial targeted tetrapeptide combined with the extended release drug delivery system into the subject's eye at a treatment start; and cleaving, by action of an esterase in the subject's eye, the prodrug to release the mitochondrial targeted tetrapeptide into the eye during a first phase at a burst phase release rate; and cleaving, by action of the esterase, the prodrug to release the mitochondrial targeted tetrapeptide into the eye during a second phase at a steady-state release rate, wherein the burst phase release rate is greater than the steady state release rate, further wherein the first phase extends from the treatment start for about 2-6 weeks and the second phase extend from an end of the first phase for one or more months.

A method of treatment of retinal pigment epithelium (RPE) dysmorphology or sub-RPE deposits in a subject's eye by local intravitreal or periocular injections of formulations in extended release drug delivery system that produce high sustained retina and retinal pigment epithelium (RPE) tissue levels of active drug may comprise: delivering a prodrug of a mitochondrial targeted tetrapeptide combined with the extended release drug delivery system into the subject's eye at a treatment start; cleaving, by action of an esterase in the subject's eye, the prodrug to release the mitochondrial targeted tetrapeptide into the eye during a first phase at a burst phase release rate; and cleaving, by action of the esterase, the prodrug to release the mitochondrial targeted tetrapeptide into the eye during a second phase at a steady-state release rate, wherein the burst phase release rate is greater than the steady state release rate, further wherein the first phase extends from the treatment start for about 2-6 weeks and the second phase extend from an end of the first phase for one or more months.

In some examples, a method of treatment of vision loss in a subject by intravitreal or periocular injections of formulations in extended release drug delivery system that produce high sustained retina and retinal pigment epithelium (RPE) tissue levels of active drug includes: delivering a prodrug of a mitochondrial targeted tetrapeptide combined with the extended release drug delivery system into the subject's eye at a treatment start; and cleaving, by action of an esterase in the subject's eye, the prodrug to release the mitochondrial targeted tetrapeptide into the eye during a first phase at a burst phase release rate; and cleaving, by action of the esterase, the prodrug to release the mitochondrial targeted tetrapeptide into the eye during a second phase at a steady-state release rate, wherein the burst phase release rate is greater than the steady state release rate, further wherein the first phase extends from the treatment start for about 2-6 weeks followed by the second phase.

A method of preventing onset of atrophy or slowing progression of atrophy of the neurosensory retina and/or retinal pigment epithelium (RPE) in a subject by intravitreal or periocular injections of formulations of an extended release drug delivery system that produces high sustained retina and RPE tissue levels of active drug may include: delivering a prodrug of a mitochondrial targeted tetrapeptide combined with the extended release drug delivery system into the subject's eye at a treatment start; and cleaving, by action of an esterase in the subject's eye, the prodrug to release the mitochondrial targeted tetrapeptide into the eye during a first phase at a burst phase release rate; and cleaving, by action of the esterase, the prodrug to release the mitochondrial targeted tetrapeptide into the eye during a second phase at a steady-state release rate, wherein the burst phase release rate is greater than the steady state release rate, further wherein the first phase extends from the treatment start for about 2-6 weeks and the second phase extends thereafter. In any of these methods the prodrug may be any of the prodrugs (e.g., the MTT-prodrugs) described herein.

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1A illustrates the normal action of the specialized mitochondrial lipid cardiolipin (201) in organizing the complexes of the electron transport chain (205) in the mitochondria, specifically at the tip of the cristae (203) at the inner mitochondrial membrane.

FIG. 1B shows a disruption of complexes of the electron transport chain that occurs when cardiolipin is peroxidized, which results in mitochondrial dysfunction: diminished ATP production, increased production of superoxide and reactive oxygen species, calcium dysregulation, etc.

FIG. 1C shows re-approximation of complexes of the electron transport chain in the mitochondria and reversal of mitochondrial dysfunction (211) following restoration of cardiolipin function by use of a mitochondrial-targeted tetrapeptide, which binds and restores function of peroxidized cardiolipin (209), as described herein.

FIG. 2A illustrates the unexpected and nonobvious effects of treatment with high dose (3 mg/kg) subcutaneous treatment of the APOE4+high fat diet (HFD) mouse model of dry AMD with one example of mitochondrial targeted tetrapeptide (elamipretide), with regression of subRPE deposits and restoration of normal outer retinal and RPE morphology, as shown by electron microscopy of the Sub-RPE and outer retina region of the eye.

FIG. 2B is a graph quantifying the unexpected and nonobvious effects of treatment with high dose (3 mg/kg) subcutaneous treatment of the APOE4+high fat diet (HFD) mouse model of dry AMD with one example of mitochondrial targeted tetrapeptide (elamipretide), with reversal of visual dysfunction and improved vision as reflected by increased B-wave amplitudes on ERG testing following elamipretide treatment in mice.

FIG. 3 illustrates summary results of the ReCLAIM study, a phase 1/2 clinical trial of systemically administered (40 mg subcutaneous daily) elamipretide in two cohorts of patients with dry AMD: 1) noncentral geographic atrophy (GA) and 2) high-risk drusen. While there was a general mean trend toward improvement in multiple metrics of visual function, especially in low-luminance visual function, only 50% of patients demonstrated a response to therapy, in a setting when patients received the maximally tolerated dose of 40 mg once daily (0.4-0.9 mg/kg), nearly one-tenth the dosage administered in the APOE4+high fat diet (HFD) mouse model of dry AMD, where strongly positive effects on visual function and disease morphology were observed.

FIG. 4A shows the structure of one example of a mitochondrial targeted tetrapeptide, H-d-Arg-DMT-Lys-Phe (“EY005”) that may be formed as a prodrug by covalent bond to the carboxylic acid moiety, as described herein.

FIG. 4B illustrates one example of a formula of a prodrug for a mitochondrial targeted tetrapeptide (based on EY005, shown in FIG. 4A), where “R” is a conjugation moiety, as described herein.

FIG. 5 shows one example of a cell culture protocol using retinal pigment epithelium (RPE) cells for assaying the effect of a mitochondrial targeted tetrapeptide (MTT), or prodrug of an MTT, as described herein, in which mitochondrial dysfunction is induced by two exposures to nonlethal doses of hydroquinone (HQ) at day −3 and day 0. Cells are then treated with drug (or control phosphate-buffered saline (PBS) solution) and assayed for effects on mitochondrial dysfunction and other cellular changes.

FIG. 6 illustrates the effects of one example of a mitochondrial targeted tetrapeptide (EY005) on the HQ cell culture model of dry AMD, with reversal of induced mitochondrial dysfunction in cultured RPE cells, as reflected by reduced flavoprotein autofluorescence, a marker of electron transport chain complex II dysfunction.

FIG. 7 illustrates unexpected and nonobvious effects of treatment with high dose (≥5 μM) mitochondrial targeted tetrapeptide (EY005), with reversal of dysregulated extracellular matrix in cultured RPE cells as reflected by reduced vimentin expression and reversal of actin cytoskeleton disorganization as reflected by phalloidin staining.

FIGS. 8A-8B are graphs demonstrating reversal of mitochondrial dysfunction as reflected by reduction of flavoprotein autofluorescence following treatment with mitochondrial targeted peptide, with EY005 (FIG. 8B) demonstrating equivalent efficacy and potency as compared to elamipretide (FIG. 8A).

FIGS. 9A-9B are graphs showing the reversal of dysregulated extracellular matrix (vimentin) by mitochondrial targeted tetrapeptide in cultured RPE cells, with dose-response studies demonstrating equivalent efficacy and potency of EY005 (FIG. 9B) as compared to elamipretide (FIG. 9A).

FIGS. 10A-10B are graphs showing the reversal of actin cytoskeleton disorganization by mitochondrial targeted tetrapeptide in cultured RPE cells, with dose-response studies demonstrating equivalent efficacy and potency of EY005 (FIG. 10B) as compared to elamipretide (FIG. 10A).

FIG. 11A-11B shows two markers of mitochondrial dysfunction, flavoprotein autofluorescence (FP-AF) (FIG. 11A) and superoxide (FIG. 11B), evaluated by microscopy of mouse retinal tissues. Control, untreated mice show undetectable FP-AF and superoxide. Following experimental retinal vein occlusion (RVO), FP-AF and superoxide are greatly increased. Systemic treatment with EY005 (3 mg/kg, BID) administered subcutaneously significantly attenuates both markers of mitochondrial dysfunction following RVO.

FIG. 11C shows two markers of synaptic integrity, synaptic vesicle protein 2 (SV2) and synaptotagmin (ZNP-1) evaluated by microscopy in both the inner and outer plexiform layers of mouse retinal tissues. Control, untreated mice show normal synaptic markers. Following experimental retinal vein occlusion (RVO), synaptic markers in both the inner and outer plexiform layers are greatly reduced. Systemic treatment with EY005 (3 mg/kg, BID) administered subcutaneously significantly prevents (pretreatment) and reverses (posttreatment) loss of synaptic markers following RVO.

FIGS. 11D-11G quantify the data from FIG. 11C from at least 5 independent experiments and quantification of synaptic markers in the inner and outer plexiform layers are displayed in graphical form.

FIGS. 11H-111 show visual function as quantified by electroretinogram (ERG) in mice treated with control, RVO and RVO with EY005 pretreatment or EY005 post-treatment. Representative ERGs are shown in FIG. 11H. B-wave amplitudes were quantified from at least 5 eyes for each condition. RVO resulted in near total loss of B-wave. However, both pretreatment and post-treatment significantly prevented or reversed loss of B-wave amplitude, respectively (FIG. 11I).

FIG. 12 illustrates one example of a relevant animal model, specifically a rabbit-based protocol used to characterize the prodrugs of mitochondrial targeted tetrapeptides described herein. In this model, HQ (0.05 mL, 250 mM HQ) is injected into the vitreous cavity of rabbit eye on day 0 and day 1, and drug of interest (mitochondrial targeted peptide or prodrug thereof) or control (PBS) solution is injected into the same vitreous cavity of the rabbit eye at day 2. At day 4, the rabbit is euthanized, and eyes recovered for histologic analysis of retina and RPE flatmounts for various metrics, including cellular oxidation (DCFDA), as a surrogate of mitochondrial dysfunction, as well as for other cellular markers.

FIG. 13 shows one example of the restorative effect of a mitochondrial targeted tetrapeptide (EY005, 15 μM) on induced mitochondrial dysfunction in RPE cells of rabbit eyes, with reduced oxidant species, within 36 hours of treatment, in the rabbit model detailed in FIG. 12.

FIG. 14 illustrates unexpected and nonobvious effects of treatment with high dose intravitreal mitochondrial targeted tetrapeptide EY005 (15 μM) in the rabbit model detailed in FIG. 12, with reversal of dysregulated extracellular matrix in RPE cells as reflected by reduced vimentin expression and reversal of RPE cell actin cytoskeleton disorganization as reflected by phalloidin staining.

FIGS. 15A-15D shows representative histology specimens which demonstrate the superior efficacy of intravitreally administered EY005 (15 μM) compared to systemically administered EY005 (0.9 mg/kg subcutaneous (SQ) once daily, dosage modeled after dose used in human clinical trial). The rabbit hydroquinone (HQ) model of dry AMD (FIG. 12) was induced and flatmount of retinal pigment epithelium (RPE) were isolated. Compared to control RPE (FIG. 15A), HQ-exposed rabbits showed severe disorganization of the cortical actin cytoskeleton as well as punctate actin intracellular deposits (FIG. 15B). Treatment with intravitreal EY005 (15 μM) resulted in near complete reversal of RPE cytoskeletal dysmorphology (FIG. 15C). By contrast, subcutaneous administration of EY005 (0.9 mg/kg, once daily) resulted in only partial reversal of RPE cytoskeletal dysmorphology (FIG. 15D).

FIG. 15E shows graph depicting RPE dysmorphology severity scores from rabbits treated as in FIGS. 15A-15D. Scores were determined by masked expert graders in at least 100 RPE cells from at least 3 eyes per condition.

FIG. 16A is an example of a prodrug of the EY005 mitochondrial targeted tetrapeptide including a stearyl alcohol or octadecyl moiety linked by ester bond to the mitochondrial targeted tetrapeptide.

FIG. 16B is an example of a prodrug of the EY005 mitochondrial targeted tetrapeptide including a peptide motif (e.g., an anionic tri-Glu peptide) and linker moiety linked via ester bond to EY005.

FIG. 16C is an example of a prodrug of the EY005 mitochondrial targeted tetrapeptide including a peptide motif (e.g., a cationic tri-Arg peptide) and linker moiety linked via ester bond to EY005.

FIG. 16D is an example of a prodrug of the EY005 mitochondrial targeted tetrapeptide including a polyethylene glycol (PEG) linked by ester bond to EY005.

FIGS. 17A-17C demonstrates cleavage of ester based EY005-stearyl prodrug by carboxyesterase and by spontaneous hydrolysis. FIG. 17A shows baseline HPLC analysis of EY005-stearyl prodrug (top tracing) and EY005 MTT (bottom tracing). EY005-stearyl was incubated at 37° C. in vitro with carboxyesterase (0.1 μg/mL), to simulate the ocular physiologic environment and the type of esterase that is readily abundant within the vitreous. Incubation of EY005-stearyl with carboxyesterase produced rapid cleavage of the prodrug ester bond, releasing EY005, as evident by disappearance of the EY005-stearyl prodrug peak and appearance of the EY005 peak on high performance liquid chromatography (FIG. 17B). Upon addition of EY005-stearyl prodrug to phosphate-buffered saline solution at 37° C. without esterase, the ester bond of the EY005-stearyl prodrug cleaves more slowly by hydrolysis (FIG. 17C). After 6 hours, partial cleavage of EY005-stearyl prodrug to EY005 MTT is noted.

FIG. 18 illustrates theoretical data from an in vitro esterase assay showing rapid ester cleavage of two different EY005 prodrugs upon exposure to the esterase.

FIG. 19A shows an in vitro culture model of dry AMD in which RPE cells possessing endogenous esterases were exposed to hydroquinone (HQ) to induce mitochondrial dysfunction. Mitochondrial dysfunction is manifest as increased flavoprotein autofluorescence (upper panels) and as dysmorphology of the actin cytoskeleton (lower panels). EY005-stearyl (5 μM) effectively reversed HQ-induced mitochondrial dysfunction in RPE cells (as depicted by reduced cellular flavoprotein-autofluorescence and normalized actin cytoskeleton dysfunction), with efficacy equivalent to treatment with EY005 native peptide (5 μM). EY005-stearyl was also preincubated with carboxyesterase (0.1 μg/mL) in separate media. Recovered media containing cleaved EY005 (5 μM) was added to this RPE cellular model of mitochondrial dysfunction, and this was similarly effective and equipotent to EY005 native peptide for the reversal of RPE mitochondrial dysfunction.

FIG. 19B shows quantification of flavoprotein autofluorescence (FP-AF) from at least 3 replicates of each condition represented in FIG. 19A. Both EY005-stearyl and esterase-cleaved EY005-stearyl show potency equal to the native EY005 peptide.

FIG. 19C shows quantification of actin cytoskeletal dysmorphology from at least 3 replicates of each condition represented in FIG. 19A. Both EY005-stearyl and esterase-cleaved EY005-stearyl show potency equal to the native EY005 peptide.

FIGS. 20A-20D shows magnesium stearate imaged under various conditions to assess complexation with FITC-labeled EY005-stearyl prodrug. FIG. 20A shows Magnesium stearate alone shows low intrinsic fluorescence. FIG. 20B shows Magnesium stearate incubated with FITC-labeled EY005 native peptide. FITC-labeled EY005 native peptide alone showed minimal complexation with magnesium stearate as reflected by minimal fluorescent labeling of particulates. FIG. 20C shows Magnesium stearate incubated with FITC-labeled EY005 prodrug. Complexation of prodrug with magnesium stearate was evident as moderate fluorescence of imaged magnesium stearate particulates.

FIG. 20D shows treating FITC-labeled EY005-stearyl prodrug complexed with magnesium from sample C with carboxyesterase (0.1 μg/mL) reduced levels of fluorescence demonstrating cleavage of EY005-stearyl prodrug with release of labeled EY005 peptide.

FIGS. 21A-21D show albumin imaged under various conditions to assess complexation with FITC-labeled EY005-stearyl prodrug. FIG. 21A shows Albumin alone shows low intrinsic fluorescence.

FIG. 21B shows Albumin incubated with FITC-labeled EY005 native peptide. FITC-labeled EY005 native peptide alone showed minimal complexation with albumin as reflected by negative staining of albumin crystals surrounded by generalized fluorescence from dissolved FITC-labeled EY005 native peptide. FIG. 21C shows Albumin incubated with FITC-labeled EY005 prodrug. Complexation of EY005-stearyl prodrug with albumin was evident as bright fluorescence of imaged albumin crystals. FIG. 21D shows treating FITC-labeled EY005-stearyl prodrug complexed with albumin from sample C with carboxyesterase (0.1 μg/mL) reduced levels of fluorescence demonstrating cleavage of EY005-stearyl prodrug with release of labeled EY005 peptide.

FIGS. 22A-22D show cyclodextrin gamma imaged under various conditions to assess complexation with FITC-labeled EY005-stearyl prodrug. FIG. 22A shows Cyclodextrin alone shows low intrinsic fluorescence. FIG. 22B shows Cyclodextrin incubated with FITC-labeled EY005 native peptide. FITC-labeled EY005 native peptide alone showed minimal complexation with cyclodextrin as reflected by minimal increase in fluorescence above that of cyclodextrin alone. FIG. 22C shows Cyclodextrin incubated with FITC-labeled EY005 prodrug. Complexation of EY005-stearyl prodrug with cyclodextrin was evident as moderated fluorescence of imaged cyclodextrin particulates. FIG. 22D shows treating FITC-labeled EY005-stearyl prodrug complexed with cyclodextrin from sample C with carboxyesterase (0.1 μg/mL) reduced levels of fluorescence demonstrating cleavage of EY005-stearyl prodrug with release of labeled EY005 peptide.

FIGS. 23A-23D show lecithin imaged under various conditions to assess complexation with FITC-labeled EY005-stearyl prodrug. FIG. 23A shows Lecithin alone shows low intrinsic fluorescence.

FIG. 23B shows Lecithin incubated with FITC-labeled EY005 native peptide. FITC-labeled EY005 native peptide showed minimal complexation with lecithin as reflected by minimal increase in fluorescence above that of lecithin alone. FIG. 23C shows Lecithin incubated with FITC-labeled EY005 prodrug. Complexation of EY005-stearyl prodrug with lecithin was evident as bright fluorescence of all lecithin samples. FIG. 23D shows treating FITC-labeled EY005-stearyl prodrug complexed with lecithin from sample C with carboxyesterase (0.1 μg/mL) reduced levels of fluorescence demonstrating cleavage of EY005-stearyl prodrug with release of labeled EY005 peptide.

FIGS. 24A-24D shows silica microbeads imaged under various conditions to assess complexation with FITC-labeled EY005-stearyl prodrug. FIG. 24A shows Silica microbeads alone show minimal intrinsic fluorescence. FIG. 24B shows Silica microbeads incubated with FITC-labeled EY005 native peptide. FITC-labeled EY005 native peptide alone interacted with silica microbeads creating fluorescent round figures. FIG. 24C shows Silica microbeads incubated with FITC-labeled EY005-stearyl prodrug. There was no evidence of complexation of prodrug with silica microbeads. FIG. 24D shows treating FITC-labeled EY005-stearyl prodrug complexed with silica microbeads from sample C with carboxyesterase (0.1 μg/mL) did not alter extremely low levels of fluorescence.

FIG. 25A shows data from an accelerated in vitro release assay in which EY005 MTT was formulated with various complexation agents using methyl laurate as a dispersal medium. In all cases, all EY005 was rapidly released, in some cases within hours, and in all cases by day 3.

FIG. 25B shows data from an accelerated in vitro release assay in which EY005-stearyl prodrug was formulated with various complexation agents using methyl laurate as a dispersal medium. Formulations with no complexation agent show rapid release of EY005 into the media. A formulation with silica microbeads, which does not complex with EY005-stearyl prodrug also shows rapid release of EY005 into the media. By contrast, formulations with other complexation agents demonstrate sustained release of EY005 at varying rates. Of particular note, when magnesium stearate and albumin are both used as complexation agents in the same formulation, EY005 is released at a rate intermediate between that of formulations using either complexation agent alone.

FIG. 26 schematically illustrates one example of a mitochondrial-targeted tetrapeptide EY005 (103), which when linked to one of several classes of conjugation moieties (105), comprises a mitochondrial-targeted peptide prodrug (101). This mitochondrial-targeted peptide prodrug can be injected into the vitreous of eye (109), as part of an intravitreal (IVT) extended release drug delivery system (107), as described herein.

FIG. 27 shows in vitro pharmacokinetics of a pilot formulation of Mito XR (triangles). Mito XR achieved zero-order (i.e., linear) kinetics of EY005 bioactive tetrapeptide release, achieving the desired durability of drug release of three months, with free bioactive MTT within the dispersal medium released from the implant into the ocular physiologic environment. By contrast, identically formulated EY005 native peptide (circles) had extremely rapid release and did not provide desired durability of drug release.

FIGS. 28A-28C depicts an in vitro culture model of dry AMD in which RPE cells possessing endogenous esterases were exposed to hydroquinone (HQ) to induce mitochondrial dysfunction. Mitochondrial dysfunction is manifest as dysmorphology of the actin cytoskeleton. In these efficacy studies, a bolus implant of Mito XR (EY005-stearyl formulated in multiphasic colloidal suspension) was added to RPE cell culture model of dry AMD with endogenous esterases present. Treatment with Mito XR implant resulted in reversal of mitochondrial dysfunction and concurrent restoration of actin cytoskeletal morphology.

FIG. 28D shows a graphical representation of data from FIGS. 28A-28C. Cultured RPE cells were graded for severity of actin cytoskeletal dysmorphology in control, HQ-exposed cells and HQ-exposed cells treated with Mito XR. Results from at least 3 replicates were quantified. Cultures treated with Mito XR demonstrate an 80% reduction in severity of RPE cell actin cytoskeletal dysmorphology compared to control, HQ-exposed cells.

FIG. 29 shows superior in vivo pharmacokinetics of EY005-stearyl prodrug formulated as Mito XR. Rabbits were injected with intravitreal Mito XR implant containing formulated EY005-stearyl prodrug (EY005-stearyl release from IVT MitoXR) or with identical bolus formulation containing EY005 native peptide (EY005 peptide release from formulated bolus). EY005-stearyl prodrug formulated as Mito XR showed retinal tissue concentrations exceeding the EC₅₀ for reversal of mitochondrial dysfunction. These therapeutic drug levels were sustained through the 7-week timepoint, at which recovered Mito XR implants still contained 50% payload, indicating that this formulation will achieve desired 90-day durability. By contrast, native EY005 peptide was rapidly released with tissue concentrations nearly undetectable by week 3.5. Recovered implants contained no EY005 native peptide suggesting rapid dumping of drug in vivo.

FIGS. 30A-30B show preliminary clinical tolerability and retinal histology in rabbit eyes after Mito XR injection. Rabbits treated with Mito XR implants (arrowheads, FIG. 30A) showed no evidence of inflammation or other signs of toxicity on clinical examination. Postmortem histological assessment (FIG. 30B) showed preserved normal retinal morphology without evidence of inflammation or degeneration.

FIG. 31 illustrates theoretical in vivo release pharmacokinetic data comparing release kinetics of unmodified EY005 from the novel extended-release drug delivery system to that of two EY005 prodrugs, each of which is also formulated in the novel extended-release drug delivery system and injected into the vitreous cavity of rabbit eyes. Whereas unmodified EY005 is nearly completely released by approximately 30 days, the EY005 prodrugs incorporated with complexation agents demonstrate slowed release with approximately 40-50% release by 100 days in vivo.

FIG. 32 illustrates theoretical in vivo release pharmacokinetic data comparing release kinetics of unmodified EY005 from the novel extended-release drug delivery system to that of two EY005 prodrugs, each of which is also formulated in the novel extended-release drug delivery system and injected into the vitreous cavity of rabbit eyes. Whereas unmodified EY005 is nearly completely released by approximately 30 days, the EY005 prodrugs incorporated with complexation agents demonstrate slowed release with 100% complete release occurring by approximately 190-200 days in vivo.

FIGS. 33A-33C illustrate examples of delivery forms or modalities, for delivering implants of any of the multiphasic colloidal suspension extended-release drug delivery systems, which may be comprising one or more complexation agents noncovalently complexed with a prodrug of a mitochondrial-targeted tetrapeptide. FIG. 33A shows an example of a bolus injection in which the extended-release drug delivery system material is formulated as an injectable liquid bolus. FIG. 33B is an example in which the MTT-prodrug multiphasic colloidal suspension is formulated as a tube implant with a biodegradable outer sleeve/tubing filled with multiphasic colloidal suspension containing prodrug and complexation agents. FIG. 33C is an example in which the extended-release drug delivery system material is molded into a particular shape with solid state for implantation.

FIG. 34 illustrates two methods of injecting formulations of the multiphasic colloidal suspension into the eyes, either as a bolus injection or a tube implant.

FIGS. 35A-35E illustrates one approach for custom design of a formulation for specific pharmacokinetics release profile by mathematical formula using the complexation-based extended-release drug delivery system, including in this specific example, one method of configuring a two-phase release profile (FIG. 35A) of an extended-release drug delivery system for release of a mitochondrial targeted tetrapeptide from a prodrug form as described herein.

FIG. 36 illustrates data from a release assay of two representative formulations of EY005 prodrugs in the extended-release drug delivery system, with different durations of release. One example (formulation 2) demonstrates two-phase release kinetics, showing a first early burst phase and a second later steady-state phase.

### DETAILED DESCRIPTION

Described herein are compositions of matter, formulations, methods of manufacture, and methods of use for an extended release drug delivery system (XRDDS) comprising: mitochondria-targeted tetrapeptide (MTT)-prodrug, noncovalently interacting with one or more complexation agent particulates to form MTT-prodrug-complex particulates, admixed within a hydrophobic dispersal medium, that collectively forms a stable multiphasic colloidal suspension.

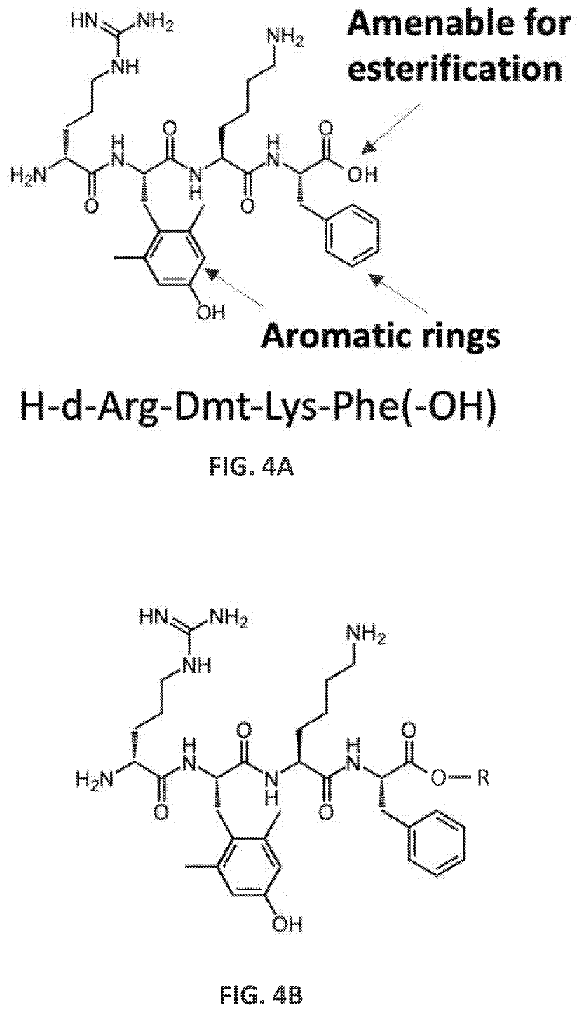

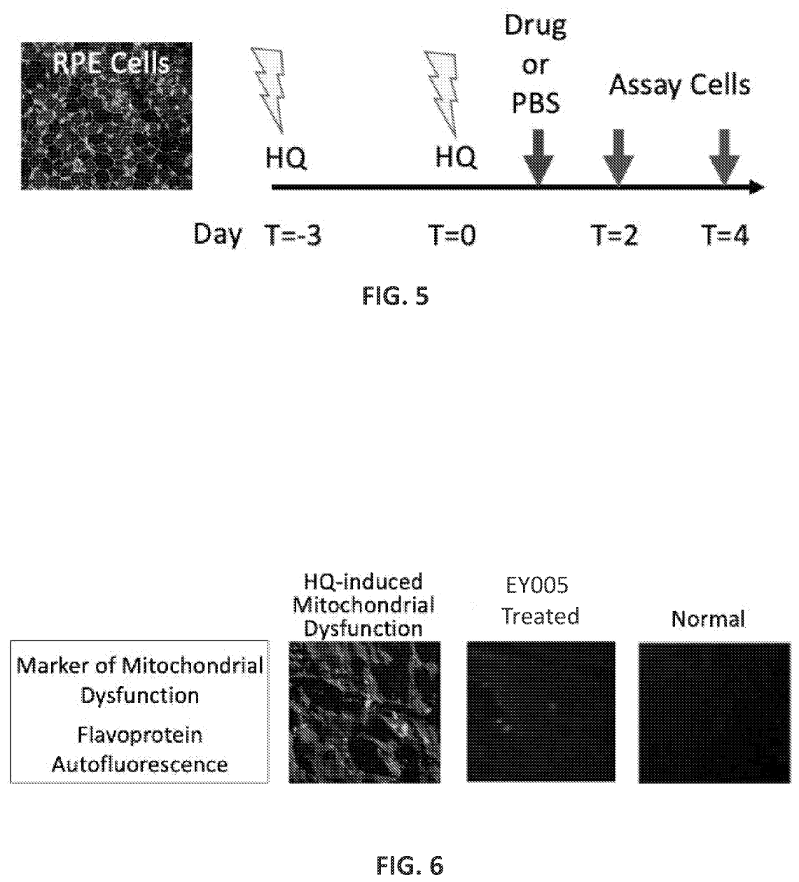

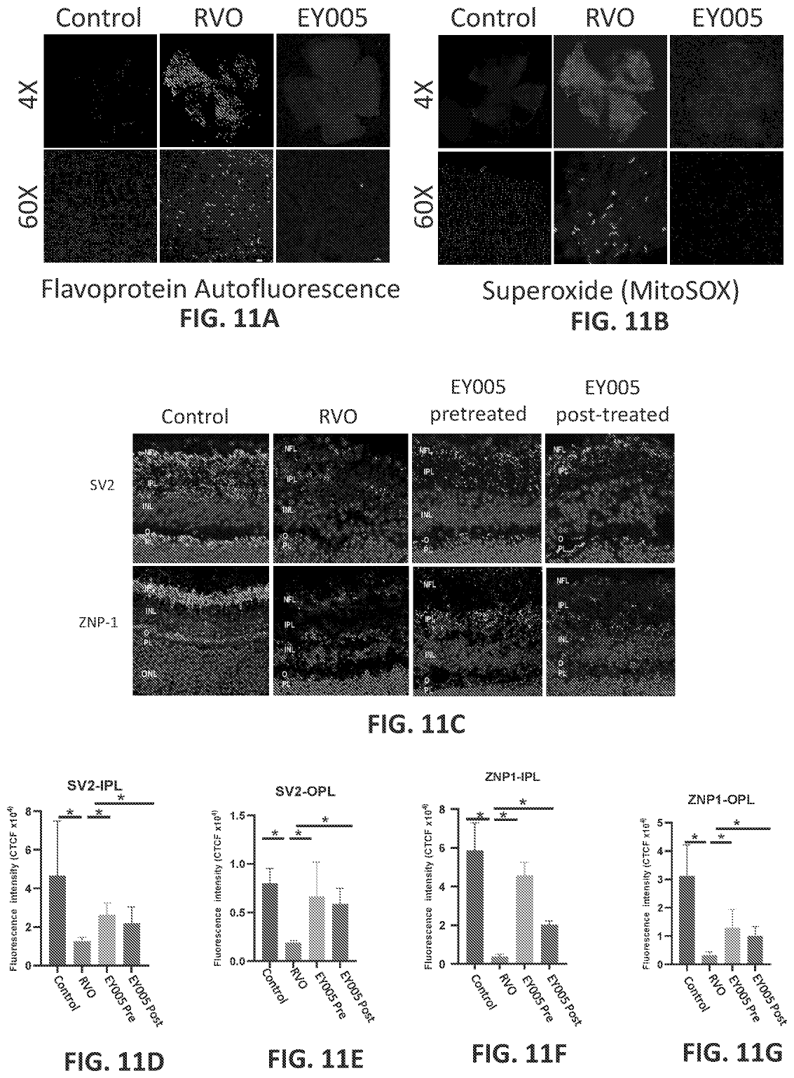

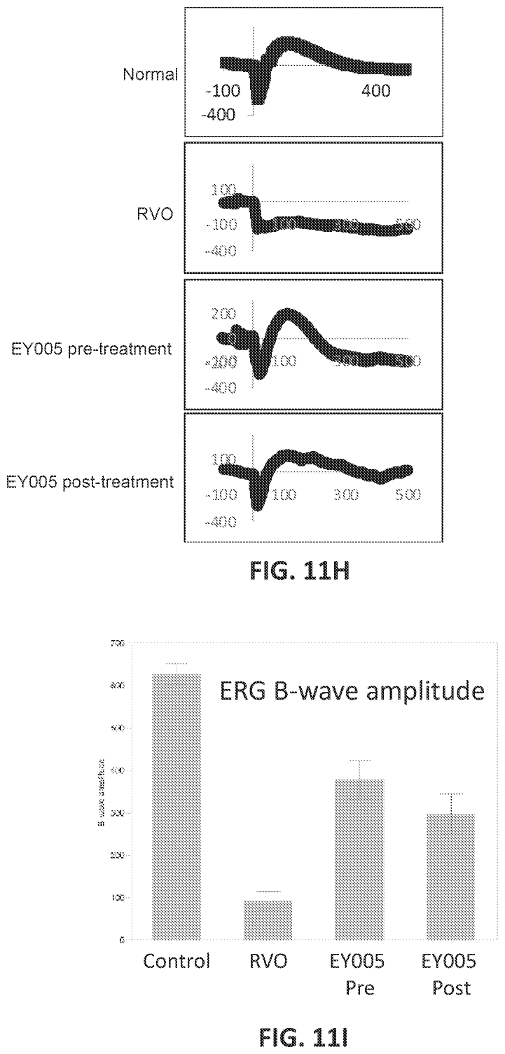

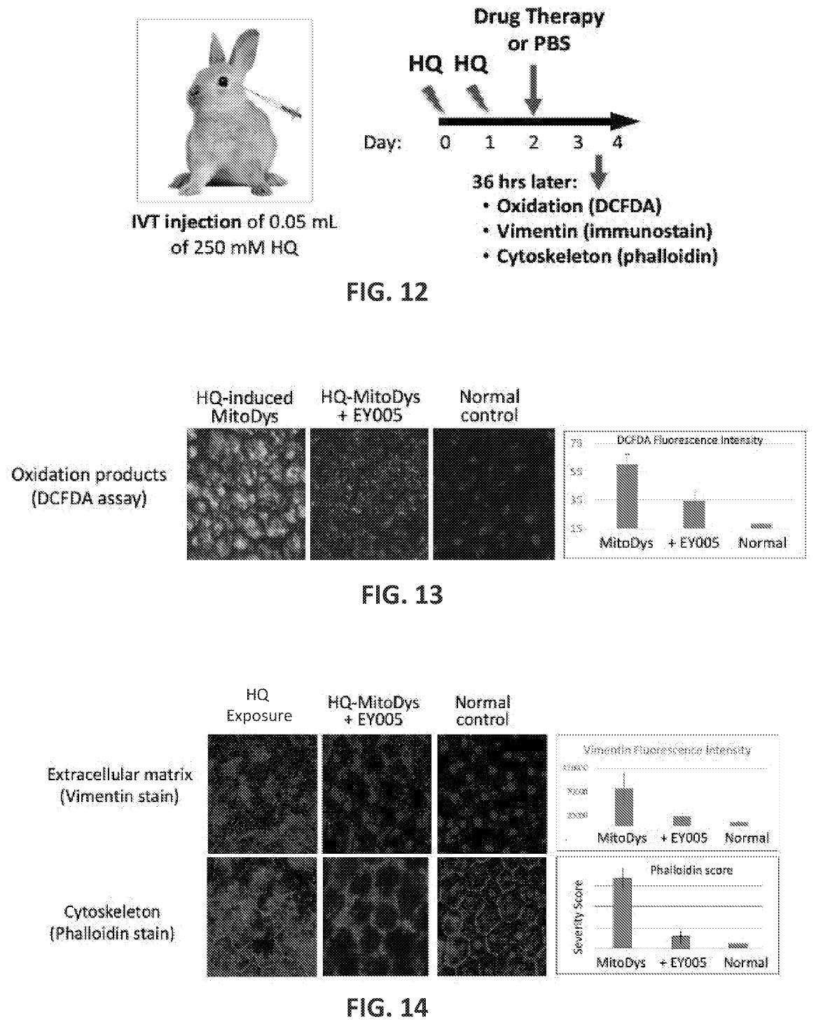

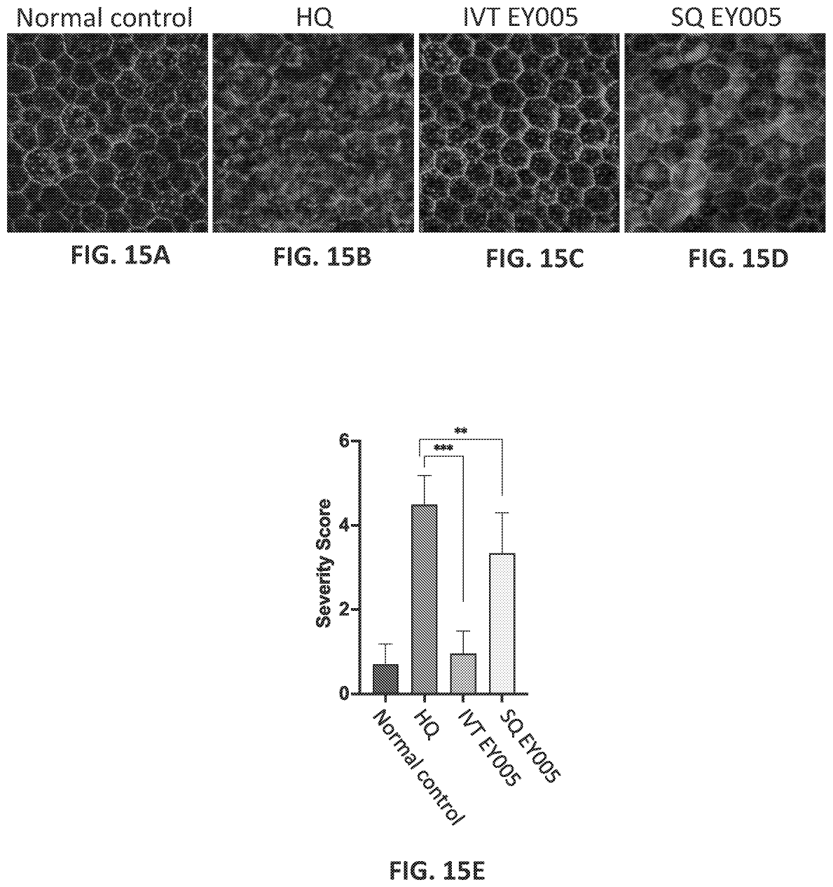

A previously undescribed member of the MTT class is H-d-Arg-DMT-Lys-Phe (FIG. 4A), termed herein as EY005, which differs from elamipretide in that the amino acid in the fourth position (i.e., C-terminus) is a phenylalanine rather than phenylalanine amide. EY005 has not previously been evaluated. As described herein, EY005 is equipotent to elamipretide in cell culture systems of AMD (FIGS. 5-10) and is partially effective in treatment of mouse model of RVO when given systemically at a dose greater than the maximally tolerated dose in humans (allometrically scaled) (FIG. 11). However, when given at the maximal human SQ dose (allometrically scaled), EY005 was only partially effective (FIG. 15D) at treating a rabbit model of dry AMD (FIG. 12) in terms of reversing key pathologies. In contrast, an intravitreal injection of EY005 is significantly more effective at reversing pathologies (FIG. 15C). Therapeutic responses are consistent with pharmacokinetic data that demonstrate that intravitreal (IVT) dosing achieves therapeutic levels of MTT that are detectable for 4 days, in contrast to systemic dosing, which achieves therapeutic levels for only 4 hours of sustained tissue exposure. MTT may require constant, sustained exposure to their target to be effective, which is not achieved by the transient, intermittent exposure to MTT that occurs with once daily systemic administration.

Described herein are extended release drug delivery systems (“XRDDS”) for MTT, for IVT and other routes of ocular administration. Unfortunately, as a tetrapeptide, EY005, elamipretide and other members of the MTT class are not well suited for IVT or periocular (i.e., subconjunctival or sub-Tenon's) routes of administration in their native form, due to their small size and high aqueous solubility. Further, due to their physicochemical properties, MTT are poorly compatible with currently available ocular drug delivery technologies and prior to this work have not been successfully formulated in an established drug delivery system.

Thus, the XRDDS formulations of EY005 or other MTT described herein, which are formulated in a manner that achieves sustained release in the eye and provides continuous exposure to predictable therapeutic levels in ocular tissues for a desired duration of treatment represent a significant improvement in compositions and treatments for mitochondrial dysfunction, particularly in the eye.

Described herein is a composition of EY005 or other MTTs manufactured as a prodrug, to make them compatible with a complexation-based XRDDS, for ocular use.

As used herein, “multiphasic” refers to a suspension in which the drug substance is present in more than one phase, including free drug, drug-drug aggregates, and most importantly, drug noncovalently bound to complexation agent particulates.

Extended release drug delivery systems (XRDDS) are devices, formulations or other systems used in the design, manufacture and administration of specific drug substances in a manner that regulates the drug release kinetics optimized for a specific therapeutic goal for a particular route of administration.

MTTs are four amino acid peptides comprising aromatic alternating with cationic amino acids. Typical examples of MTTs are listed in Table 1, below (SEQ ID NOs. 1-635). A useful MTT for treatment of ocular disease that can also serve to form an MTT-prodrug for formulation in the XRDDS is EY005, H-d-Arg-DMT-Lys-Phe, because it has a carboxylic acid in the fourth amino acid group, facilitating a covalent linkage to a conjugation moiety (FIG. 4B).

Herein, we disclose a XRDDS that has been optimized for MTT-prodrug in and around the eye.

As described herein, a multiphasic colloidal suspension incorporates MTT-prodrug as the drug substance. Formulation of MTT-prodrug into a multiphasic colloidal suspension forms a complexation-based XRDDS, referred to herein as Mito XR or MTT-prodrug multiphasic colloidal suspension (and/or may be an implant or part of an implant). Mito XR is a flowable viscous liquid that can be administered by intravitreal (IVT) or periocular routes to produce sustained release of therapeutic levels of active MTT drug within ocular tissues for desired duration (1 to 12 months), for the treatment of acquired and hereditary mitochondrial diseases of the eye.

More specifically, the compositions and methods described herein includes mitochondria-targeted tetrapeptide (MTT)-prodrugs, formed by a cleavable covalent linkage to a conjugation moiety chosen from one of five classes of chemical substances specifically chosen for their ability to form noncovalent complexes with one of six chemical substances that are not previously known to serve as complexation agents for MTT-prodrugs, especially when formulated as irregularly shaped particulates. The noncovalent interaction between MTT-prodrug and complexation agent results in formation of MTT-prodrug-complex particulates.

The present complexation-based XRDDS is a multiphasic colloidal suspension comprising one or more MTT-prodrug-complex particulates, admixed with a dispersal medium selected from one of four hydrophobic oils that were not previously known to form a stable multiphasic colloidal suspension with MTT-prodrug-complex particulates.

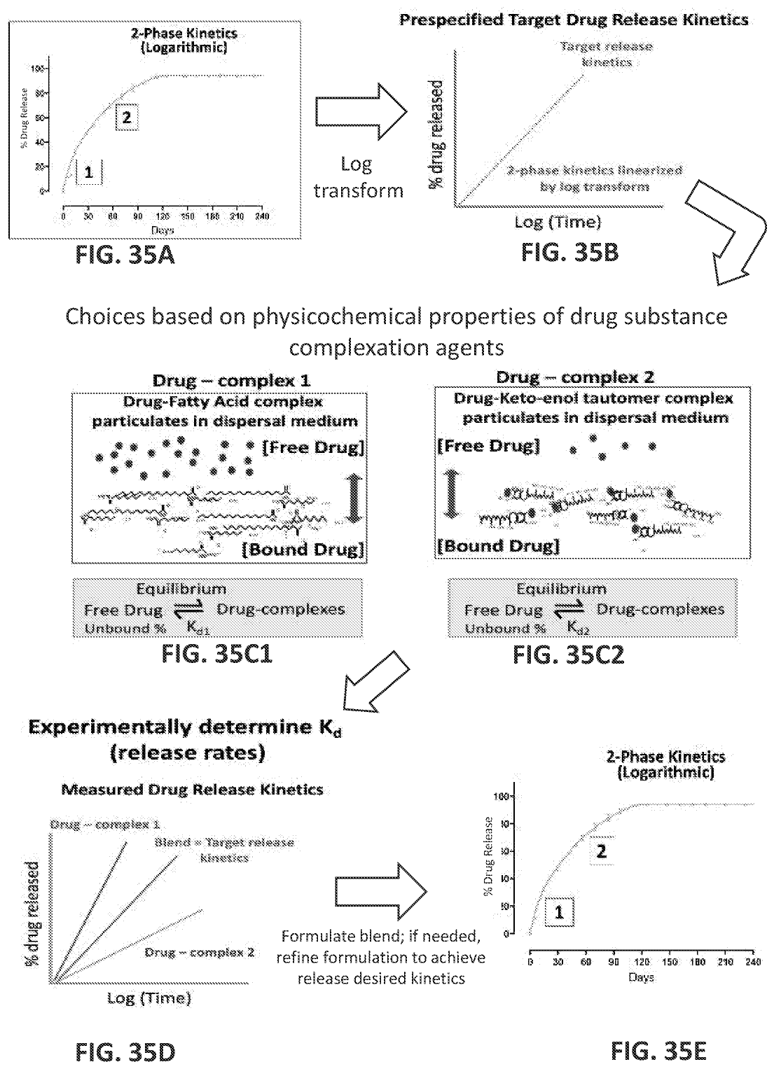

Also disclosed herein, the release kinetics of this complexation-based XRDDS can be designed and manufactured based upon knowledge of the unbound-bound fraction (Kd) of MTT-prodrug for each MTT-prodrug-complex particulate within a specific dispersal medium. Multiple different MTT-prodrug-complex particulate pairs, each with different Kd, can be combined in specific ratio and concentration to achieve a specific unbound, or free, concentration of MTT-prodrug within the dispersal medium of the multiphasic colloidal suspension, which will in turn determine the kinetics of release of MTT-prodrug from the implant into the ocular physiologic environment (FIGS. 35A-35E).

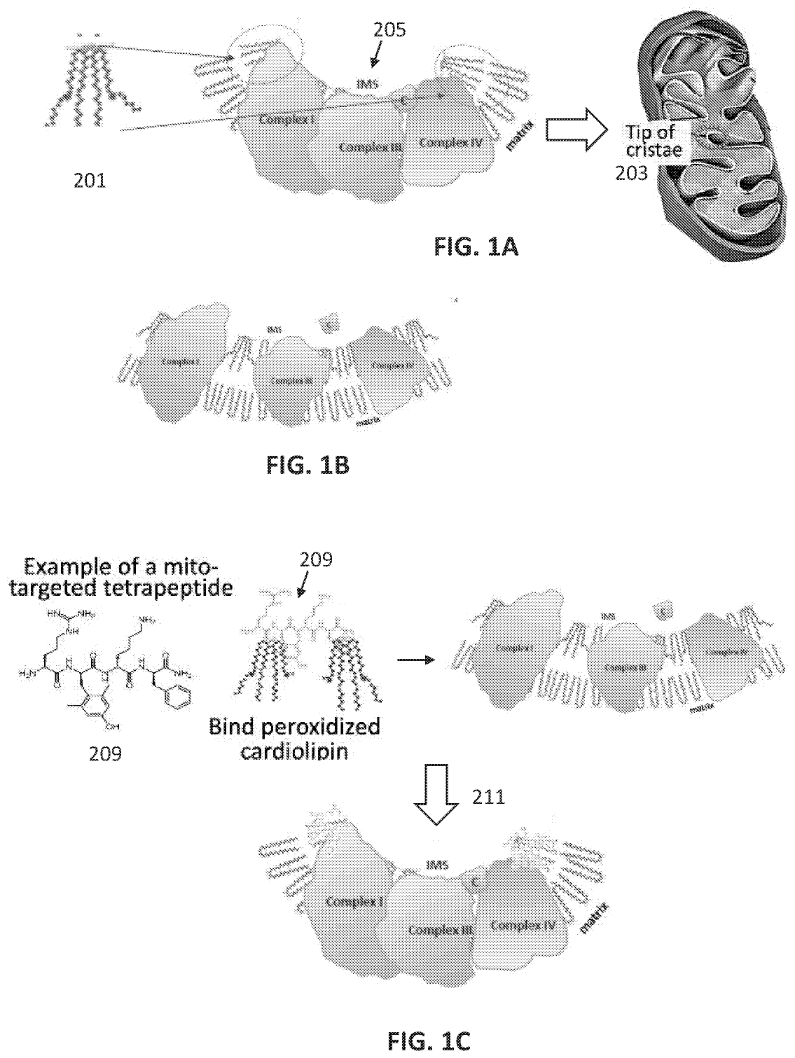

Mitochondrial dysfunction is characterized by decreased cellular bioenergetics (i.e., diminished adenosine triphosphate (ATP) production), increased superoxide, loss of calcium regulation, and other alterations. Irrespective of triggering factors (genetics, age, environmental), the final common pathway for mitochondrial dysfunction is peroxidation of cardiolipin, a unique dimeric phospholipid at the mitochondrial cristae (FIGS. 1A-1B). Cardiolipin's physicochemical properties (pyramidal shape, stiffness) are responsible for maintaining the distinctive “hairpin turn” of the cristae tip region, which in turns maintains the close approximation of the electron transport chain complexes necessary for ATP production and prevention of superoxide leakage (FIG. 1A). Upon peroxidation of cardiolipin, the cristae tips flatten, and electron transport chain complexes separate, leading to loss of ATP production and excess superoxide formation (FIG. 1B). Although various cardiolipin repair processes exist, these perform poorly in severely dysfunctional mitochondria.

A class of mitochondrial-targeted tetrapeptides (MTT) was serendipitously discovered by Hazel Szeto in 2000 while screening for peptides with opioid activity. It was subsequently discovered that specific tetrapeptides localized to mitochondria and further investigation revealed that functional tetrapeptides of this class require two cationic amino acids (which facilitate mitochondrial uptake) alternating with two aromatic amino acids, which facilitate binding to peroxidized cardiolipin, restoring its normal physicochemical properties, (FIG. 1C), which in turn restores close approximation of electron transport chain complexes and improves mitochondrial function.

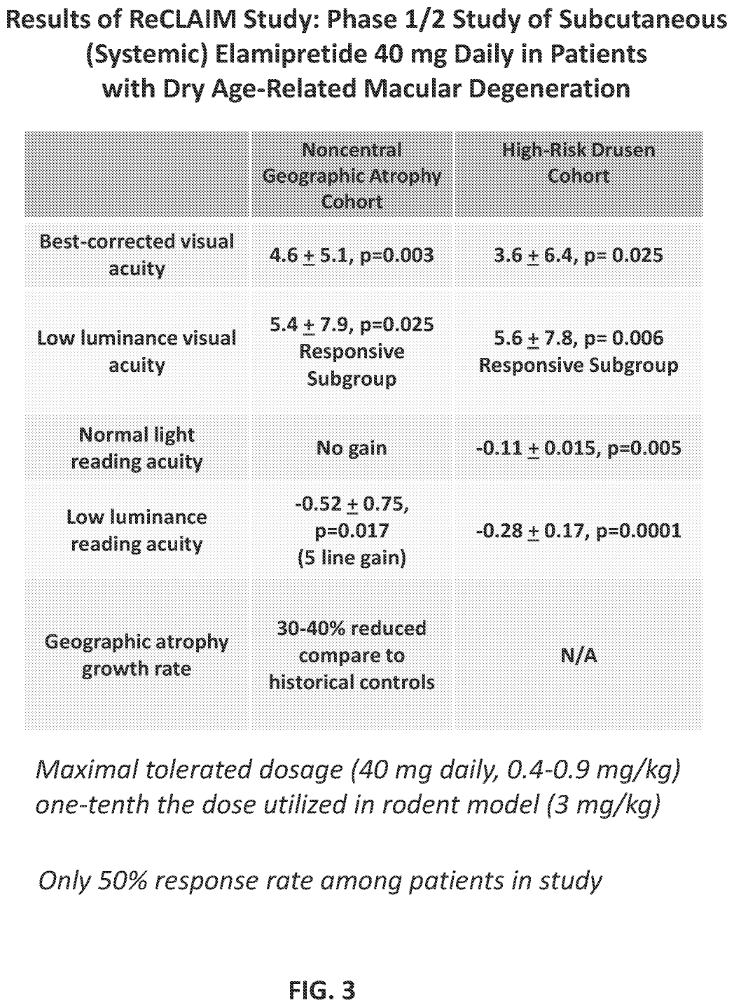

For example, elamipretide (formerly SS-31 or MTP-131) is a member of this previously discovered class of mitochondrial-targeted tetrapeptides, which has been studied clinical trials for mitochondrial disorders, including Barth Syndrome, a rare genetic mitochondrial disorder in children, and primary mitochondrial myopathy. In clinical trials and clinical application, elamipretide must be given as a daily subcutaneous (SQ) injection (FIG. 3).

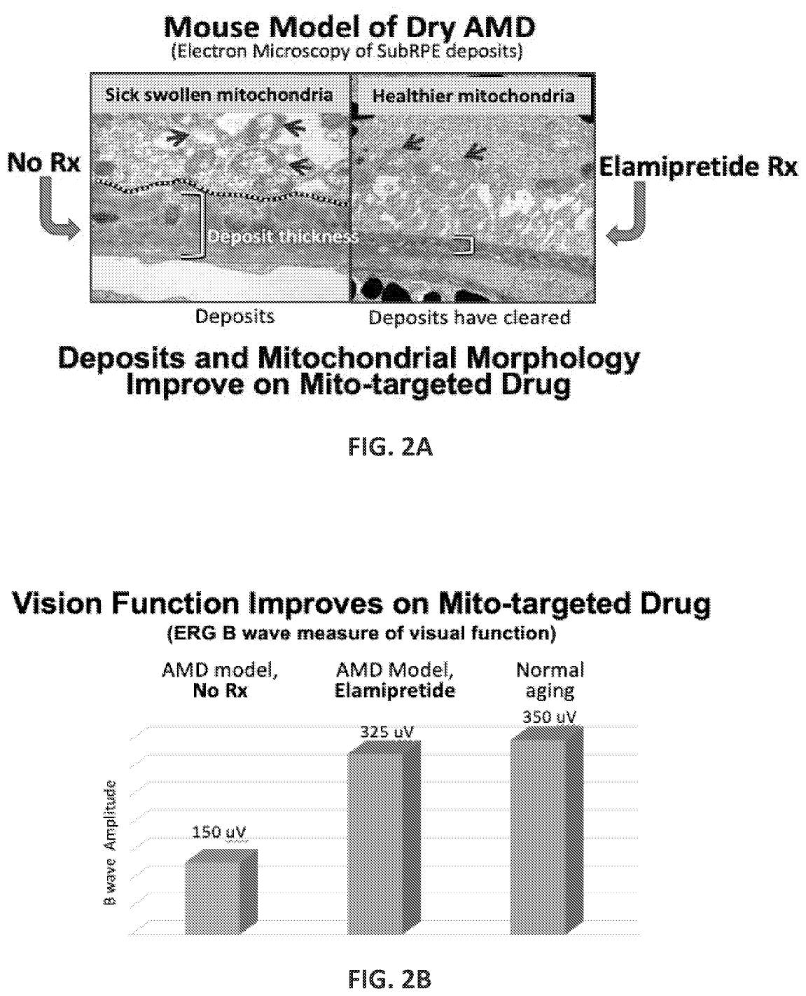

Systemically administered elamipretide has been studied in preclinical in vitro and in vivo models of dry AMD; it has been shown to be potent for reversal of mitochondrial dysfunction in RPE cell culture and in mouse models of dry AMD pathobiology, especially the local periocular hydroquinone-induced model and the ApoE4+high-fat diet model (FIGS. 2A-2B).

Intravitreal delivery of MTT is desirable to achieve sustained, high, and efficacious vitreous and retina drug levels. In their unmodified form, MTT have an intravitreal (IVT) half-life that is approximately 5-6 hours, with clearance from the eye in a few days. This would require, at a minimum, weekly IVT injections, which is impractical and not tenable as a treatment approach for ocular disease.

Furthermore, the small size of MTT and their high water solubility render them incompatible with most commercial drug delivery systems.

Novel therapeutic compounds described herein are referred to as “mitochondria-targeted tetrapeptide (MTT)-prodrugs.” MTT-prodrugs are comprising an active agent in the form of an MTT, a tetrapeptide with alternating cationic and aromatic residues, that reverses existing mitochondrial dysfunction and prevents further mitochondrial dysfunction and that is covalently linked via cleavable bond (e.g., ester bond) to one of five classes of inactive conjugation moieties.

The compositions and methods described herein may include a mitochondrial-targeted tetrapeptide (MTT), covalently linked to a conjugation moiety selected for its ability to form noncovalent reversible interactions with particulate complexation agents, optimizing its physicochemical properties for incorporation into the multiphasic colloidal embodiments. Also envisioned are other embodiments in which the conjugation moiety was chosen to alter the physicochemical properties of MTT, including size, charge, solubility, and physicochemical interaction with vehicles, and other properties that may facilitate formulation of MTT-prodrugs in other kinds of ophthalmic drug delivery systems.

One class of MTT-prodrugs described herein are those with covalently linked conjugation moiety specifically chosen for their capacity to form noncovalent complexes with one of five classes of complexation agents.

The MTT-prodrugs are compounds that may be products of condensation or esterification reactions, of formulas (I) or (II):

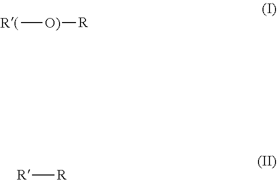

where R′ is an MTT, selected from among those with alternating cationic and aromatic amino acids listed in Table 1 (and SEQ ID NOs. 1-635), in which the C-terminal amino acid in the fourth position is covalently linked to conjugation moiety R, selected from among one of the following five classes of chemical substances: a C4-C30 lipid moiety, an C4-C30 straight-chain or branched aliphatic moiety, a 2-mer to 30-mer peptide moiety, a pegylated moiety, or a carbohydrate moiety.

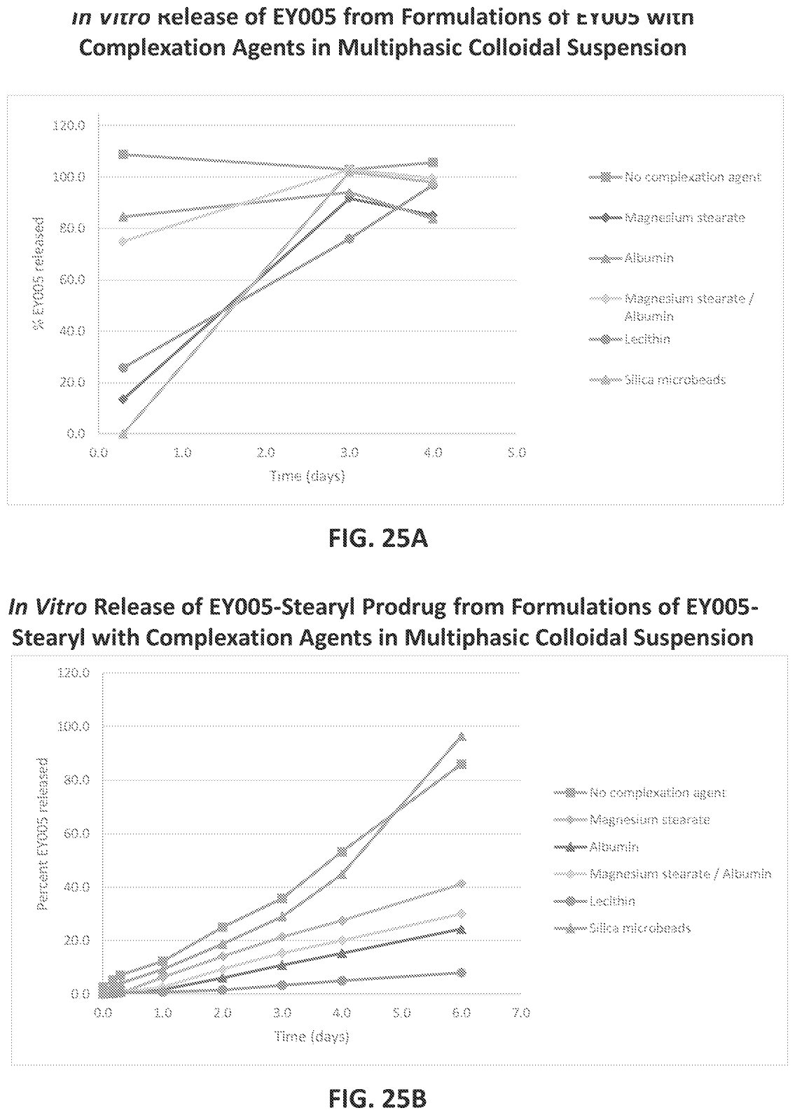

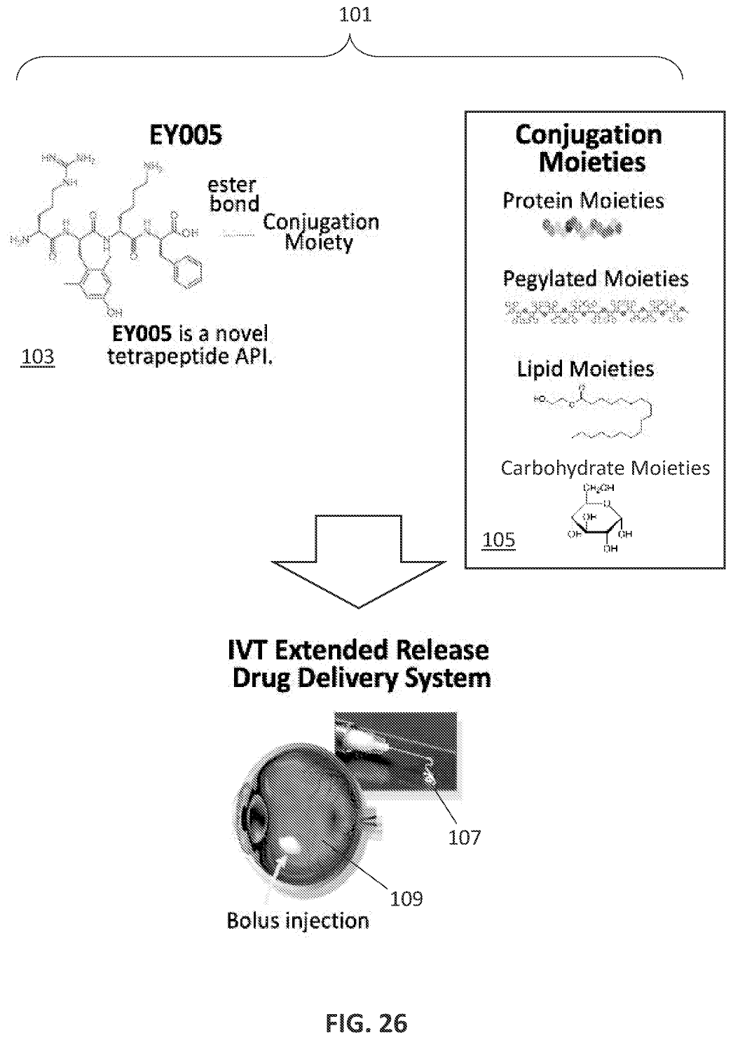

Prodrugs are pharmacologically inactive chemical modifications of the active pharmaceutical ingredient (API). Prodrugs are metabolized within the host either by tissue enzymes or by hydrolysis into the free API and the inactive conjugation moiety. Prodrugs are generally used to modify the API's physicochemical properties to improve absorption, bioavailability, or pharmacokinetics (PK). However, in the compositions and methods described herein, the purpose of the prodrug strategy is to optimize the drug's physicochemical properties for compatibility with the complexation-based extended release drug delivery system (XRDDS). This provides the mitochondrial targeted tetrapeptide drug with a slower release rate than could otherwise be achieved with non-prodrug forms (FIGS. 25, 27, and 29).

These prodrugs contain a bioactive molecule (ranging in length from 4 amino acids to 30 amino acids) that contains a tetrapeptide motif of alternating cationic and aromatic peptides, and with either a —OH (hydroxyl) or —COOH (carboxylic acid) group in the 4th position of the tetrapeptide motif to facilitate a covalent linkage to conjugation moiety. Table 1, below, is a table listing potential mitochondrial-targeting tetrapeptides that could be produced as prodrugs. Any of these mitochondrial-targeted tetrapeptides may be used as part of prodrugs (i.e., mitochondrial targeted extended release compounds) as described herein.

TABLE 1

| Amino | Amino | Amino | Amino | |
|---|---|---|---|---|
| Acid | Acid | Acid | Acid | |
| Position 1 | Position 2 | Position 3 | Position 4 | SEQ ID No. |
| d-Arg | 2′6′Dmt | Lys | Phe | SEQ ID No. 1 |
| d-Arg | 3′5′Dmt | Lys | Phe | SEQ ID No. 2 |
| Tyr | Lys | Phe | Dap | SEQ ID No. 3 |
| Tyr | Lys | Phe | Arg | SEQ ID No. 4 |
| Tyr | Lys | Phe | Lys | SEQ ID No. 5 |
| Tyr | Lys | Phe | Orn | SEQ ID No. 6 |
| Tyr | Lys | Phe | Dab | SEQ ID No. 7 |
| 2′6′Dmt | Lys | Phe | Dap | SEQ ID No. 8 |
| 2′6′Dmt | Lys | Phe | Arg | SEQ ID No. 9 |
| 2′6′Dmt | Lys | Phe | Lys | SEQ ID No. 10 |
| 2′6′Dmt | Lys | Phe | Orn | SEQ ID No. 11 |
| 2′6′Dmt | Lys | Phe | Dab | SEQ ID No. 12 |
| 3′5′Dmt | Lys | Phe | Dap | SEQ ID No. 13 |
| 3′5′Dmt | Lys | Phe | Arg | SEQ ID No. 14 |
| 3′5′Dmt | Lys | Phe | Lys | SEQ ID No. 15 |
| 3′5′Dmt | Lys | Phe | Orn | SEQ ID No. 16 |
| 3′5′Dmt | Lys | Phe | Dab | SEQ ID No. 17 |
| Mmt | Lys | Phe | Dap | SEQ ID No. 18 |
| Mmt | Lys | Phe | Arg | SEQ ID No. 19 |
| Mmt | Lys | Phe | Lys | SEQ ID No. 20 |
| Mmt | Lys | Phe | Orn | SEQ ID No. 21 |
| Mmt | Lys | Phe | Dab | SEQ ID No. 22 |
| Hmt | Lys | Phe | Dap | SEQ ID No. 23 |
| Hmt | Lys | Phe | Arg | SEQ ID No. 24 |
| Hmt | Lys | Phe | Lys | SEQ ID No. 25 |
| Hmt | Lys | Phe | Orn | SEQ ID No. 26 |
| Hmt | Lys | Phe | Dab | SEQ ID No. 27 |
| Tmt | Lys | Phe | Dap | SEQ ID No. 28 |
| Tmt | Lys | Phe | Arg | SEQ ID No. 29 |
| Tmt | Lys | Phe | Lys | SEQ ID No. 30 |
| Tmt | Lys | Phe | Orn | SEQ ID No. 31 |
| Tmt | Lys | Phe | Dab | SEQ ID No. 32 |
| Phe | Lys | Phe | Dap | SEQ ID No. 33 |
| Phe | Lys | Phe | Arg | SEQ ID No. 34 |
| Phe | Lys | Phe | Lys | SEQ ID No. 35 |
| Phe | Lys | Phe | Orn | SEQ ID No. 36 |
| Phe | Lys | Phe | Dab | SEQ ID No. 37 |
| Tyr | Arg | Phe | Dap | SEQ ID No. 38 |
| Tyr | Arg | Phe | Arg | SEQ ID No. 39 |
| Tyr | Arg | Phe | Lys | SEQ ID No. 40 |
| Tyr | Arg | Phe | Orn | SEQ ID No. 41 |
| Tyr | Arg | Phe | Dab | SEQ ID No. 42 |
| 2′6′Dmt | Arg | Phe | Dap | SEQ ID No. 43 |
| 2′6′Dmt | Arg | Phe | Arg | SEQ ID No. 44 |
| 2′6′Dmt | Arg | Phe | Lys | SEQ ID No. 45 |
| 2′6′Dmt | Arg | Phe | Orn | SEQ ID No. 46 |
| 2′6′Dmt | Arg | Phe | Dab | SEQ ID No. 47 |
| 3′5′Dmt | Arg | Phe | Dap | SEQ ID No. 48 |
| 3′5′Dmt | Arg | Phe | Arg | SEQ ID No. 49 |
| 3′5′Dmt | Arg | Phe | Lys | SEQ ID No. 50 |
| 3′5′Dmt | Arg | Phe | Orn | SEQ ID No. 51 |
| 3′5′Dmt | Arg | Phe | Dab | SEQ ID No. 52 |
| Mmt | Arg | Phe | Dap | SEQ ID No. 53 |
| Mmt | Arg | Phe | Arg | SEQ ID No. 54 |
| Mmt | Arg | Phe | Lys | SEQ ID No. 55 |
| Mmt | Arg | Phe | Orn | SEQ ID No. 56 |
| Mmt | Arg | Phe | Dab | SEQ ID No. 57 |
| Hmt | Arg | Phe | Dap | SEQ ID No. 58 |
| Hmt | Arg | Phe | Arg | SEQ ID No. 59 |
| Hmt | Arg | Phe | Lys | SEQ ID No. 60 |
| Hmt | Arg | Phe | Orn | SEQ ID No. 61 |
| Hmt | Arg | Phe | Dab | SEQ ID No. 62 |
| Tmt | Arg | Phe | Dap | SEQ ID No. 63 |
| Tmt | Arg | Phe | Arg | SEQ ID No. 64 |
| Tmt | Arg | Phe | Lys | SEQ ID No. 65 |
| Tmt | Arg | Phe | Orn | SEQ ID No. 66 |
| Tmt | Arg | Phe | Dab | SEQ ID No. 67 |
| Phe | Arg | Phe | Dap | SEQ ID No. 68 |
| Phe | Arg | Phe | Arg | SEQ ID No. 69 |
| Phe | Arg | Phe | Lys | SEQ ID No. 70 |
| Phe | Arg | Phe | Orn | SEQ ID No. 71 |
| Phe | Arg | Phe | Dab | SEQ ID No. 72 |
| Tyr | Dap | Phe | D-Lys | SEQ ID No. 73 |
| Tyr | Arg | Phe | D-Lys | SEQ ID No. 74 |
| Tyr | Lys | Phe | D-Lys | SEQ ID No. 75 |
| Tyr | Orn | Phe | D-Lys | SEQ ID No. 76 |
| Tyr | Dab | Phe | D-Lys | SEQ ID No. 77 |
| 2′6′Dmt | Dap | Phe | D-Lys | SEQ ID No. 78 |
| 2′6′Dmt | Arg | Phe | D-Lys | SEQ ID No. 79 |
| 2′6′Dmt | Lys | Phe | D-Lys | SEQ ID No. 80 |
| 2′6′Dmt | Orn | Phe | D-Lys | SEQ ID No. 81 |
| 2′6′Dmt | Dab | Phe | D-Lys | SEQ ID No. 82 |
| 3′5′Dmt | Dap | Phe | D-Lys | SEQ ID No. 83 |
| 3′5′Dmt | Arg | Phe | D-Lys | SEQ ID No. 84 |
| 3′5′Dmt | Lys | Phe | D-Lys | SEQ ID No. 85 |
| 3′5′Dmt | Orn | Phe | D-Lys | SEQ ID No. 86 |
| 3′5′Dmt | Dab | Phe | D-Lys | SEQ ID No. 87 |
| Mmt | Dap | Phe | D-Lys | SEQ ID No. 88 |
| Mmt | Arg | Phe | D-Lys | SEQ ID No. 89 |
| Mmt | Lys | Phe | D-Lys | SEQ ID No. 90 |
| Mmt | Orn | Phe | D-Lys | SEQ ID No. 91 |
| Mmt | Dab | Phe | D-Lys | SEQ ID No. 92 |
| Hmt | Dap | Phe | D-Lys | SEQ ID No. 93 |
| Hmt | Arg | Phe | D-Lys | SEQ ID No. 94 |
| Hmt | Lys | Phe | D-Lys | SEQ ID No. 95 |
| Hmt | Orn | Phe | D-Lys | SEQ ID No. 96 |
| Hmt | Dab | Phe | D-Lys | SEQ ID No. 97 |
| Tmt | Dap | Phe | D-Lys | SEQ ID No. 98 |
| Tmt | Arg | Phe | D-Lys | SEQ ID No. 99 |
| Tmt | Lys | Phe | D-Lys | SEQ ID No. 100 |
| Tmt | Orn | Phe | D-Lys | SEQ ID No. 101 |
| Tmt | Dab | Phe | D-Lys | SEQ ID No. 102 |
| Phe | Dap | Phe | D-Lys | SEQ ID No. 103 |
| Phe | Arg | Phe | D-Lys | SEQ ID No. 104 |
| Phe | Lys | Phe | D-Lys | SEQ ID No. 105 |
| Phe | Orn | Phe | D-Lys | SEQ ID No. 106 |
| Phe | Dab | Phe | D-Lys | SEQ ID No. 107 |
| Tyr | Dap | Phe | Lys | SEQ ID No. 108 |
| Tyr | Arg | Phe | Lys | SEQ ID No. 109 |
| Tyr | Lys | Phe | Lys | SEQ ID No. 110 |
| Tyr | Orn | Phe | Lys | SEQ ID No. 111 |
| Tyr | Dab | Phe | Lys | SEQ ID No. 112 |
| 2′6′Dmt | Dap | Phe | Lys | SEQ ID No. 113 |
| 2′6′Dmt | Arg | Phe | Lys | SEQ ID No. 114 |
| 2′6′Dmt | Lys | Phe | Lys | SEQ ID No. 115 |
| 2′6′Dmt | Orn | Phe | Lys | SEQ ID No. 116 |
| 2′6′Dmt | Dab | Phe | Lys | SEQ ID No. 117 |
| 3′5′Dmt | Dap | Phe | Lys | SEQ ID No. 118 |
| 3′5′Dmt | Arg | Phe | Lys | SEQ ID No. 119 |
| 3′5′Dmt | Lys | Phe | Lys | SEQ ID No. 120 |
| 3′5′Dmt | Orn | Phe | Lys | SEQ ID No. 121 |
| 3′5′Dmt | Dab | Phe | Lys | SEQ ID No. 122 |
| Mmt | Dap | Phe | Lys | SEQ ID No. 123 |
| Mmt | Arg | Phe | Lys | SEQ ID No. 124 |
| Mmt | Lys | Phe | Lys | SEQ ID No. 125 |
| Mmt | Orn | Phe | Lys | SEQ ID No. 126 |
| Mmt | Dab | Phe | Lys | SEQ ID No. 127 |
| Hmt | Dap | Phe | Lys | SEQ ID No. 128 |
| Hmt | Arg | Phe | Lys | SEQ ID No. 129 |
| Hmt | Lys | Phe | Lys | SEQ ID No. 130 |
| Hmt | Orn | Phe | Lys | SEQ ID No. 131 |
| Hmt | Dab | Phe | Lys | SEQ ID No. 132 |
| Tmt | Dap | Phe | Lys | SEQ ID No. 133 |
| Tmt | Arg | Phe | Lys | SEQ ID No. 134 |
| Tmt | Lys | Phe | Lys | SEQ ID No. 135 |
| Tmt | Orn | Phe | Lys | SEQ ID No. 136 |
| Tmt | Dab | Phe | Lys | SEQ ID No. 137 |
| Phe | Dap | Phe | Lys | SEQ ID No. 138 |
| Phe | Arg | Phe | Lys | SEQ ID No. 139 |
| Phe | Lys | Phe | Lys | SEQ ID No. 140 |
| Phe | Orn | Phe | Lys | SEQ ID No. 141 |
| Phe | Dab | Phe | Lys | SEQ ID No. 142 |
| Tyr | Dap | Phe | Arg | SEQ ID No. 143 |
| Tyr | Arg | Phe | Arg | SEQ ID No. 144 |
| Tyr | Lys | Phe | Arg | SEQ ID No. 145 |
| Tyr | Orn | Phe | Arg | SEQ ID No. 146 |
| Tyr | Dab | Phe | Arg | SEQ ID No. 147 |
| 2′6′Dmt | Dap | Phe | Arg | SEQ ID No. 148 |
| 2′6′Dmt | Arg | Phe | Arg | SEQ ID No. 149 |
| 2′6′Dmt | Lys | Phe | Arg | SEQ ID No. 150 |
| 2′6′Dmt | Orn | Phe | Arg | SEQ ID No. 151 |
| 2′6′Dmt | Dab | Phe | Arg | SEQ ID No. 152 |
| 3′5′Dmt | Dap | Phe | Arg | SEQ ID No. 153 |
| 3′5′Dmt | Arg | Phe | Arg | SEQ ID No. 154 |
| 3′5′Dmt | Lys | Phe | Arg | SEQ ID No. 155 |
| 3′5′Dmt | Orn | Phe | Arg | SEQ ID No. 156 |
| 3′5′Dmt | Dab | Phe | Arg | SEQ ID No. 157 |
| Mmt | Dap | Phe | Arg | SEQ ID No. 158 |
| Mmt | Arg | Phe | Arg | SEQ ID No. 159 |
| Mmt | Lys | Phe | Arg | SEQ ID No. 160 |
| Mmt | Orn | Phe | Arg | SEQ ID No. 161 |
| Mmt | Dab | Phe | Arg | SEQ ID No. 162 |
| Hmt | Dap | Phe | Arg | SEQ ID No. 163 |
| Hmt | Arg | Phe | Arg | SEQ ID No. 164 |
| Hmt | Lys | Phe | Arg | SEQ ID No. 165 |
| Hmt | Orn | Phe | Arg | SEQ ID No. 166 |
| Hmt | Dab | Phe | Arg | SEQ ID No. 167 |
| Tmt | Dap | Phe | Arg | SEQ ID No. 168 |
| Tmt | Arg | Phe | Arg | SEQ ID No. 169 |
| Tmt | Lys | Phe | Arg | SEQ ID No. 170 |
| Tmt | Orn | Phe | Arg | SEQ ID No. 171 |
| Tmt | Dab | Phe | Arg | SEQ ID No. 172 |
| Phe | Dap | Phe | Arg | SEQ ID No. 173 |
| Phe | Arg | Phe | Arg | SEQ ID No. 174 |
| Phe | Lys | Phe | Arg | SEQ ID No. 175 |
| Phe | Orn | Phe | Arg | SEQ ID No. 176 |
| Phe | Dab | Phe | Arg | SEQ ID No. 177 |
| Lys | Phe | Dap | Tyr | SEQ ID No. 178 |
| Lys | Phe | Arg | Tyr | SEQ ID No. 179 |
| Lys | Phe | Lys | Tyr | SEQ ID No. 180 |
| Lys | Phe | Orn | Tyr | SEQ ID No. 181 |
| Lys | Phe | Dab | Tyr | SEQ ID No. 182 |
| Lys | Phe | Dap | 2′6′Dmt | SEQ ID No. 183 |
| Lys | Phe | Arg | 2′6′Dmt | SEQ ID No. 184 |
| Lys | Phe | Lys | 2′6′Dmt | SEQ ID No. 185 |
| Lys | Phe | Orn | 2′6′Dmt | SEQ ID No. 186 |
| Lys | Phe | Dab | 2′6′Dmt | SEQ ID No. 187 |
| Lys | Phe | Dap | 3′5′Dmt | SEQ ID No. 188 |
| Lys | Phe | Arg | 3′5′Dmt | SEQ ID No. 189 |
| Lys | Phe | Lys | 3′5′Dmt | SEQ ID No. 190 |
| Lys | Phe | Orn | 3′5′Dmt | SEQ ID No. 191 |
| Lys | Phe | Dab | 3′5′Dmt | SEQ ID No. 192 |
| Lys | Phe | Dap | Mmt | SEQ ID No. 193 |
| Lys | Phe | Arg | Mmt | SEQ ID No. 194 |
| Lys | Phe | Lys | Mmt | SEQ ID No. 195 |
| Lys | Phe | Orn | Mmt | SEQ ID No. 196 |
| Lys | Phe | Dab | Mmt | SEQ ID No. 197 |
| Lys | Phe | Dap | Hmt | SEQ ID No. 198 |
| Lys | Phe | Arg | Hmt | SEQ ID No. 199 |
| Lys | Phe | Lys | Hmt | SEQ ID No. 200 |
| Lys | Phe | Orn | Hmt | SEQ ID No. 201 |
| Lys | Phe | Dab | Hmt | SEQ ID No. 202 |
| Lys | Phe | Dap | Tmt | SEQ ID No. 203 |
| Lys | Phe | Arg | Tmt | SEQ ID No. 204 |
| Lys | Phe | Lys | Tmt | SEQ ID No. 205 |
| Lys | Phe | Orn | Tmt | SEQ ID No. 206 |
| Lys | Phe | Dab | Tmt | SEQ ID No. 207 |
| Lys | Phe | Dap | Phe | SEQ ID No. 208 |
| Lys | Phe | Arg | Phe | SEQ ID No. 209 |
| Lys | Phe | Lys | Phe | SEQ ID No. 210 |
| Lys | Phe | Orn | Phe | SEQ ID No. 211 |
| Lys | Phe | Dab | Phe | SEQ ID No. 212 |
| Arg | Phe | Dap | Tyr | SEQ ID No. 213 |
| Arg | Phe | Arg | Tyr | SEQ ID No. 214 |
| Arg | Phe | Lys | Tyr | SEQ ID No. 215 |
| Arg | Phe | Orn | Tyr | SEQ ID No. 216 |
| Arg | Phe | Dab | Tyr | SEQ ID No. 217 |
| Arg | Phe | Dap | 2′6′Dmt | SEQ ID No. 218 |
| Arg | Phe | Arg | 2′6′Dmt | SEQ ID No. 219 |
| Arg | Phe | Lys | 2′6′Dmt | SEQ ID No. 220 |
| Arg | Phe | Orn | 2′6′Dmt | SEQ ID No. 221 |
| Arg | Phe | Dab | 2′6′Dmt | SEQ ID No. 222 |
| Arg | Phe | Dap | 3′5′Dmt | SEQ ID No. 223 |
| Arg | Phe | Arg | 3′5′Dmt | SEQ ID No. 224 |
| Arg | Phe | Lys | 3′5′Dmt | SEQ ID No. 225 |
| Arg | Phe | Orn | 3′5′Dmt | SEQ ID No. 226 |
| Arg | Phe | Dab | 3′5′Dmt | SEQ ID No. 227 |
| Arg | Phe | Dap | Mmt | SEQ ID No. 228 |
| Arg | Phe | Arg | Mmt | SEQ ID No. 229 |
| Arg | Phe | Lys | Mmt | SEQ ID No. 230 |
| Arg | Phe | Orn | Mmt | SEQ ID No. 231 |
| Arg | Phe | Dab | Mmt | SEQ ID No. 232 |
| Arg | Phe | Dap | Hmt | SEQ ID No. 233 |
| Arg | Phe | Arg | Hmt | SEQ ID No. 234 |
| Arg | Phe | Lys | Hmt | SEQ ID No. 235 |
| Arg | Phe | Orn | Hmt | SEQ ID No. 236 |
| Arg | Phe | Dab | Hmt | SEQ ID No. 237 |
| Arg | Phe | Dap | Tmt | SEQ ID No. 238 |
| Arg | Phe | Arg | Tmt | SEQ ID No. 239 |
| Arg | Phe | Lys | Tmt | SEQ ID No. 240 |
| Arg | Phe | Orn | Tmt | SEQ ID No. 241 |
| Arg | Phe | Dab | Tmt | SEQ ID No. 242 |
| Arg | Phe | Dap | Phe | SEQ ID No. 243 |
| Arg | Phe | Arg | Phe | SEQ ID No. 244 |
| Arg | Phe | Lys | Phe | SEQ ID No. 245 |
| Arg | Phe | Orn | Phe | SEQ ID No. 246 |
| Arg | Phe | Dab | Phe | SEQ ID No. 247 |
| Dap | Phe | Lys | Tyr | SEQ ID No. 248 |
| Arg | Phe | Lys | Tyr | SEQ ID No. 249 |
| Lys | Phe | Lys | Tyr | SEQ ID No. 250 |
| Orn | Phe | Lys | Tyr | SEQ ID No. 251 |
| Dab | Phe | Lys | Tyr | SEQ ID No. 252 |
| Dap | Phe | Lys | 2′6′Dmt | SEQ ID No. 253 |
| Arg | Phe | Lys | 2′6′Dmt | SEQ ID No. 254 |
| Lys | Phe | Lys | 2′6′Dmt | SEQ ID No. 255 |
| Orn | Phe | Lys | 2′6′Dmt | SEQ ID No. 256 |
| Dab | Phe | Lys | 2′6′Dmt | SEQ ID No. 257 |
| Dap | Phe | Lys | 3′5′Dmt | SEQ ID No. 258 |
| Arg | Phe | Lys | 3′5′Dmt | SEQ ID No. 259 |
| Lys | Phe | Lys | 3′5′Dmt | SEQ ID No. 260 |
| Orn | Phe | Lys | 3′5′Dmt | SEQ ID No. 261 |
| Dab | Phe | Lys | 3′5′Dmt | SEQ ID No. 262 |
| Dap | Phe | Lys | Mmt | SEQ ID No. 263 |
| Arg | Phe | Lys | Mmt | SEQ ID No. 264 |
| Lys | Phe | Lys | Mmt | SEQ ID No. 265 |
| Orn | Phe | Lys | Mmt | SEQ ID No. 266 |
| Dab | Phe | Lys | Mmt | SEQ ID No. 267 |
| Dap | Phe | Lys | Hmt | SEQ ID No. 268 |
| Arg | Phe | Lys | Hmt | SEQ ID No. 269 |
| Lys | Phe | Lys | Hmt | SEQ ID No. 270 |
| Orn | Phe | Lys | Hmt | SEQ ID No. 271 |
| Dab | Phe | Lys | Hmt | SEQ ID No. 272 |
| Dap | Phe | Lys | Tmt | SEQ ID No. 273 |
| Arg | Phe | Lys | Tmt | SEQ ID No. 274 |
| Lys | Phe | Lys | Tmt | SEQ ID No. 275 |
| Orn | Phe | Lys | Tmt | SEQ ID No. 276 |
| Dab | Phe | Lys | Tmt | SEQ ID No. 277 |
| Dap | Phe | Lys | Phe | SEQ ID No. 278 |
| Arg | Phe | Lys | Phe | SEQ ID No. 279 |
| Lys | Phe | Lys | Phe | SEQ ID No. 280 |
| Orn | Phe | Lys | Phe | SEQ ID No. 281 |
| Dab | Phe | Lys | Phe | SEQ ID No. 282 |
| Dap | Phe | Arg | Tyr | SEQ ID No. 283 |
| Arg | Phe | Arg | Tyr | SEQ ID No. 284 |
| Lys | Phe | Arg | Tyr | SEQ ID No. 285 |
| Orn | Phe | Arg | Tyr | SEQ ID No. 286 |
| Dab | Phe | Arg | Tyr | SEQ ID No. 287 |
| Dap | Phe | Arg | 2′6′Dmt | SEQ ID No. 288 |
| Arg | Phe | Arg | 2′6′Dmt | SEQ ID No. 289 |
| Lys | Phe | Arg | 2′6′Dmt | SEQ ID No. 290 |
| Orn | Phe | Arg | 2′6′Dmt | SEQ ID No. 291 |
| Dab | Phe | Arg | 2′6′Dmt | SEQ ID No. 292 |
| Dap | Phe | Arg | 3′5′Dmt | SEQ ID No. 293 |
| Arg | Phe | Arg | 3′5′Dmt | SEQ ID No. 294 |
| Lys | Phe | Arg | 3′5′Dmt | SEQ ID No. 295 |
| Orn | Phe | Arg | 3′5′Dmt | SEQ ID No. 296 |
| Dab | Phe | Arg | 3′5′Dmt | SEQ ID No. 297 |
| Dap | Phe | Arg | Mmt | SEQ ID No. 298 |
| Arg | Phe | Arg | Mmt | SEQ ID No. 299 |
| Lys | Phe | Arg | Mmt | SEQ ID No. 300 |
| Orn | Phe | Arg | Mmt | SEQ ID No. 301 |
| Dab | Phe | Arg | Mmt | SEQ ID No. 302 |
| Dap | Phe | Arg | Hmt | SEQ ID No. 303 |
| Arg | Phe | Arg | Hmt | SEQ ID No. 304 |
| Lys | Phe | Arg | Hmt | SEQ ID No. 305 |
| Orn | Phe | Arg | Hmt | SEQ ID No. 306 |
| Dab | Phe | Arg | Hmt | SEQ ID No. 307 |
| Dap | Phe | Arg | Tmt | SEQ ID No. 308 |
| Arg | Phe | Arg | Tmt | SEQ ID No. 309 |
| Lys | Phe | Arg | Tmt | SEQ ID No. 310 |
| Orn | Phe | Arg | Tmt | SEQ ID No. 311 |
| Dab | Phe | Arg | Tmt | SEQ ID No. 312 |
| Dap | Phe | Arg | Phe | SEQ ID No. 313 |
| Arg | Phe | Arg | Phe | SEQ ID No. 314 |
| Lys | Phe | Arg | Phe | SEQ ID No. 315 |
| Orn | Phe | Arg | Phe | SEQ ID No. 316 |
| Dab | Phe | Arg | Phe | SEQ ID No. 317 |
| Tyr | d-Arg | Phe | Lys | SEQ ID No. 318 |
| 2′6′Dmt | d-Arg | Phe | Lys | SEQ ID No. 319 |
| Phe | d-Arg | Phe | Lys | SEQ ID No. 320 |
| Tyr | Dap | Phe | Lys | SEQ ID No. 321 |
| Tyr | Arg | Phe | Lys | SEQ ID No. 322 |
| Tyr | Lys | Phe | Lys | SEQ ID No. 323 |
| Tyr | Orn | Phe | Lys | SEQ ID No. 324 |
| Tyr | Dab | Phe | Lys | SEQ ID No. 325 |
| 2′6′Dmt | Dap | Phe | Lys | SEQ ID No. 326 |
| 2′6′Dmt | Arg | Phe | Lys | SEQ ID No. 327 |
| 2′6′Dmt | Lys | Phe | Lys | SEQ ID No. 328 |
| 2′6′Dmt | Orn | Phe | Lys | SEQ ID No. 329 |
| 2′6′Dmt | Dab | Phe | Lys | SEQ ID No. 330 |
| 3′5′Dmt | Dap | Phe | Lys | SEQ ID No. 331 |
| 3′5′Dmt | Arg | Phe | Lys | SEQ ID No. 332 |
| 3′5′Dmt | Lys | Phe | Lys | SEQ ID No. 333 |
| 3′5′Dmt | Orn | Phe | Lys | SEQ ID No. 334 |
| 3′5′Dmt | Dab | Phe | Lys | SEQ ID No. 335 |
| Mmt | Dap | Phe | Lys | SEQ ID No. 336 |
| Mmt | Arg | Phe | Lys | SEQ ID No. 337 |
| Mmt | Lys | Phe | Lys | SEQ ID No. 338 |
| Mmt | Orn | Phe | Lys | SEQ ID No. 339 |
| Mmt | Dab | Phe | Lys | SEQ ID No. 340 |
| Hmt | Dap | Phe | Lys | SEQ ID No. 341 |
| Hmt | Arg | Phe | Lys | SEQ ID No. 342 |
| Hmt | Lys | Phe | Lys | SEQ ID No. 343 |
| Hmt | Orn | Phe | Lys | SEQ ID No. 344 |
| Hmt | Dab | Phe | Lys | SEQ ID No. 345 |
| Tmt | Dap | Phe | Lys | SEQ ID No. 346 |
| Tmt | Arg | Phe | Lys | SEQ ID No. 347 |
| Tmt | Lys | Phe | Lys | SEQ ID No. 348 |
| Tmt | Orn | Phe | Lys | SEQ ID No. 349 |
| Tmt | Dab | Phe | Lys | SEQ ID No. 350 |
| Phe | Dap | Phe | Lys | SEQ ID No. 351 |
| Phe | Arg | Phe | Lys | SEQ ID No. 352 |
| Phe | Lys | Phe | Lys | SEQ ID No. 353 |
| Phe | Orn | Phe | Lys | SEQ ID No. 354 |
| Phe | Dab | Phe | Lys | SEQ ID No. 355 |
| Tyr | Dap | Phe | Arg | SEQ ID No. 356 |
| Tyr | Arg | Phe | Arg | SEQ ID No. 357 |
| Tyr | Lys | Phe | Arg | SEQ ID No. 358 |
| Tyr | Orn | Phe | Arg | SEQ ID No. 359 |
| Tyr | Dab | Phe | Arg | SEQ ID No. 360 |
| 2′6′Dmt | Dap | Phe | Arg | SEQ ID No. 361 |
| 2′6′Dmt | Arg | Phe | Arg | SEQ ID No. 362 |
| 2′6′Dmt | Lys | Phe | Arg | SEQ ID No. 363 |
| 2′6′Dmt | Orn | Phe | Arg | SEQ ID No. 364 |
| 2′6′Dmt | Dab | Phe | Arg | SEQ ID No. 365 |
| 3′5′Dmt | Dap | Phe | Arg | SEQ ID No. 366 |
| 3′5′Dmt | Arg | Phe | Arg | SEQ ID No. 367 |
| 3′5′Dmt | Lys | Phe | Arg | SEQ ID No. 368 |
| 3′5′Dmt | Orn | Phe | Arg | SEQ ID No. 369 |
| 3′5′Dmt | Dab | Phe | Arg | SEQ ID No. 370 |
| Mmt | Dap | Phe | Arg | SEQ ID No. 371 |
| Mmt | Arg | Phe | Arg | SEQ ID No. 372 |
| Mmt | Lys | Phe | Arg | SEQ ID No. 373 |
| Mmt | Orn | Phe | Arg | SEQ ID No. 374 |
| Mmt | Dab | Phe | Arg | SEQ ID No. 375 |
| Hmt | Dap | Phe | Arg | SEQ ID No. 376 |
| Hmt | Arg | Phe | Arg | SEQ ID No. 377 |
| Hmt | Lys | Phe | Arg | SEQ ID No. 378 |
| Hmt | Orn | Phe | Arg | SEQ ID No. 379 |
| Hmt | Dab | Phe | Arg | SEQ ID No. 380 |
| Tmt | Dap | Phe | Arg | SEQ ID No. 381 |
| Tmt | Arg | Phe | Arg | SEQ ID No. 382 |
| Tmt | Lys | Phe | Arg | SEQ ID No. 383 |
| Tmt | Orn | Phe | Arg | SEQ ID No. 384 |
| Tmt | Dab | Phe | Arg | SEQ ID No. 385 |
| Phe | Dap | Phe | Arg | SEQ ID No. 386 |
| Phe | Arg | Phe | Arg | SEQ ID No. 387 |
| Phe | Lys | Phe | Arg | SEQ ID No. 388 |
| Phe | Orn | Phe | Arg | SEQ ID No. 389 |
| Phe | Dab | Phe | Arg | SEQ ID No. 390 |
| Tyr | Lys | Phe | Dap | SEQ ID No. 391 |
| Tyr | Lys | Phe | Arg | SEQ ID No. 392 |
| Tyr | Lys | Phe | Lys | SEQ ID No. 393 |
| Tyr | Lys | Phe | Orn | SEQ ID No. 394 |
| Tyr | Lys | Phe | Dab | SEQ ID No. 395 |
| 2′6′Dmt | Lys | Phe | Dap | SEQ ID No. 396 |
| 2′6′Dmt | Lys | Phe | Arg | SEQ ID No. 397 |
| 2′6′Dmt | Lys | Phe | Lys | SEQ ID No. 398 |
| 2′6′Dmt | Lys | Phe | Orn | SEQ ID No. 399 |
| 2′6′Dmt | Lys | Phe | Dab | SEQ ID No. 400 |
| 3′5′Dmt | Lys | Phe | Dap | SEQ ID No. 401 |
| 3′5′Dmt | Lys | Phe | Arg | SEQ ID No. 402 |
| 3′5′Dmt | Lys | Phe | Lys | SEQ ID No. 403 |
| 3′5′Dmt | Lys | Phe | Orn | SEQ ID No. 404 |
| 3′5′Dmt | Lys | Phe | Dab | SEQ ID No. 405 |
| Mmt | Lys | Phe | Dap | SEQ ID No. 406 |
| Mmt | Lys | Phe | Arg | SEQ ID No. 407 |
| Mmt | Lys | Phe | Lys | SEQ ID No. 408 |
| Mmt | Lys | Phe | Orn | SEQ ID No. 409 |
| Mmt | Lys | Phe | Dab | SEQ ID No. 410 |
| Hmt | Lys | Phe | Dap | SEQ ID No. 411 |
| Hmt | Lys | Phe | Arg | SEQ ID No. 412 |
| Hmt | Lys | Phe | Lys | SEQ ID No. 413 |
| Hmt | Lys | Phe | Orn | SEQ ID No. 414 |
| Hmt | Lys | Phe | Dab | SEQ ID No. 415 |
| Tmt | Lys | Phe | Dap | SEQ ID No. 416 |
| Tmt | Lys | Phe | Arg | SEQ ID No. 417 |
| Tmt | Lys | Phe | Lys | SEQ ID No. 418 |
| Tmt | Lys | Phe | Orn | SEQ ID No. 419 |
| Tmt | Lys | Phe | Dab | SEQ ID No. 420 |
| Phe | Lys | Phe | Dap | SEQ ID No. 421 |
| Phe | Lys | Phe | Arg | SEQ ID No. 422 |
| Phe | Lys | Phe | Lys | SEQ ID No. 423 |
| Phe | Lys | Phe | Orn | SEQ ID No. 424 |
| Phe | Lys | Phe | Dab | SEQ ID No. 425 |
| Tyr | Arg | Phe | Dap | SEQ ID No. 426 |
| Tyr | Arg | Phe | Arg | SEQ ID No. 427 |
| Tyr | Arg | Phe | Lys | SEQ ID No. 428 |
| Tyr | Arg | Phe | Orn | SEQ ID No. 429 |
| Tyr | Arg | Phe | Dab | SEQ ID No. 430 |
| 2′6′Dmt | Arg | Phe | Dap | SEQ ID No. 431 |
| 2′6′Dmt | Arg | Phe | Arg | SEQ ID No. 432 |
| 2′6′Dmt | Arg | Phe | Lys | SEQ ID No. 433 |
| 2′6′Dmt | Arg | Phe | Orn | SEQ ID No. 434 |
| 2′6′Dmt | Arg | Phe | Dab | SEQ ID No. 435 |
| 3′5′Dmt | Arg | Phe | Dap | SEQ ID No. 436 |
| 3′5′Dmt | Arg | Phe | Arg | SEQ ID No. 437 |
| 3′5′Dmt | Arg | Phe | Lys | SEQ ID No. 438 |
| 3′5′Dmt | Arg | Phe | Orn | SEQ ID No. 439 |
| 3′5′Dmt | Arg | Phe | Dab | SEQ ID No. 440 |
| Mmt | Arg | Phe | Dap | SEQ ID No. 441 |
| Mmt | Arg | Phe | Arg | SEQ ID No. 442 |
| Mmt | Arg | Phe | Lys | SEQ ID No. 443 |
| Mmt | Arg | Phe | Orn | SEQ ID No. 444 |
| Mmt | Arg | Phe | Dab | SEQ ID No. 445 |
| Hmt | Arg | Phe | Dap | SEQ ID No. 446 |
| Hmt | Arg | Phe | Arg | SEQ ID No. 447 |
| Hmt | Arg | Phe | Lys | SEQ ID No. 448 |
| Hmt | Arg | Phe | Orn | SEQ ID No. 449 |
| Hmt | Arg | Phe | Dab | SEQ ID No. 450 |
| Tmt | Arg | Phe | Dap | SEQ ID No. 451 |
| Tmt | Arg | Phe | Arg | SEQ ID No. 452 |
| Tmt | Arg | Phe | Lys | SEQ ID No. 453 |
| Tmt | Arg | Phe | Orn | SEQ ID No. 454 |
| Tmt | Arg | Phe | Dab | SEQ ID No. 455 |
| Phe | Arg | Phe | Dap | SEQ ID No. 456 |
| Phe | Arg | Phe | Arg | SEQ ID No. 457 |
| Phe | Arg | Phe | Lys | SEQ ID No. 458 |
| Phe | Arg | Phe | Orn | SEQ ID No. 459 |
| Phe | Arg | Phe | Dab | SEQ ID No. 460 |
| Lys | Tyr | Dap | Phe | SEQ ID No. 461 |
| Lys | Tyr | Arg | Phe | SEQ ID No. 462 |
| Lys | Tyr | Lys | Phe | SEQ ID No. 463 |
| Lys | Tyr | Orn | Phe | SEQ ID No. 464 |
| Lys | Tyr | Dab | Phe | SEQ ID No. 465 |
| Lys | 2′6′Dmt | Dap | Phe | SEQ ID No. 466 |
| Lys | 2′6′Dmt | Arg | Phe | SEQ ID No. 467 |
| Lys | 2′6′Dmt | Lys | Phe | SEQ ID No. 468 |
| Lys | 2′6′Dmt | Orn | Phe | SEQ ID No. 469 |
| Lys | 2′6′Dmt | Dab | Phe | SEQ ID No. 470 |
| Lys | 3′5′Dmt | Dap | Phe | SEQ ID No. 471 |
| Lys | 3′5′Dmt | Arg | Phe | SEQ ID No. 472 |
| Lys | 3′5′Dmt | Lys | Phe | SEQ ID No. 473 |
| Lys | 3′5′Dmt | Orn | Phe | SEQ ID No. 474 |
| Lys | 3′5′Dmt | Dab | Phe | SEQ ID No. 475 |
| Lys | Mmt | Dap | Phe | SEQ ID No. 476 |
| Lys | Mmt | Arg | Phe | SEQ ID No. 477 |
| Lys | Mmt | Lys | Phe | SEQ ID No. 478 |
| Lys | Mmt | Orn | Phe | SEQ ID No. 479 |
| Lys | Mmt | Dab | Phe | SEQ ID No. 480 |
| Lys | Hmt | Dap | Phe | SEQ ID No. 481 |
| Lys | Hmt | Arg | Phe | SEQ ID No. 482 |
| Lys | Hmt | Lys | Phe | SEQ ID No. 483 |
| Lys | Hmt | Orn | Phe | SEQ ID No. 484 |
| Lys | Hmt | Dab | Phe | SEQ ID No. 485 |
| Lys | Tmt | Dap | Phe | SEQ ID No. 486 |
| Lys | Tmt | Arg | Phe | SEQ ID No. 487 |
| Lys | Tmt | Lys | Phe | SEQ ID No. 488 |
| Lys | Tmt | Orn | Phe | SEQ ID No. 489 |
| Lys | Tmt | Dab | Phe | SEQ ID No. 490 |
| Lys | Phe | Dap | Phe | SEQ ID No. 491 |
| Lys | Phe | Arg | Phe | SEQ ID No. 492 |
| Lys | Phe | Lys | Phe | SEQ ID No. 493 |
| Lys | Phe | Orn | Phe | SEQ ID No. 494 |
| Lys | Phe | Dab | Phe | SEQ ID No. 495 |
| Arg | Tyr | Dap | Phe | SEQ ID No. 496 |
| Arg | Tyr | Arg | Phe | SEQ ID No. 497 |
| Arg | Tyr | Lys | Phe | SEQ ID No. 498 |
| Arg | Tyr | Orn | Phe | SEQ ID No. 499 |
| Arg | Tyr | Dab | Phe | SEQ ID No. 500 |
| Arg | 2′6′Dmt | Dap | Phe | SEQ ID No. 501 |
| Arg | 2′6′Dmt | Arg | Phe | SEQ ID No. 502 |
| Arg | 2′6′Dmt | Lys | Phe | SEQ ID No. 503 |
| Arg | 2′6′Dmt | Orn | Phe | SEQ ID No. 504 |
| Arg | 2′6′Dmt | Dab | Phe | SEQ ID No. 505 |
| Arg | 3′5′Dmt | Dap | Phe | SEQ ID No. 506 |
| Arg | 3′5′Dmt | Arg | Phe | SEQ ID No. 507 |
| Arg | 3′5′Dmt | Lys | Phe | SEQ ID No. 508 |
| Arg | 3′5′Dmt | Orn | Phe | SEQ ID No. 509 |
| Arg | 3′5′Dmt | Dab | Phe | SEQ ID No. 510 |
| Arg | Mmt | Dap | Phe | SEQ ID No. 511 |
| Arg | Mmt | Arg | Phe | SEQ ID No. 512 |
| Arg | Mmt | Lys | Phe | SEQ ID No. 513 |
| Arg | Mmt | Orn | Phe | SEQ ID No. 514 |
| Arg | Mmt | Dab | Phe | SEQ ID No. 515 |
| Arg | Hmt | Dap | Phe | SEQ ID No. 516 |
| Arg | Hmt | Arg | Phe | SEQ ID No. 517 |
| Arg | Hmt | Lys | Phe | SEQ ID No. 518 |
| Arg | Hmt | Orn | Phe | SEQ ID No. 519 |
| Arg | Hmt | Dab | Phe | SEQ ID No. 520 |
| Arg | Tmt | Dap | Phe | SEQ ID No. 521 |
| Arg | Tmt | Arg | Phe | SEQ ID No. 522 |
| Arg | Tmt | Lys | Phe | SEQ ID No. 523 |
| Arg | Tmt | Orn | Phe | SEQ ID No. 524 |
| Arg | Tmt | Dab | Phe | SEQ ID No. 525 |
| Arg | Phe | Dap | Phe | SEQ ID No. 526 |
| Arg | Phe | Arg | Phe | SEQ ID No. 527 |
| Arg | Phe | Lys | Phe | SEQ ID No. 528 |
| Arg | Phe | Orn | Phe | SEQ ID No. 529 |
| Arg | Phe | Dab | Phe | SEQ ID No. 530 |
| Dap | Tyr | D-Lys | Phe | SEQ ID No. 531 |
| Arg | Tyr | D-Lys | Phe | SEQ ID No. 532 |
| Lys | Tyr | D-Lys | Phe | SEQ ID No. 533 |
| Orn | Tyr | D-Lys | Phe | SEQ ID No. 534 |
| Dab | Tyr | D-Lys | Phe | SEQ ID No. 535 |
| Dap | 2′6′Dmt | D-Lys | Phe | SEQ ID No. 536 |
| Arg | 2′6′Dmt | D-Lys | Phe | SEQ ID No. 537 |
| Lys | 2′6′Dmt | D-Lys | Phe | SEQ ID No. 538 |
| Orn | 2′6′Dmt | D-Lys | Phe | SEQ ID No. 539 |
| Dab | 2′6′Dmt | D-Lys | Phe | SEQ ID No. 540 |
| Dap | 3′5′Dmt | D-Lys | Phe | SEQ ID No. 541 |
| Arg | 3′5′Dmt | D-Lys | Phe | SEQ ID No. 542 |
| Lys | 3′5′Dmt | D-Lys | Phe | SEQ ID No. 543 |
| Orn | 3′5′Dmt | D-Lys | Phe | SEQ ID No. 544 |
| Dab | 3′5′Dmt | D-Lys | Phe | SEQ ID No. 545 |
| Dap | Mmt | D-Lys | Phe | SEQ ID No. 546 |
| Arg | Mmt | D-Lys | Phe | SEQ ID No. 547 |
| Lys | Mmt | D-Lys | Phe | SEQ ID No. 548 |
| Orn | Mmt | D-Lys | Phe | SEQ ID No. 549 |
| Dab | Mmt | D-Lys | Phe | SEQ ID No. 550 |
| Dap | Hmt | D-Lys | Phe | SEQ ID No. 551 |
| Arg | Hmt | D-Lys | Phe | SEQ ID No. 552 |
| Lys | Hmt | D-Lys | Phe | SEQ ID No. 553 |
| Orn | Hmt | D-Lys | Phe | SEQ ID No. 554 |
| Dab | Hmt | D-Lys | Phe | SEQ ID No. 555 |
| Dap | Tmt | D-Lys | Phe | SEQ ID No. 556 |
| Arg | Tmt | D-Lys | Phe | SEQ ID No. 557 |
| Lys | Tmt | D-Lys | Phe | SEQ ID No. 558 |
| Orn | Tmt | D-Lys | Phe | SEQ ID No. 559 |
| Dab | Tmt | D-Lys | Phe | SEQ ID No. 560 |
| Dap | Phe | D-Lys | Phe | SEQ ID No. 561 |
| Arg | Phe | D-Lys | Phe | SEQ ID No. 562 |
| Lys | Phe | D-Lys | Phe | SEQ ID No. 563 |
| Orn | Phe | D-Lys | Phe | SEQ ID No. 564 |
| Dal | Phe | D-Lys | Phe | SEQ ID No. 565 |
| Dap | Tyr | Lys | Phe | SEQ ID No. 566 |
| Arg | Tyr | Lys | Phe | SEQ ID No. 567 |
| Lys | Tyr | Lys | Phe | SEQ ID No. 568 |
| Orn | Tyr | Lys | Phe | SEQ ID No. 569 |
| Dab | Tyr | Lys | Phe | SEQ ID No. 570 |
| Dap | 2′6′Dmt | Lys | Phe | SEQ ID No. 571 |
| Arg | 2′6′Dmt | Lys | Phe | SEQ ID No. 572 |
| Lys | 2′6′Dmt | Lys | Phe | SEQ ID No. 573 |
| Orn | 2′6′Dmt | Lys | Phe | SEQ ID No. 574 |
| Dab | 2′6′Dmt | Lys | Phe | SEQ ID No. 575 |
| Dap | 3′5′Dmt | Lys | Phe | SEQ ID No. 576 |
| Arg | 3′5′Dmt | Lys | Phe | SEQ ID No. 577 |
| Lys | 3′5′Dmt | Lys | Phe | SEQ ID No. 578 |
| Orn | 3′5′Dmt | Lys | Phe | SEQ ID No. 579 |
| Dab | 3′5′Dmt | Lys | Phe | SEQ ID No. 580 |
| Dap | Mmt | Lys | Phe | SEQ ID No. 581 |
| Arg | Mmt | Lys | Phe | SEQ ID No. 582 |
| Lys | Mmt | Lys | Phe | SEQ ID No. 583 |
| Orn | Mmt | Lys | Phe | SEQ ID No. 584 |
| Dab | Mmt | Lys | Phe | SEQ ID No. 585 |
| Dap | Hmt | Lys | Phe | SEQ ID No. 586 |
| Arg | Hmt | Lys | Phe | SEQ ID No. 587 |
| Lys | Hmt | Lys | Phe | SEQ ID No. 588 |
| Orn | Hmt | Lys | Phe | SEQ ID No. 589 |
| Dab | Hmt | Lys | Phe | SEQ ID No. 590 |
| Dap | Tmt | Lys | Phe | SEQ ID No. 591 |
| Arg | Tmt | Lys | Phe | SEQ ID No. 592 |
| Lys | Tmt | Lys | Phe | SEQ ID No. 593 |
| Orn | Tmt | Lys | Phe | SEQ ID No. 594 |
| Dab | Tmt | Lys | Phe | SEQ ID No. 595 |
| Dap | Phe | Lys | Phe | SEQ ID No. 596 |
| Arg | Phe | Lys | Phe | SEQ ID No. 597 |
| Lys | Phe | Lys | Phe | SEQ ID No. 598 |
| Orn | Phe | Lys | Phe | SEQ ID No. 599 |
| Dab | Phe | Lys | Phe | SEQ ID No. 600 |
| Dap | Tyr | Arg | Phe | SEQ ID No. 601 |
| Arg | Tyr | Arg | Phe | SEQ ID No. 602 |
| Lys | Tyr | Arg | Phe | SEQ ID No. 603 |
| Orn | Tyr | Arg | Phe | SEQ ID No. 604 |
| Dab | Tyr | Arg | Phe | SEQ ID No. 605 |
| Dap | 2′6′Dmt | Arg | Phe | SEQ ID No. 606 |
| Arg | 2′6′Dmt | Arg | Phe | SEQ ID No. 607 |
| Lys | 2′6′Dmt | Arg | Phe | SEQ ID No. 608 |
| Orn | 2′6′Dmt | Arg | Phe | SEQ ID No. 609 |
| Dab | 2′6′Dmt | Arg | Phe | SEQ ID No. 610 |
| Dap | 3′5′Dmt | Arg | Phe | SEQ ID No. 611 |
| Arg | 3′5′Dmt | Arg | Phe | SEQ ID No. 612 |
| Lys | 3′5′Dmt | Arg | Phe | SEQ ID No. 613 |
| Orn | 3′5′Dmt | Arg | Phe | SEQ ID No. 614 |
| Dab | 3′5′Dmt | Arg | Phe | SEQ ID No. 615 |
| Dap | Mmt | Arg | Phe | SEQ ID No. 616 |
| Arg | Mmt | Arg | Phe | SEQ ID No. 617 |
| Lys | Mmt | Arg | Phe | SEQ ID No. 618 |
| Orn | Mmt | Arg | Phe | SEQ ID No. 619 |
| Dab | Mmt | Arg | Phe | SEQ ID No. 620 |
| Dap | Hmt | Arg | Phe | SEQ ID No. 621 |
| Arg | Hmt | Arg | Phe | SEQ ID No. 622 |
| Lys | Hmt | Arg | Phe | SEQ ID No. 623 |
| Orn | Hmt | Arg | Phe | SEQ ID No. 624 |
| Dab | Hmt | Arg | Phe | SEQ ID No. 625 |
| Dap | Tmt | Arg | Phe | SEQ ID No. 626 |
| Arg | Tmt | Arg | Phe | SEQ ID No. 627 |
| Lys | Tmt | Arg | Phe | SEQ ID No. 628 |
| Orn | Tmt | Arg | Phe | SEQ ID No. 629 |
| Dab | Tmt | Arg | Phe | SEQ ID No. 630 |
| Dap | Phe | Arg | Phe | SEQ ID No. 631 |
| Arg | Phe | Arg | Phe | SEQ ID No. 632 |
| Lys | Phe | Arg | Phe | SEQ ID No. 633 |
| Orn | Phe | Arg | Phe | SEQ ID No. 634 |
| Dab | Phe | Arg | Phe | SEQ ID No. 635 |

Known bioactive mitochondria targeting peptides including SS-01 (Tyr-D-Arg-Phe-Lys), SS-02 (2,6, Dmt-D-Arg-Phe-Lys), SS-20 (Phe-D-Arg-Phe-Lys), elamipretide (H-d-Arg-Dmt-Lys-Phe-NH2) and EY005 (H-d-Arg-Dmt-Lys-Phe(-OH) share common traits including being tetramers and containing alternating cationic and aromatic amino acids. Table 1 includes potential peptide analogs which share these properties, and which could be synthesized as prodrugs for use in Mito XR. Notably functional analogs of known active mitochondria targeting peptides can be designed by conservative substitution of one amino acid for another with similar biophysical properties. Thus, functional analogs of peptides found in Table 1 (and SEQ ID NOs. 1-635) could be created by conservative substitution of other natural, synthetic or unnatural amino acids with similar properties (e.g., by substitution of aromatic amino acids for other aromatic amino acids, or cationic amino acids for other cationic amino acids). Naturally occurring cationic amino acids include lysine, arginine and histidine and other cationic amino acids include but are not limited to diaminobutyric acid (Dab), diaminopropionic acid (Dap) and ornithine. Naturally occurring aromatic amino acids include phenylalanine, tyrosine, tryptophan, histidine and other aromatic amino acids include but are not limited to Dmt=dimethyltyrosine (Dmt), 2′-methyltyrosine (Mmt), N,2′,6′-trimethyltyrosine (TmT) and 2′-hydroxy, 6′-methyltyrosine (Hmt). Additionally, and in some cases preferably, D-amino acids can be substituted for L-amino acids which may render mitochondria targeting peptides resistant to degradation by protease and peptidase enzymes.

In general, these mitochondrial targeted drugs are imported into mitochondria due to the positive charge of the tetrapeptide motif. All of these mitochondrial targeted drugs facilitate restoration of normal mitochondrial function by binding of the aromatic residues to peroxidized cardiolipin in the cristae, restoring cristae morphology and electron transport chain (ETC) function, as described in FIGS. 1A-1C. These effects have been observed with elamipretide and EY005 with comparable potency, which is not unexpected since both drugs share comparable physicochemical, biochemical, and pharmacologic properties.

The covalently linked conjugation moieties of MTT-prodrugs form noncovalent avid interactions (or binding) to one of six different classes of substances formulated as irregularly shaped particulates: fatty acid, organic molecules that can form keto-enol tautomers, charged phospholipid, charged protein, nucleic acid, and polysaccharides. The formation of MTT-prodrug-complex particulates optimizes the drug's physicochemical properties for compatibility with the complexation-based extended release drug delivery system (XRDDS) that is formed by admixture of one or more MTT-prodrug-complex particulates in a hydrophobic dispersal medium, enabling controlled, extended release from the stable multiphasic colloidal suspension that is specifically formulated for intravitreal (IVT) or periocular administration.

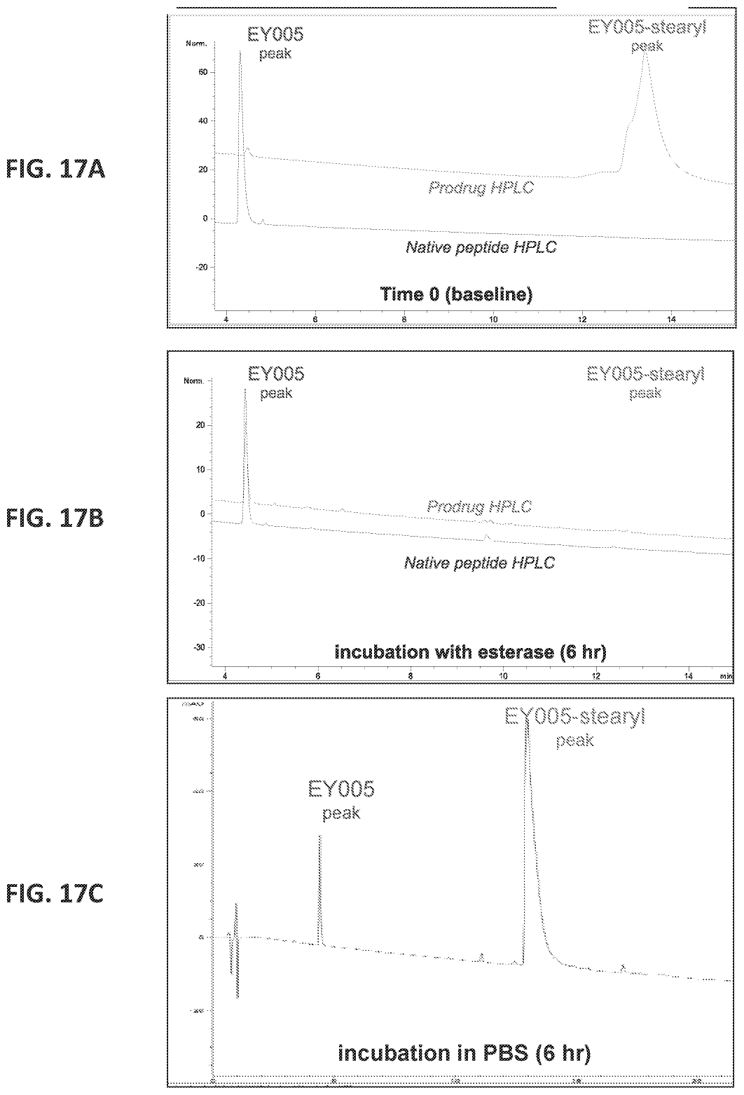

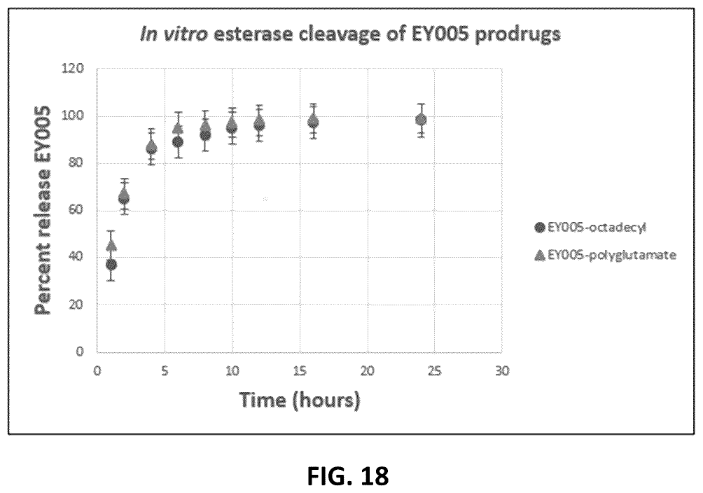

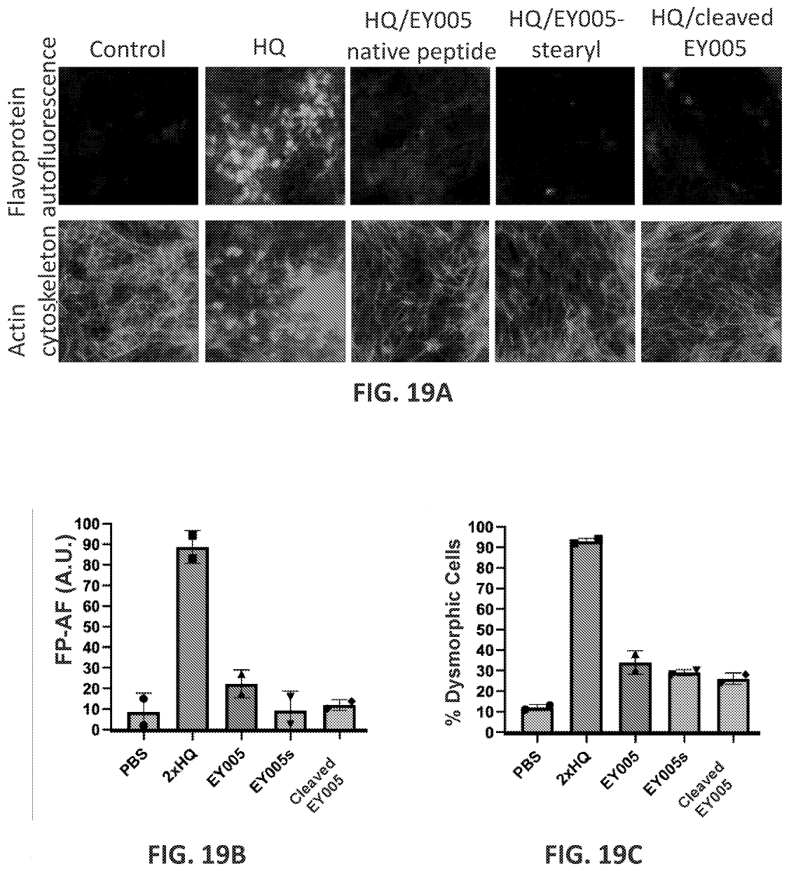

As mentioned above, one feature of the MTT-prodrug is that the bond linking bioactive MTT to the inactive conjugation moiety is readily cleaved by enzymatic reaction, catalysis, hydrolysis, or other chemical reaction (FIGS. 17A-17C, 18). Upon cleavage of this bond in the MTT-prodrug, the released MTT retains full bioactivity for prevention or reversal of mitochondrial dysfunction (FIGS. 19A-19C).

Numerous metabolizing enzymes have been detected in ocular tissues, including esterases, peptidases, phosphatases, oxime hydrolases, ketone reductases, and others. The linkage to the conjugation moiety for any of the mitochondrial targeted tetrapeptides described herein may be configured to achieve specific cleavage by any of these metabolizing enzymes.

In some examples, prodrugs of mitochondria targeting peptides may be formed by linking various conjugation moieties to the mitochondria targeting peptide via any of several types of dynamic covalent bonds which include but are not limited to ester bonds, hydrazone/imine bonds, disulfide bonds, thioester bonds, thioether bonds, phosphonate ester bonds, boronate ester bonds, amide bonds, carbamate ester bonds, carbonate ester bonds or others known to those practiced in the art of medicinal chemistry.

Ester prodrugs in particular may be desirable for IVT drug formulations since the vitreous and retina contain abundant esterase activity.

For example, from among the class of MTTs, one particular MTT, H-d-Arg-DMT-Lys-Phe, referred to herein as EY005 (FIG. 4A):

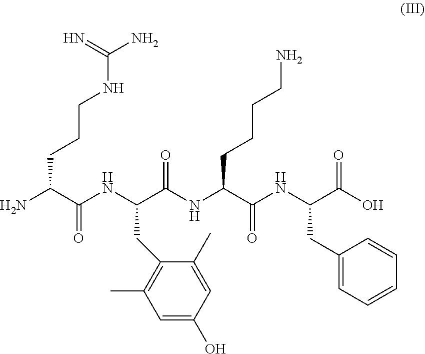 [SMILES: Cc1cc(O)cc(C)c1C[C@H](NC(=O)[C@H](N)CCCNC(=N)N)C(=O)N[C@@H](CCCCN)C(=O)N[C@@H](Cc1ccccc1)C(=O)O]

EY005 may be formed into a prodrug by condensation reaction or esterification to a conjugation group R, where R is a specific conjugation moiety, linked via a carboxyl ester, a phosphate ester, or a carbamate ester, to form H-d-Arg-DMT-Lys-Phe(-O)—R (FIG. 4B):

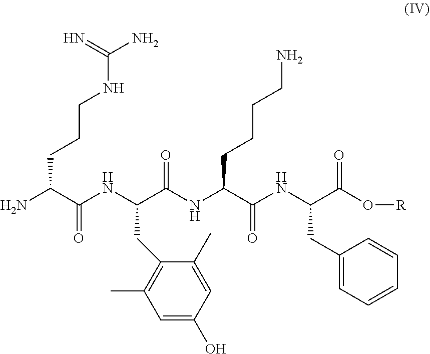

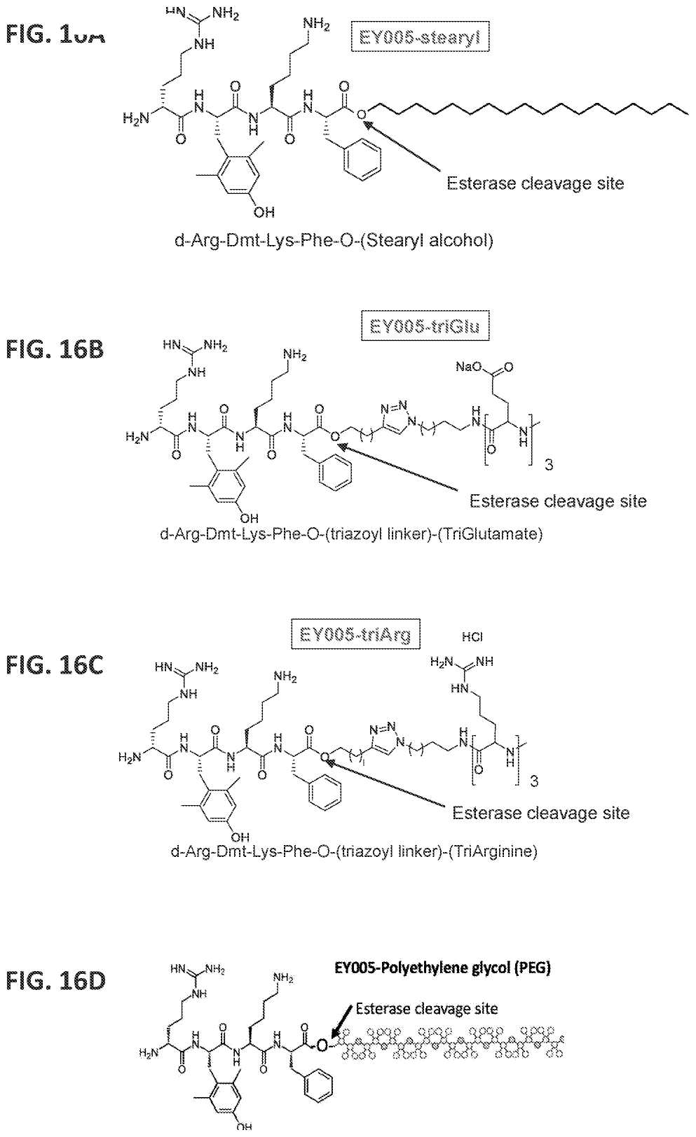

In the case of EY005 and other MTTs, R is covalently linked via ester bond at the hydroxyl group of the amino acid in the 4th position of the MTT and is selected from among one of the following five classes of chemical substances: a C4-C30 lipid moiety, an C4-C30 straight-chain or branched aliphatic moiety, a 2-mer to 30-mer peptide moiety, a pegylated moiety, or a carbohydrate moiety. This structure is also shown in FIG. 4B. FIGS. 16A-16D illustrate examples of prodrugs of EY005 (H-d-Arg-DMT-Lys-Phe(-OH)).

In some examples, cleavage and release of the free bioactive mitochondrial targeted tetrapeptide can be assessed in an in vitro release assay, wherein the ester-based prodrug is incubated in a solution containing carboxyesterase (or other natural or synthetic esterase), isolated vitreous recovered from animal (e.g., pig, rabbit, etc.), or isolated vitreous recovered from human donor, at 37 degrees Celsius, 25 degrees Celsius, or other temperatures. Analytic methods such as HPLC or mass spectrometry can be used to calculate the amount of free bioactive mitochondrial targeted tetrapeptide and intact prodrug, at various timepoints after start of incubation (FIGS. 17A-17B, FIG. 18).

In some examples, cleavage and release of the free bioactive mitochondrial targeted tetrapeptide can be assessed in an in vitro release assay, wherein the ester-based prodrug is incubated in media, at 37 degrees Celsius, 25 degrees Celsius, or other temperatures. Analytic methods such as HPLC or mass spectrometry can be used to calculate the amount of free bioactive mitochondrial targeted tetrapeptide and intact prodrug, at various timepoints after start of incubation (FIG. 17C).

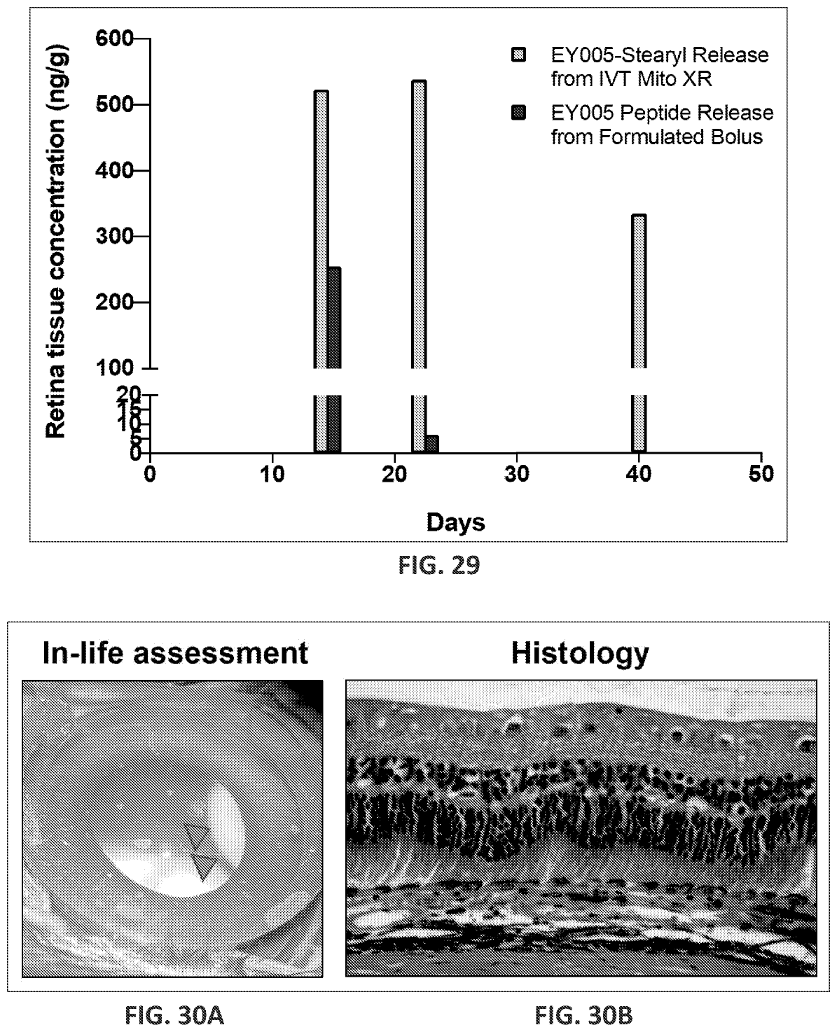

In some examples, cleavage and release of the free bioactive mitochondrial targeted tetrapeptide can be assessed following in vivo injection of the prodrug into the vitreous cavity or periocular tissues of a preclinical animal model (e.g., mouse, rat, rabbit, pig, etc.) (FIGS. 29, 31, 32), wherein ocular tissue is recovered, and analytic methods such as HPLC or mass spectrometry can be used to calculate the amount of free bioactive mitochondrial targeted tetrapeptide and intact prodrug, at various timepoints after in vivo injection.

One specific example of EY005-prodrug includes EY005-stearyl (depicted in FIG. 16A), wherein EY005 is linked via ester bond to stearyl alcohol, one member from the group of long-chain saturated fatty alcohols. On cleavage of the ester bond, the prodrug EY005-stearyl releases the EY005 MTT. To demonstrate this experimentally, EY005-stearyl was incubated at 37° C. in vitro with carboxyesterase (0.1 μg/mL), to simulate the ocular physiologic environment and the type of esterase that is readily abundant therein, within the vitreous. Incubation of EY005-stearyl with carboxyesterase produced rapid cleavage of the prodrug ester bond, releasing EY005, as evident by high performance liquid chromatography (HPLC) analysis and quantification of EY005 MTT and EY005-stearyl prodrug in solution (FIGS. 17A-17B). Upon addition of EY005-stearyl prodrug to phosphate-buffered saline solution at 37° C. without esterase, the ester bond of the EY005-stearyl prodrug cleaves more slowly (˜36 hours) by hydrolysis (FIG. 17C). Thus, in ocular physiologic system, the covalent bond of the prodrug linking MTT to inactive conjugation is readily cleaved either by enzymatic cleavage or more slowly by hydrolysis, releasing the active MTT. Thus, in ocular physiologic system, the covalent bond of the prodrug linking MTT to inactive conjugation is readily cleaved either by enzymatic cleavage or more slowly by hydrolysis, releasing the active MTT.

Mitochondrial dysfunction can be modeled using in vitro cell culture systems relevant to retinal disease. In one such system, retinal pigment epithelium (RPE) cells are cultured to post-confluence to establish a differentiated cell monolayer and are then assayed for mitochondrial dysfunction and effect of drugs for treatment of mitochondrial dysfunction (FIG. 5). In this model, mitochondrial dysfunction is induced by two exposures to nonlethal doses of hydroquinone (HQ) at day −3 and day 0. Hydroquinone (HQ), a well-known environmental toxicant ubiquitously present in Western lifestyle, is a biochemical inducer of mitochondrial dysfunction. When HQ is added to fully differentiated RPE cell cultures, HQ competes with mitochondrial ubiquinone, siphoning high-energy electrons from electron transport chain, which then reduces oxygen into superoxide, leading to cardiolipin peroxidation and mitochondrial dysfunction. Cells are then treated with a drug of interest (mitochondrial targeted peptide drug or a prodrug thereof) or control (phosphate-buffered saline (PBS) solution and assayed for effects on mitochondrial dysfunction, as measured by microscopy of flavoprotein autofluorescence (a measure of mitochondrial electron transport chain complex II decompensation) (FIG. 6).

A rabbit model of mitochondrial dysfunction is also used to assay bioactivity of MTT and prodrugs derived thereof. For example, FIG. 12 shows a protocol that was used; at days 0 and 1, rabbits receive IVT HQ (0.05 mL, 250 mM). On day 2, the MTT (e.g., EY005, 15 μM) were injected IVT. On day 3, a fluorogenic dye that measures hydroxyl, peroxyl and other reactive oxygen species (e.g., DCFDA) was injected IVT, and tissues were collected 12 hours later. RPE flatmounts were processed for multiple readouts: oxidation byproducts (DCFDA, FIG. 13) and measures of RPE-associated extracellular matrix (cytosolic vimentin expression, FIG. 14), and RPE cell dysmorphology (i.e., actin cytoskeleton (phalloidin), FIG. 14). All were analyzed and quantified by microscopy.

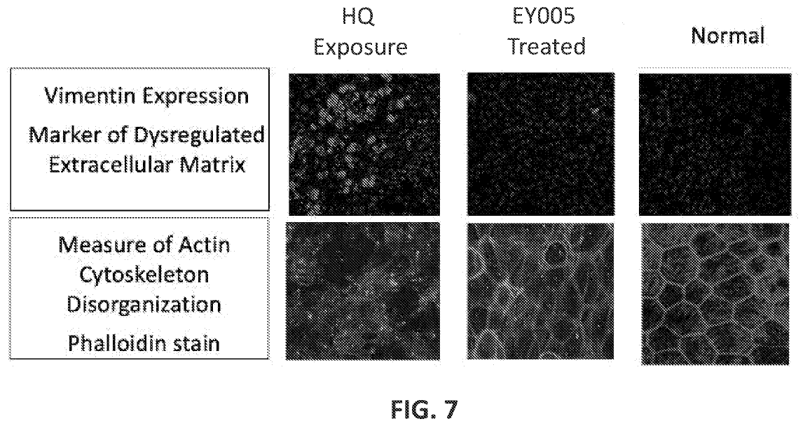

In addition, unexpected and nonobvious effects of treatment with high dose (>5 μM) mitochondrial targeted tetrapeptide (e.g., EY005), include reversal of dysregulated extracellular matrix in cultured RPE cells or in rabbit model of dry AMD as reflected by reduced vimentin expression (FIGS. 7, 9, 14) and reversal of actin cytoskeleton disorganization as reflected by phalloidin staining (see FIGS. 7, 10, 14).

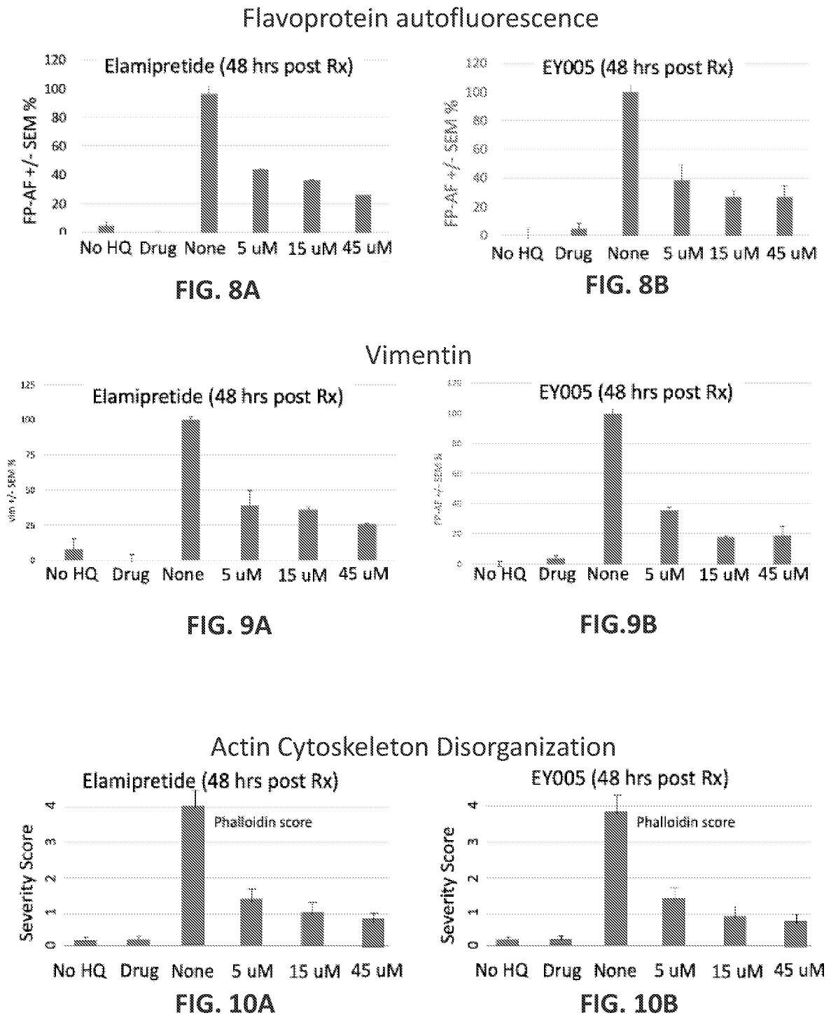

In a particular example, treatment of HQ-exposed RPE cells with MTTs elamipretide and EY005 (H-d-Arg-DMT-Lys-Phe) (FIGS. 8A-8B, 9A-9B, and 10A-10B) produce near complete reversal of mitochondrial dysfunction, substantially reducing flavoprotein autofluorescence as compared to control HQ-exposed cells (FIGS. 8A-8B). EY005 treatment reversed mitochondrial dysfunction (FIG. 8A), substantially reducing flavoprotein autofluorescence as compared to control (“None” or “no drug”). In assessing dose-response efficacy, EY005 was highly potent, with comparable or equivalent dose-response and potency to elamipretide (FIG. 8B) in the same assay.

Similarly, EY005 (15 μM) was potent for reversal of mitochondrial dysfunction in the rabbit ocular hydroquinone model, as well (FIG. 13).

Upon cleavage of the covalent bond of the MTT-prodrug, the native MTT peptide retains bioactivity for treatment of mitochondrial dysfunction. For example, as depicted in FIGS. 19A-19C, in an in vitro cell culture model of dry AMD, EY005-stearyl (5 μM) was added to RPE cells (which possess endogenous esterases) with mitochondrial dysfunction induced by exposure to hydroquinone (HQ). EY005-stearyl effectively reversed HQ-induced mitochondrial dysfunction in RPE cells (as depicted by cellular flavoprotein-autofluorescence), with efficacy equivalent to treatment with EY005 native peptide (5 μM). EY005-stearyl was also preincubated with carboxyesterase (0.1 μg/mL) in separate media. Recovered media containing cleaved EY005 (5 μM) was added to this RPE cellular model of mitochondrial dysfunction, and this was similarly effective and equipotent to EY005 native peptide for the reversal of RPE mitochondrial dysfunction. Thus, these studies affirm that the active MTT that is cleaved from the MTT-prodrug retains essential and unmodified bioactivity for the treatment of mitochondrial dysfunction.

Any of the class of MTT described herein, R′, including the example MTT H-d-Arg-DMT-Lys-Phe (—OH) (“EY005”), or may be covalently linked to a variety of conjugation moieties.

In general, the conjugation moiety, R, to which the MTT is covalently linked, is not selected on the basis of bioactivity for prevention or reversal of mitochondrial dysfunction.

Also disclosed herein are MTT-prodrugs comprising homo- or hetero-dimers, trimers, multimers of any MTT, either linked together directly as a polypeptide or indirectly to a chemical substance that serves a linker moiety, which could functionally serve as a cleavable conjugation moiety

As described herein, MTT, R′, may be covalently linked to conjugation moiety R, selected from among one of the following five classes of chemical substances: a C4-C30 lipid moiety, a C4-C30 straight-chain or branched aliphatic moiety, a 2-mer to 30-mer peptide moiety, a pegylated moiety, or a carbohydrate moiety.

One class of conjugation moieties is C4-C30 lipid moiety, with or without a preceding linker moiety that bonds the lipid moiety to the fourth amino acid of the MTT. Herein, lipid is defined as organic compounds that are insoluble in water but soluble in organic solvents. Lipids include fatty acids, fatty alcohols, glycerolipids, glycerophospholipids, sphingolipids, saccharolipids, polyketides (derived from condensation of ketoacyl subunits), sterol lipids, prenol lipids (derived from condensation of isoprene subunits), phospholipids, oils, waxes, and steroids.

One class of conjugation moieties is C4-C30 straight-chain or branched aliphatic moiety, with or without a preceding linker moiety that bonds the aliphatic hydrocarbon, to the fourth amino acid of the MTT. This class include alkanes, alkenes, and alkynes and other hydrocarbon moieties made up of 4 to about 30 carbons.

One class of conjugation moieties is peptide moiety, with or without a preceding linker moiety that bonds the peptide to the fourth amino acid of the MTT, wherein the peptide moiety comprises a natural or synthetic amino acid polymer or polypeptide chain with length of 2-mer to 30-mer, which may be anionic, cationic, or neutral in charge and contain homogeneous or heterogeneous amino acid repeats.

Anionic peptide moiety may include at least one of: poly-glutamate, poly-aspartate or a combination of glutamate and aspartate.

Cationic peptide moiety may include at least one of: poly-arginine, poly-lysine, poly-histidine, a combination of arginine and lysine, a combination of arginine and histidine, a combination of histidine and lysine, a combination of arginine, histidine, and lysine.

The peptide moiety may have one or more PEGylation sites for addition of polyethylene glycol (PEG) groups or may have one or more sites for modification by addition of sugar or carbohydrate molecules, including glycosylation.

One class of conjugation moieties is pegylated compound moiety, with or without a preceding linker moiety that bonds the pegylated compound to the fourth amino acid of the MTT, including polyethylene glycol (PEG) polymers of linear, branched, Y-shaped, or multi-arm geometries, pegylated peptides or proteins, or pegylated succinates such as succinimidyl succinate.

One class of conjugation moieties is carbohydrate molecular moiety, with or without a preceding linker moiety that bonds the carbohydrate to the fourth amino acid of the MTT, including but not limited to monosaccharides or oligosaccharides of 2 to 20 sugars. The carbohydrate molecule may comprise one or more of: glucose, galactose, lactose, mannose, ribose, fucose, N-acetylgalactosamine, N-acetylglucosamine, N-acetyleneuraminic acid, or an epimer or derivative of any of these.

In some instances, a conjugation moiety, which may be combine elements from two or more of these classes, may serve as as a multimeric linker moiety that is convalently linked to multiple mitochondria targeting peptides to form dimers and/or multimers. Such linkers capable of generating dimers or multimers of mitochondria targeting peptides may be referred to as “multimerization domains.” MTT prodrug with multimerization domain has formula (V):

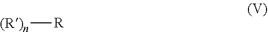

wherein R is a linker or multimerization domain which is convalently linked to multiple mitochondria targeting peptides R′, to form dimers or multimers of the prodrug and n is equal to 2 to about 100. Examples include PEG polymers, polyvinyl alcohol (PVA) polymers, or polypeptides, where the linker conjugation moiety R is covalently linked to two or more molecules of the MTT R′, to form dimers, trimers, multimers, etc. In some cases, the multimerization domains have alcohols, i.e., multiple “—OH” groups, to which the MTT units R′ are bound. In this setting, multiple MTT covalently linked (e.g., via ester or another dynamic covalent bond) to the multimerization domain may be referred to an MTT prodrug multimer.

For example, a prodrug compound may have the formula, where “n” is number comprising PVA polymer:

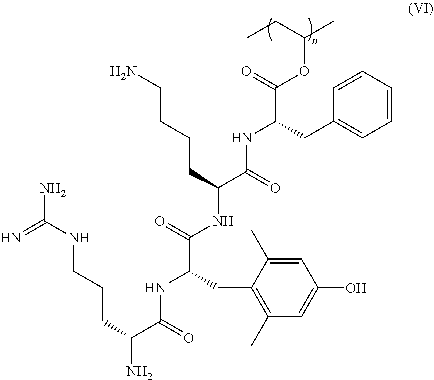

As described herein, the prodrug may be a product of a condensation or esterification reaction between mitochondrial targeted peptide and a fatty alcohol, ranging in length from C4 (four carbons) to C30 (30 carbons).

The fatty alcohol may comprise one or more of: tert-butyl alcohol, tert-amyl alcohol, 3-methyl-3-pentanol, 1-heptanol (enanthic alcohol), 1-octanol (capryl alcohol), 1-nonanol (pelargonic alcohol), 1-decanol (decyl alcohol, capric alcohol), undecyl alcohol (1-undecanol, undecanol, hendecanol), dodecanol (1-dodecanol, lauryl alcohol), tridecyl alcohol (1-tridecanol, tridecanol, isotridecanol), 1-tetradecanol (myristyl alcohol), pentadecyl alcohol (1-pentadecanol, pentadecanol), 1-hexadecanol (cetyl alcohol), cis-9-hexadecen-1-ol (palmitoleyl alcohol), heptadecyl alcohol (1-n-heptadecanol, heptadecanol), 1-octadecanol (stearyl alcohol), 1-octadecenol (oleyl alcohol), 1-nonadecanol (nonadecyl alcohol), 1-eicosanol (arachidyl alcohol), 1-heneicosanol (heneicosyl alcohol), 1-docosanol (behenyl alcohol), cis-13-docosen-1-ol (erucyl alcohol), 1-tetracosanol (lignoceryl alcohol), 1-pentacosanol, 1-hexacosanol (ceryl alcohol), 1-heptacosanol, 1-octacosanol (montanyl alcohol, cluytyl alcohol), 1-nonacosanol, 1-triacontanol (myricyl alcohol, melissyl alcohol).

The R may be a C30 alkyl (triacontanyl) group or the O—R is a C30 fatty alcohol (myricyl alcohol).

For example, the R may be a C28 alkyl (octasanyl) group or the O—R is a C28 fatty alcohol (montanyl alcohol).

The R may be a C26 alkyl (hexacosanyl) group or the O—R is a C26 fatty alcohol (ceryl alcohol).

The R may be a C24 alkyl (tetracosanyl) group or the O—R is a C24 fatty alcohol (lignoceryl alcohol).

The R may be a C22 alkyl (docosanyl) group or the O—R is a C22 fatty alcohol (behenyl alcohol).

The R may be a C20 alkyl (eicosanyl) group or the O—R is a C20 fatty alcohol (arachidyl alcohol).

The R may be a C18 alkyl (octadecyl) group or the O—R is a C18 fatty alcohol (stearyl alcohol).

The R may be a C16 alkyl (hexadecyl) group or the O—R is a C16 fatty alcohol (palmityl alcohol).

The R may be a C14 alkyl (tetradecyl) group or the O—R is a C14 fatty alcohol (myristyl alcohol).

The R may be a C12 alkyl (dodecyl) group or the O—R is a C12 fatty alcohol (lauryl alcohol).

The R may be a C10 alkyl (decyl) group or the O—R is a C10 fatty alcohol (decanol).

The prodrug may be a product of a condensation or esterification reaction between mitochondrial targeted peptide and a fatty acid, ranging in length from C4 (four carbons) to C34 (34 carbons).

The fatty acid may comprise one or more of: Tetradecanoic acid, pentadecanoic acid, (9Z)-hexadecenoic acid, Hexadecanoic acid, Heptadecanoic acid, Octadecanoic acid, (9Z,12Z)-octadeca-9,12-dienoic acid, (9Z,12Z,15Z)-octadeca-9,12,15-trienoic acid, (6Z,9Z,12Z)-octadeca-6,9,12-trienoic acid, (5E,9E,12E)-octadeca-5,9,12-trienoic acid, (6Z,9Z,12Z,15Z)-octadeca-6,9,12,15-tetraenoic acid, (Z)-octadec-9-enoic acid, (11E)-octadec-11-enoic acid, (E)-octadec-9-enoic acid, nonadecanoic acid, and eicosanoic acid.

In some examples, a member of the class of mitochondrial targeted tetrapeptides is manufactured as a peptide-based prodrug via addition of an aliphatic group having from about 4 to about 30 carbons as a conjugation moiety to the hydroxyl group from the amino acid in the 4^{th} position of the mitochondrial targeted tetrapeptide. Aliphatic groups may include alkanes, alkenes, and alkynes and include both unbranched, branched and cyclic groups.

In some examples, a member of the class of mitochondrial targeted tetrapeptides is manufactured as a peptide-based prodrug via addition of a peptide as a conjugation moiety to the hydroxyl group from the amino acid in the 4th position of the mitochondrial targeted tetrapeptide. The peptide-based prodrug includes a conjugation moiety that is a small peptide (e.g., between 2-30 amino acids (AA)).

In any of these prodrugs, R may be a 2-mer to about a 30-mer peptide moiety comprising natural or synthetic amino acids, with or without a preceding linker moiety that bonds the peptide to the hydroxyl (—OH) of fourth amino acid of the mitochondrial targeted peptide. R may be a 2-mer to about a 30-mer anionic peptide moiety, with or without a linker moiety that bonds the peptide to the hydroxyl (—OH) of the fourth amino acid of the mitochondrial targeted peptide.

Examples of anionic peptide sequences that may serve as conjugation moiety groups R include but are not limited to: poly-aspartic acid (aspartate), poly-glutamic acid (glutamate), peptides comprising poly-(aspartic acid-glutamic acid) or poly-(glutamic acid-aspartic acid) repeats.

The anionic peptide moiety may include at least one of: poly-glutamate, poly-aspartate or a combination of glutamate and aspartate.

R may be a 2-mer to about a 30-mer cationic peptide, with or without a linker moiety that bonds the peptide to the hydroxyl (—OH) of the fourth amino acid of the mitochondrial targeted peptide.

Examples of cationic peptide sequences that may serve as conjugation moiety groups R include but are not limited to: poly-lysine, poly-arginine, poly-histidine, peptides comprising poly-(lysine-arginine) (or arginine-lysine) repeats, peptides comprising poly-(lysine-histidine) (or histidine-lysine) repeats, peptides comprising poly-(arginine-histidine) (or histidine-arginine) repeats, peptides comprising poly-(lysine-arginine-histidine) repeats, peptides comprising poly-(lysine-histidine-arginine) repeats, peptides comprising poly-(arginine-lysine-histidine) repeats, peptides comprising poly-(arginine-histidine-lysine) repeats, peptides comprising poly-(histidine-arginine-lysine) repeats, peptides comprising poly-(histidine-lysine-arginine) repeats.

R may be a 2-mer to about a 30-mer peptide moiety comprising natural or synthetic amino acids, with a preceding linker moiety that bonds the peptide to the hydroxyl (—OH) of the fourth amino acid of the mitochondrial targeted peptide. The peptide moiety may have one or more PEGylation sites for addition of polyethylene glycol (PEG) groups.

R may be a 2-mer to about a 30-mer peptide moiety comprising natural or synthetic amino acids, without a preceding linker moiety that bonds the peptide to the hydroxyl (—OH) of the fourth amino acid of the mitochondrial targeted peptide. The peptide moiety may have one or more sites for modification by addition of sugar or carbohydrate molecules, including glycosylation.

R may be a 2-mer to about a 30-mer polyarginine moiety, with or without a preceding linker moiety that bonds the peptide to the hydroxyl (—OH) of the fourth amino acid of the mitochondrial targeted peptide.

In some examples the prodrug compound is H-d-Arg-DMT-Lys-Phe(-O)—R, wherein R is a 6-mer polyarginine moiety, with or without a preceding linker moiety that bonds the peptide to the hydroxyl (—OH) of the fourth amino acid of the mitochondrial targeted peptide.

In any of these prodrug compounds, R may be a 2-mer to about a 30-mer polyaspartate moiety, with or without a preceding linker moiety that bonds the peptide to the hydroxyl (—OH) the fourth amino acid of the mitochondrial targeted peptide.

R may be a 2-mer to about a 30-mer polyhistidine moiety, with or without a preceding linker moiety that bonds the peptide to the hydroxyl (—OH) of the fourth amino acid of the mitochondrial targeted peptide.

R may be a 2-mer to about a 30-mer polylysine moiety, with or without a preceding linker moiety that bonds the peptide to the hydroxyl (—OH) of the fourth amino acid of the mitochondrial targeted peptide.

R may be a polyethylene glycol (PEG) polymer, a pegylated peptide, or pegylated succinate including PEG polymers of linear, branched, Y-shaped, or multi-arm geometries.

R may be a carbohydrate moiety comprising a carbohydrate molecule comprising a monosaccharide or oligosaccharide of 2 to 20 sugars which is covalently bound to the hydroxyl (—OH) of the fourth amino acid of the mitochondrial targeted peptide. For example, the carbohydrate molecule may comprise one of a: glucose, galactose, lactose, mannose, ribose, fucose, N-acetylgalactosamine, N-acetylglucosamine, N-acetyleneuraminic acid, or an epimer or derivative of glucose, galactose, lactose, mannose, ribose, fucose, N-acetylgalactosamine, N-acetylglucosamine, and N-acetyleneuraminic acid.

R may be a 2-mer to about a 30-mer polyglutamate moiety, with or without a preceding linker moiety that bonds the peptide to the hydroxyl (—OH) of the fourth amino acid of the mitochondrial targeted peptide.

A prodrug compound may have the formula, where “n” is number comprising PVA polymer, as in the following example:

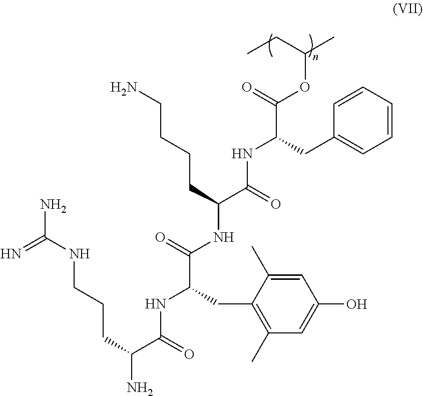

Also described herein are prodrug compounds of formula (VIII), H-d-Arg-DMT-Lys-Phe(-O)—R (designated as EY005-R):

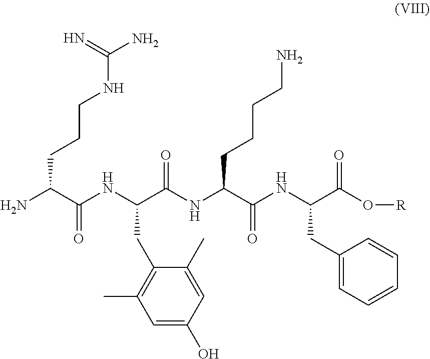

where the fourth amino acid is linked via ester bond to R, and R or —O—R is a conjugation moiety selected from: a C4-C30 lipid moiety, an C4-C30 straight-chain or branched aliphatic moiety, a 2-mer to 30-mer peptide moiety, or a pegylated moiety, or a carbohydrate moiety.

In some examples, the prodrug has the formula of: H-d-Arg-DMT-Lys-Phe(-O-)-nonpolar lipid (also referred to herein as EY005-nonpolar lipid). The nonpolar lipid may include one of several molecules, including octadecyl (where the —O—R is derived from stearyl alcohol) or hexadecyl (where the —O—R is derived from palmityl alcohol) or other comparable molecule as the conjugation moiety (example depicted in FIG. 16A). Prodrugs having a nonpolar lipid as the conjugation moiety are only one class of the prodrugs described herein that may be successfully with a lipid-based complexation agent, including complexation agents that are also nonpolar lipids. A nonpolar lipid is a hydrophobic molecule that is solid at temperatures between 27 to 50 degrees C., containing ketoacyl and isoprene groups inclusive but not restricted to fatty acids, glycerolipids, glycerophospholipids, sphingolipids, saccharolipids, polyketides (derived from condensation of ketoacyl subunits), sterol lipids and prenol lipids (derived from condensation of isoprene subunits).

In some examples, H-d-Arg-DMT-Lys-Phe (—OH) (EY005) manufactured as H-d-Arg-DMT-Lys-Phe (—O)—R, where the O—R group is a conjugation moiety that is a fatty acid or a fatty alcohol, having a carbon chain length of C4-C30, that is covalently linked to the hydroxyl group of the fourth amino acid, either directly or via a linker construct. One such example is H-d-Arg-DMT-Lys-Phe (—O)—R, in which the —O—R group is derived from stearyl alcohol (which is also depicted in FIG. 16A as EY005-stearyl, with esterase cleavage site is shown) that has been linked via the ester bond to the H-d-Arg-DMT-Lys-Phe(-OH). In another example, the prodrug is H-d-Arg-DMT-Lys-Phe (—O)—R, in which the —O—R group is derived from palmityl alcohol, which is depicted as EY005-hexadecyl, in which the —O—R group derived from palmityl alcohol has been linked via the ester bond to the H-d-Arg-DMT-Lys-Phe(-OH):

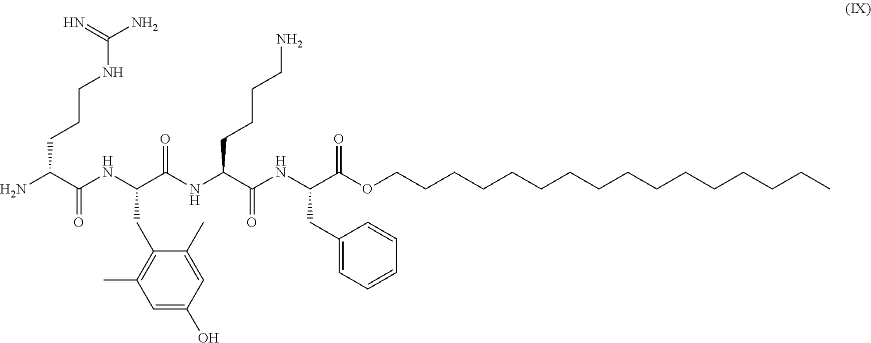 [SMILES: CCCCCCCCCCCCCCCCOC(=O)[C@H](Cc1ccccc1)NC(=O)[C@H](CCCCN)NC(=O)[C@H](Cc1c(C)cc(O)cc1C)NC(=O)[C@H](N)CCCNC(=N)N]

For example, also described herein are prodrug compounds having the formula: H-d-Arg-DMT-Lys-Phe(-O)-nonyl.

A prodrug compound may have the formula: H-d-Arg-DMT-Lys-Phe(-O)-decyl.

A prodrug compound may have the formula: H-d-Arg-DMT-Lys-Phe(-O)-undecyl.

A prodrug compound may have the formula: H-d-Arg-DMT-Lys-Phe(-O)-dodecyl.

A prodrug compound may have the formula: H-d-Arg-DMT-Lys-Phe(-O)-tridecyl.

A prodrug compound may have the formula: H-d-Arg-DMT-Lys-Phe(-O)-tetradecyl.

A prodrug compound may have the formula: H-d-Arg-DMT-Lys-Phe(-O)-pentadecyl.

A prodrug compound may have the formula: H-d-Arg-DMT-Lys-Phe(-O)-hexadecyl.

A prodrug compound may have the formula: H-d-Arg-DMT-Lys-Phe(-O)-heptadecyl.

A prodrug compound may have the formula: H-d-Arg-DMT-Lys-Phe(-O)-octadecyl.

A prodrug compound may have the formula: H-d-Arg-DMT-Lys-Phe(-O)-nonadecyl.

A prodrug compound may have the formula: H-d-Arg-DMT-Lys-Phe(-O)-icosyl.

A prodrug compound may have the formula: H-d-Arg-DMT-Lys-Phe(-O)-henicosyl.

A prodrug compound may have the formula: H-d-Arg-DMT-Lys-Phe(-O)-docosyl.

A prodrug compound may have the formula: H-d-Arg-DMT-Lys-Phe(-O)-tricosyl.

A prodrug compound may have the formula: H-d-Arg-DMT-Lys-Phe(-O)-tetracosyl.

A prodrug compound may have the formula: H-d-Arg-DMT-Lys-Phe(-O)-pentacosyl.

A prodrug compound may have the formula: H-d-Arg-DMT-Lys-Phe(-O)-hexacosyl.

A prodrug compound may have the formula: H-d-Arg-DMT-Lys-Phe(-O)-heptacosyl.

A prodrug compound may have the formula: H-d-Arg-DMT-Lys-Phe(-O)-octacosyl.

A prodrug compound may have the formula: H-d-Arg-DMT-Lys-Phe(-O)-nonacosyl.

A prodrug compound may have the formula: H-d-Arg-DMT-Lys-Phe(-O)-triacontyl.

A prodrug compound may have the formula: H-d-Arg-DMT-Lys-Phe(-O)-hentriacontyl.

A prodrug compound may have the formula: H-d-Arg-DMT-Lys-Phe(-O)-dotriacontyl.

A prodrug compound may have the formula: H-d-Arg-DMT-Lys-Phe(-O)-tritriacontyl.

A prodrug compound may have the formula: H-d-Arg-DMT-Lys-Phe(-O)-tetratriacontyl.

A prodrug compound may have the formula: H-d-Arg-DMT-Lys-Phe(-O)-pentatriacontyl.

In some examples, a peptide-based prodrug has the formula H-d-Arg-DMT-Lys-Phe (—O)—R, where R is a conjugation moiety comprising a cationic peptide or polypeptide molecule having of length 2-30 amino acids, where the individual peptides may be either distinct or repeats and is covalently linked to the hydroxyl group of the fourth amino acid, either directly or via a linker construct. One such example is H-d-Arg-DMT-Lys-Phe (—O)—R, where R is a polyarginine peptide (e.g., polyarginine hydrochloride azide):

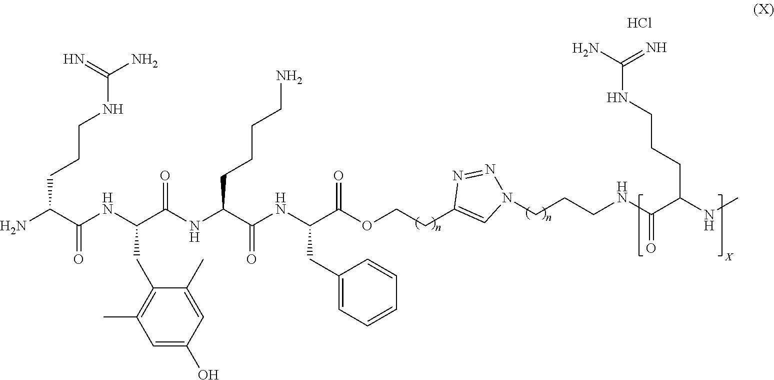

In some examples, a peptide-based prodrug has the formula H-d-Arg-DMT-Lys-Phe (—O)—R, where R is a conjugation moiety comprising an anionic peptide or polypeptide molecule having length 2-30 amino acids, where the individual peptides may be either distinct or repeats and is covalently linked to the hydroxyl group of the fourth amino acid, either directly or via a linker construct. One such example is H-d-Arg-DMT-Lys-Phe (—O)—R, where R is a polyglutamate peptide (e.g., polyglutamate azide):

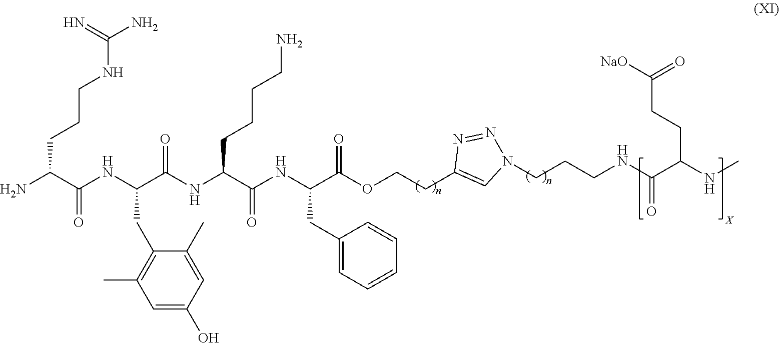

A prodrug compound may be H-d-Arg-DMT-Lys-Phe(-O)—R, wherein R is a 3-mer polyarginine moiety, with or without a preceding linker moiety that bonds the peptide to the hydroxyl (—OH) of the fourth amino acid of the mitochondrial targeted peptide (see FIG. 16C).

A prodrug compound may be H-d-Arg-DMT-Lys-Phe(-O)—R, wherein R is a 6-mer polyarginine moiety, with or without a preceding linker moiety that bonds the peptide to the hydroxyl (—OH) of the fourth amino acid of the mitochondrial targeted peptide.

A prodrug compound may be H-d-Arg-DMT-Lys-Phe(-O)—R, wherein R is an 8-mer polyarginine moiety, with or without a preceding linker moiety that bonds the peptide to the hydroxyl (—OH) of the fourth amino acid of the mitochondrial targeted peptide.

A prodrug compound may be H-d-Arg-DMT-Lys-Phe(-O)—R, wherein R is a 10-mer polyarginine moiety, with or without a preceding linker moiety that bonds the peptide to the hydroxyl (—OH) of the fourth amino acid of the mitochondrial targeted peptide.

A prodrug compound may be H-d-Arg-DMT-Lys-Phe(-O)—R, wherein R is a 3-mer polyglutamate moiety, with or without a preceding linker moiety that bonds the peptide to the hydroxyl (—OH) of the fourth amino acid of the mitochondrial targeted peptide (see FIG. 16B).

A prodrug compound may be H-d-Arg-DMT-Lys-Phe(-O)—R, wherein R is a 6-mer polyglutamate moiety, with or without a preceding linker moiety that bonds the peptide to the hydroxyl (—OH) of the fourth amino acid of the mitochondrial targeted peptide.

A prodrug compound may be H-d-Arg-DMT-Lys-Phe(-O)—R, wherein R is an 8-mer polyglutamate moiety, with or without a preceding linker moiety that bonds the peptide to the hydroxyl (—OH) of the fourth amino acid of the mitochondrial targeted peptide.

A prodrug compound may be H-d-Arg-DMT-Lys-Phe(-O)—R, wherein R is a 10-mer polyglutamate moiety, with or without a preceding linker moiety that bonds the peptide to the hydroxyl (—OH) of the fourth amino acid of the mitochondrial targeted peptide.

A prodrug compound may be H-d-Arg-DMT-Lys-Phe(-O)—R, wherein R is a 3-mer polyaspartate moiety, with or without a preceding linker moiety that bonds the peptide to the hydroxyl (—OH) of the fourth amino acid of the mitochondrial targeted peptide.

A prodrug compound may be H-d-Arg-DMT-Lys-Phe(-O)—R, wherein R is a 6-mer polyaspartate moiety, with or without a preceding linker moiety that bonds the peptide to the hydroxyl (—OH) of the fourth amino acid of the mitochondrial targeted peptide.

A prodrug compound may be H-d-Arg-DMT-Lys-Phe(-O)—R, wherein R is an 8-mer polyaspartate moiety, with or without a preceding linker moiety that bonds the peptide to the hydroxyl (—OH) the fourth amino acid of the mitochondrial targeted peptide.

A prodrug compound may be H-d-Arg-DMT-Lys-Phe(-O)—R, wherein R is a 10-mer polyaspartate moiety, with or without a preceding linker moiety that bonds the peptide to the hydroxyl (—OH) the fourth amino acid of the mitochondrial targeted peptide.

A prodrug compound may be H-d-Arg-DMT-Lys-Phe(-O)—R, wherein R is a 3-mer polyhistidine moiety, with or without a preceding linker moiety that bonds the peptide to the hydroxyl (—OH) of the fourth amino acid of the mitochondrial targeted peptide.

A prodrug compound may be H-d-Arg-DMT-Lys-Phe(-O)—R, wherein R is a 6-mer polyhistidine moiety, with or without a preceding linker moiety that bonds the peptide to the hydroxyl (—OH) of the fourth amino acid of the mitochondrial targeted peptide.

A prodrug compound may be H-d-Arg-DMT-Lys-Phe(-O)—R, wherein R is an 8-mer polyhistidine moiety, with or without a preceding linker moiety that bonds the peptide to the hydroxyl (—OH) of the fourth amino acid of the mitochondrial targeted peptide.

A prodrug compound may be H-d-Arg-DMT-Lys-Phe(-O)—R, wherein R is a 10-mer polyhistidine moiety, with or without a preceding linker moiety that bonds the peptide to the hydroxyl (—OH) of the fourth amino acid of the mitochondrial targeted peptide.

A prodrug compound may be H-d-Arg-DMT-Lys-Phe(-O)—R, wherein R is a 3-mer polylysine moiety, with or without a preceding linker moiety that bonds the peptide to the hydroxyl (—OH) of the fourth amino acid of the mitochondrial targeted peptide.

A prodrug compound may be H-d-Arg-DMT-Lys-Phe(-O)—R, wherein R is a 6-mer polylysine moiety, with or without a preceding linker moiety that bonds the peptide to the hydroxyl (—OH) of the fourth amino acid of the mitochondrial targeted peptide.

A prodrug compound may be H-d-Arg-DMT-Lys-Phe(-O)—R, wherein R is an 8-mer polylysine moiety, with or without a preceding linker moiety that bonds the peptide to the hydroxyl (—OH) of the fourth amino acid of the mitochondrial targeted peptide.

A prodrug compound may be H-d-Arg-DMT-Lys-Phe(-O)—R, wherein R is a 10-mer polylysine moiety, with or without a preceding linker moiety that bonds the peptide to the hydroxyl (—OH) of the fourth amino acid of the mitochondrial targeted peptide.

A prodrug compound may be H-d-Arg-DMT-Lys-Phe(-O)—R, wherein R is a polyethylene glycol (PEG) polymer, a pegylated peptide, or pegylated succinate including PEG polymers of linear, branched, Y-shaped, or multi-arm geometries.

In some examples, a prodrug has the formula H-d-Arg-DMT-Lys-Phe (—O)—R, where R is a conjugation moiety comprising a polyethylene glycol (PEG) polymer that is covalently linked to the hydroxyl group of Phe-OH, either directly or via a linker construct. One such example is H-d-Arg-DMT-Lys-Phe (—O)—R, where R is a PEG polymer moiety (as also depicted in FIG. 16D) where prodrug is EY005-PEG with esterase cleavage site as indicated):

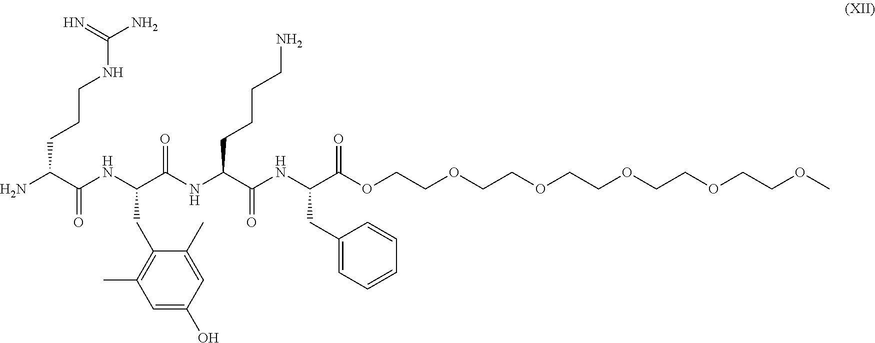 [SMILES: COCCOCCOCCOCCOCCOC(=O)[C@H](Cc1ccccc1)NC(=O)[C@H](CCCCN)NC(=O)[C@H](Cc1c(C)cc(O)cc1C)NC(=O)[C@H](N)CCCNC(=N)N]

In some examples, a prodrug has the formula H-d-Arg-DMT-Lys-Phe (—O)—R, where R is a conjugation moiety comprising a PEGylated peptide or protein that is covalently linked to the hydroxyl group of Phe-OH, either directly or via a linker construct. One such example is H-d-Arg-DMT-Lys-Phe (—O)—R, where R is a PEGylated peptide or protein.

In some examples, a prodrug has the formula H-d-Arg-DMT-Lys-Phe (—O)—R, where R is a conjugation moiety comprising a PEGylated peptide or protein that is covalently linked to the hydroxyl group of Phe-OH, either directly or via a linker construct. One such example is H-d-Arg-DMT-Lys-Phe (—O)—R, where R is a conjugation moiety comprising a PEGylated succinate that is covalently linked to the hydroxyl group of Phe-OH, either directly or via a linker construct. One such example is H-d-Arg-DMT-Lys-Phe (—O)—R, where R is a PEGylated succinate.

A prodrug compound may be H-d-Arg-DMT-Lys-Phe(-O)—R, wherein R is a carbohydrate moiety comprising carbohydrate molecule comprising a monosaccharide or oligosaccharide of 2 to 20 sugars which is covalently bound to the hydroxyl (—OH) of the fourth amino acid of the mitochondrial targeted peptide. These include but are not limited to monosaccharides or oligosaccharides comprising glucose, galactose, lactose, mannose, ribose, fucose, N-acetylgalactosamine, N-acetylglucosamine, N-acetyleneuraminic acid, or to any of their epimers or derivatives.

A prodrug compound may be (H-d-Arg-DMT-Lys-Phe(-O))ₙ—R, wherein R is a linker or multimerization domain which is convalently linked to multiple mitochondria targeting peptides to form dimers or multimers of the prodrug and n is equal to 2 to about 100. The multimerization domain may comprise one or more of: PEG, a PEG polymer, polyvinyl alcohol (PVA), or peptide.

One such example is H-d-Arg-DMT-Lys-Phe (—O)—R, where R is where R is a polyvinyl alcohol (PVA) that can link to one or more other mitochondrial targeted peptides at the hydroxyl group of the amino acid in the 4th position:

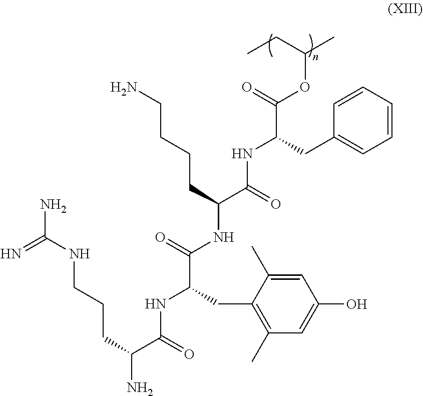

Complexation occurs in two physicochemical circumstances. In one case, complexation occurs with noncovalent interactions between individual molecules (e.g., receptor-ligand interactions). This type of complexation is termed molecular complexation. The second circumstance involves a molecule of a chemical substance (in this case, molecule of drug) that noncovalently binds or adsorbs to a surface of a particulate (in this case, a complexation agent). This type of complexation is termed particulate complexation, and different particulate adsorbents, or complexation agents, have different sorptive properties based on size and shape of particulate, functional groups present at the surface, and the surface irregularity and porosity of the particulate. The utility of particulate complexation has been recognized in other disciplines, including soil sciences, wherein a chemical adsorbent (e.g., alumina, silica gel, activated charcoal) interacts with specific chemicals (frequently contaminants) in soil and the adsorption-desorption properties are particularly important for nutrients, fertilizers, defoliants, insecticides. In the oil and hydrocarbon industry, wherein adsorbents (e.g., polypropylene, vermiculite, perlite, polyethylene, others) are used to clean oil spills or to remove residual oil from drilling and fracking equipment; and industrial coatings (e.g., zeolite, silica gel, aluminum phosphate), wherein adsorbents are used to bind chemical substances for various purposes (i.e., lubrication, surface cooling).

In medical applications, adsorbents are used for the treatment of acute poisoning by ingestion (e.g., activated charcoal, calcium polystyrene sulfate, aluminum silicate) where the adsorbent binds the toxin to limit adsorption from the gut into systemic circulation.

In the pharmaceutical industry, principles of adsorption complexation best known in the pharmaceutical sciences in the form of drug binding to plasma proteins in the blood, drug coatings on solid scaffolds for in situ drug release (e.g., drug-eluting stents), and affixation of excipients to insoluble drugs in order to improve oral bioavailability and absorption.

However, there is no extended release drug delivery system for ocular drug delivery that is based on complexation systems, or that utilize drug-adsorption noncovalent interactions with complexation agents, to regulate release of drug from the drug delivery system implant into ocular tissue.

The compositions and methods described herein may utilize particulate complexation, wherein complexation agents thus are chemicals compatible with ocular tissues that, when formulated as an irregularly shaped particulates, have the capacity of noncovalently binding MTT-prodrug, forming MTT-prodrug-complex particulates. This system leverages complexation chemistry systems without changing the properties or activity of the drug itself by utilizing a prodrug strategy. One or more MTT-prodrug-complex particulates are incorporated and admixed into a hydrophobic dispersal medium to form a stable multiphasic colloidal suspension, that is safely delivered into and around the eye. This complexation-based XRDDS regulates the diffusion and release of free MTT-prodrug diffusion into the tissue, where tissue (vitreous) esterases cleave and release free MTT drug or the ester cleaves by hydrolysis, providing an integrated mechanism for regulation of release of drug into tissue.

The conjugation moiety of the MTT-prodrug is specifically chosen for its ability to complex, or form noncovalent interactions, with one or more particulate complexation agents to form “drug-complex” particulates, which are subsequently combined and dispersed within a selected dispersal medium to form a stable multiphasic colloidal suspension. Complexation agents are selected from one of six classes of chemical substances, including fatty acid, organic compounds that can form keto-enol tautomer, charged phospholipid, charged protein, nucleic acid, and polysaccharides.

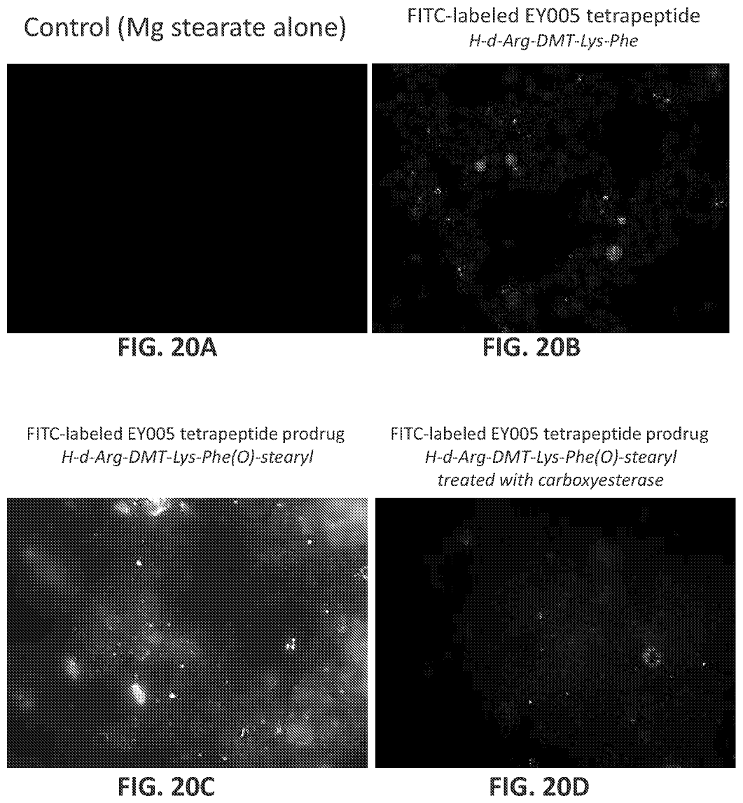

The compositions and methods described herein disclose a new property, not previously recognized, of these six classes of chemical substances, fatty acid, organic compounds that can form keto-enol tautomer, charged phospholipid, charged protein, nucleic acid, and polysaccharides, that, when in the form of an irregularly shaped particulate with irregular surface, can serve as an effective complexation agent for MTT-prodrugs. The criteria for complexation agent includes the following four features: (1) fluorescein-labeled binds to the particulate via the conjugation moiety and not the MTT itself, and this is demonstrable by microscopy imaging (see FIGS. 20C and 20D, 21C and 21D, 22C and 22D and 23C and 23D); (2) when particulate of substance is added to a solution of MTT-prodrug, upon centrifugation and pulldown of the particulates, pharmacologically significant quantities of drug are observed to be complexed to the particulates (see Table 2, below); (3) drug particulate-complexes, when resuspended in appropriate dispersal medium, demonstrate partial release of drug, which can be demonstrated by Kd or unbound-bound fraction of drug for a given MTT-prodrug-complexation agent pair in a particular dispersal medium (see Table 2, below); and (4) the drug-particulate complexes provide a useful pharmacokinetic release profile from the dispersal medium (see FIG. 25B). Collectively, these four properties define a complexation agent and enable the presently described complexation-based XRDDS.

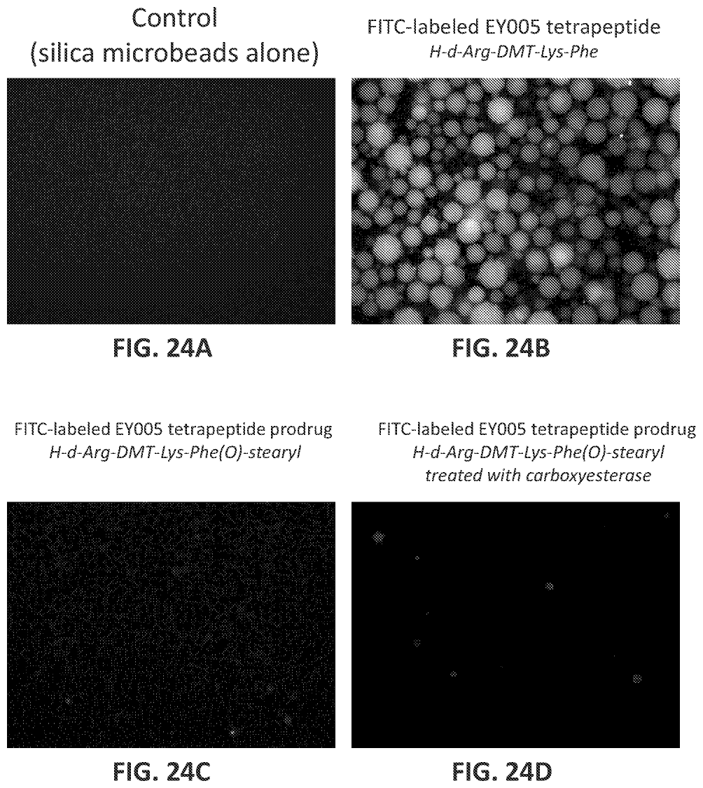

In contrast, spherical particulates with a spherical smooth surface and non-reactive coating, including for example silicone beads, latex beads, and certain polymeric microparticulates, fail to form complexes with MTT-prodrug (FIG. 24C), and therefore may be excluded from the compositions and methods described herein.

One class of complexation agents is fatty acid, which is a carboxylic acid with an aliphatic chain, which may be either saturated or unsaturated, and may be in the form of a salt or ester. For example, the fatty acid may have a chemical formula of CH3(CH2)ₙCOOH where n is equal to between 4 and 30. The fatty acid may comprise one of: Tetradecanoic acid, pentadecanoic acid, (9Z)-hexadecenoic acid, Hexadecanoic acid, Heptadecanoic acid, Octadecanoic acid, (9Z,12Z)-octadeca-9,12-dienoic acid, (9Z,12Z,15Z)-octadeca-9,12,15-trienoic acid, (6Z,9Z,12Z)-octadeca-6,9,12-trienoic acid, (5E,9E,12E)-octadeca-5,9,12-trienoic acid, (6Z,9Z,12Z,15Z)-octadeca-6,9,12,15-tetraenoic acid, (Z)-octadec-9-enoic acid, (11E)-octadec-11-enoic acid, (E)-octadec-9-enoic acid, nonadecanoic acid, eicosanoic acid etc.). The fatty acid may be an unbranched fatty acid between C14 and C20. The fatty acid may be a saturated fatty acid comprising one of: myristic acid (tetradecanoic acid), palmitic acid (hexadecanoic acid), stearic acid (octadecanoic acid), arachidic acid (eicosanoic acid). Specific examples of salt form fatty acids include magnesium stearate, magnesium palmitate, calcium stearate, calcium palmitate, and others.

For example, the complexation agent may be a C18 fatty acid (e.g., stearic acid, or octadecanoic acid). In some examples, the MTT described herein is covalently linked (e.g., via an ester bond) to a conjugation moiety comprising stearic acid or stearyl alcohol (e.g., EY005-octadecyl, or EY005-stearyl as shown in FIG. 16A), and the XRDDS may include stearic acid or a salt form thereof (magnesium stearate) as a complexation agent.

One class of complexation agents is organic compounds that can form keto-enol tautomers. Tautomers refer to molecules capable of undergoing chemical equilibrium between a keto form (a ketone or an aldehyde) and an enol form (an alcohol). Usually, a compound capable of undergoing keto-enol tautomerization contains a carbonyl group (C═O) in equilibrium with an enol tautomer, which contains a pair of doubly bonded carbon atoms adjacent to a hydroxyl (—OH) group, C═C—OH as depicted herein:

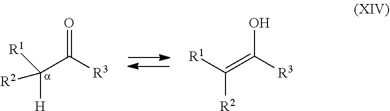

The relative concentration of the keto and enol forms is determined by the chemical properties of the specific molecule and the chemical microenvironment, including equilibrium, temperature or redox state. Organic compounds capable of keto-enol tautomerization include but are not limited to phenols, tocofersolan, tocopherols, quinones, ribonucleic acids, and others.

One class of complexation agents is charged phospholipid. In general, phospholipids consist of a glycerol molecule, two fatty acids, and a phosphate group that is modified by an alcohol, wherein the polar head of the phospholipid is typically negatively charged. Examples include lecithin, phosphatidylcholine, phosphatidylethanolamine, phosphatidylserine, different phospholipids in oil, and many others, which may be used individually or in combination to serve as complexation agents. Anionic phospholipids may comprise one of: phosphatidic acid, phophatidyl serine, sphingomyelin or phosphatidyl inositol. In some instances, synthetic, ionizable phospholipids with positive charge can manufactured, including but not limited to examples such as DLin-MC3-DMA. Additional cationic phospholipids may comprise one of: cationic triesters of phosphatidylcholine; 1,2-dimyristoylsn-glycerol-3-phosphocholine (DMPC); 1,2-dioleoyl-sn-glycerol-3-phosphocholine (DOPC); 1,2-bis(oleoyloxy)-3-(trimethylammonio)propane (DOTAP); 1,2-dioleoyl-sn-glycerol-3-phosphoethanolamine (DOPE); 1,2-dipalmitoyl-sn-glycerol-3-phosphocholine (DPPC); 1,2-dioleoyl-sn-glycerol-3-ethylphosphocholine (EDOPC);1,2-dimyristoyl-sn-glycerol-3-ethylphosphocholine (EDMPC); 1,2-dipalmitoyl-sn-glycerol-3-ethylphosphocholine (EDPPC). In pharmaceutical sciences, phospholipids have been used for drug formulation and delivery applications to improve bio-availability, reduced toxicity, and improved cellular permeability. However, in the compositions and methods described herein, phospholipids may be used as a complexation agent particulate to noncovalently bind the conjugation moiety of the MTT-prodrug and form MTT-prodrug complex particulates for the purpose of regulating free MTT-prodrug in the dispersal medium of the stable multiphasic colloidal suspension in which the MTT-prodrug complex particulates are incorporated and dispersed therein.

In some examples, an anionic phospholipid may form noncovalent complexaiton with a cationic conjugation moiety of an MTT-prodrug. A cationic phospholipid may form noncovalent complexaiton with an anionic conjugation moiety of an MTT-prodrug.

One class of complexation agents is charged protein. Proteins are large biomolecules and macromolecules that comprise one or more long changes of amino acid residues. Amino acids that make up proteins may be positive, negative, neutral, or polar in nature, and collectively, the amino acids that comprise the protein give it its overall charge. A variety of proteins, based on size, molecular weight, ability to readily form particulates, and compatibility with ocular tissues could serve as complexation agents. The charge of the protein will determine its compatibility with a specific MTT-prodrug, such that negatively charged proteins will readily complex with positively charged conjugation moiety of MTT-prodrug, while positively charged proteins (e.g., Arg-Gln-Ile-Arg-Arg-Ile-Ile-Gln-Arg-NH₂ and synthetic peptides with positive charge) will readily complex with negatively charged conjugation moiety of MTT-prodrug. Examples of proteins that could serve as complexation agents include albumin and collagen.

One class of complexation agents is nucleic acids, biopolymer macromolecules comprising nucleotides, comprising a 5-carbon sugar, a phosphate group, and a nitrogenous base. The importance of nucleic acids for biologic function and encoding genetic information is well established. However, nucleic acids also have a variety of applications, including nucleic acid enzymes (e.g., carbon nanomaterials), aptamers (e.g., for formation of nucleic acid nanostructures and therapeutic molecules that function in an antibody-like fashion), and aptazymes (e.g., which can be used for in vivo imaging). In pharmaceutical sciences, specially engineered nucleic acids have been considered and applied for use in carrier-based systems in which the nucleic acid serves as a carrier system for various types of drugs. However, in the compositions and methods described herein, nucleic acids may not be considered a carrier system but rather as a complexation agent, as they are highly negatively charged and thus, formulated as a particulate, could then serve as a complexation agent for positively charged conjugation moiety of the MTT-prodrug.

One class of complexation agent is polysaccharides, long chain polymeric carbohydrates comprising monosaccharide units bound together by glycosidic linkages. Frequently, these are quite heterogenous, containing slight modifications of the repeating monosaccharide unit. Depending on structure, they can be insoluble in water. Complexation of polysaccharide particulate complexation agents to other molecules, in this case, various MTT-prodrugs, can occur through various electrostatic interactions and is influenced by charge density of conjugation moiety of MTT-prodrug and polysaccharide, ratio of polysaccharide complexation agent to MTT-prodrug, ionic strength, and other properties. Examples of polysaccharides that could serve as complexation agents include a ringed polysaccharide molecule, cyclodextrins, a clathrate, starch, cellulose, pectins, or acidic polysaccharides (polysaccharides that contain carboxyl groups, phosphate groups, or other similarly charged groups.

The complexation agent may be a compound containing metal ions.

In any of these therapeutic compositions an ionic coordination complexation may occur around a central ion forming extensive noncovalent interactions. The central ion may be a central metal ion comprising one of: copper, iron, zinc, platinum, or lithium.

Ionic coordination complexation is a chemical complexation process around a central ion, usually a metal, capable of forming extensive noncovalent electrostatic interactions with a wide range of chemical substances. This is one of the most common chemical processes in nature. The avidity of binding is variable amongst different coordination ions, some of which may be nearly irreversible while others manifest relatively labile binding. Central metal ions include copper, iron, zinc, platinum, lithium, others. Three classes that can serve as a complexation agent for drug delivery are chelators (EDTA), complexation to certain specific metals (platinum, lithium, lanthanum) and molecules with metalloprotein elements (hemoglobin, porphyrin, superoxide dismutase, and others with zinc or copper binding domains).

The complexation agent may comprise a chelator configured for complexation to a metal, a metalloprotein, or a superoxide dismutase (SOD). The complexation agent may comprise a chelator configured for complexation to one or more of: platinum, lithium, lanthanum, hemoglobin, porphyrin, zinc binding domains, or superoxide dismutase (SOD).

In the compositions and methods described herein, the conjugation moiety of the MTT-prodrug has specific avidity for, and complexes with, a given complexation agent, forming an MTT-prodrug-complex particulate. This avidity can be measured as Kd, the unbound-bound fraction of an MTT-prodrug for a given MTT-prodrug-complex particulate in a selected dispersal medium. The binding of the conjugation moiety of the MTT-prodrug to a particular complexation agent thus serves to limit the free drug available for release from a given dispersal medium.

Thus, in the complexation-based XRDDS comprising one or more MTT-prodrug-complex particulates incorporated into a hydrophobic dispersal medium, rather than use of complexation to improve bioavailability, formulations of the XRDDS use complexation to limit free, unbound MTT-prodrug available for release from a given dispersal medium of the multiphasic colloidal suspension.

As described herein, one example of EY005-prodrugs includes EY005-stearyl (FIG. 16A). As EY005 is linked via ester bond to stearyl alcohol, the resultant EY005-stearyl prodrug is hydrophobic, as compared to the unmodified MTT EY005, which is highly hydrophilic. EY005-stearyl readily forms noncovalent complex with solid lipid particulate complexation agents, such as magnesium stearate, to form MTT-prodrug-magnesium stearate particulates. The high avidity interaction between the hydrophobic, long-chain fatty alcohol of the conjugation moiety of this MTT-prodrug and the particulate complexation agent magnesium stearate serves to bind the MTT-prodrug and limits the free, unbound MTT-prodrug that is available for release from the dispersal medium in which the MTT-prodrug-complex particulate is dispersed.

Another specific example of EY005-prodrugs includes EY005-tri-arginine (triArg) (FIG. 16C), wherein EY005 is linked via ester bond to arginine trimer/tripeptide, a positively charged peptide conjugation moiety that readily forms noncovalent complex with negatively charged particulate complexation agents to form MTT-prodrug-complex particulates. The high avidity interaction between the positively conjugation moiety of this and the negative charge of the particulate complexation agent serves to bind MTT-triArg prodrug and limits the free, unbound MTT-prodrug that is available for release from the dispersal medium in which the MTT-prodrug-complex particulate is dispersed.

Another specific example of EY005-prodrugs includes EY005-tri-glutamate (triGlu) (FIG. 16B), wherein EY005 is linked via ester bond to glutamate trimer/tripeptide, a negatively charged peptide conjugation moiety that readily forms noncovalent complex with positively charged particulate complexation agents to form MTT-prodrug-complex particulates. The high avidity interaction between the negatively charged conjugation moiety of this and the positive charge of the particulate complexation agent serves to bind MTT-triGlu prodrug and limits the free, unbound MTT-prodrug that is available for release from the dispersal medium in which the MTT-prodrug-complex particulate is dispersed.

In examples in which the conjugation moiety of EY005-prodrug is a pegylated peptide, such as EY005-polyethylene glycol (PEG) (FIG. 16D), he complexation agent may form noncovalent interactions with the PEG or PEGylated conjugation moiety based on its size and charge.

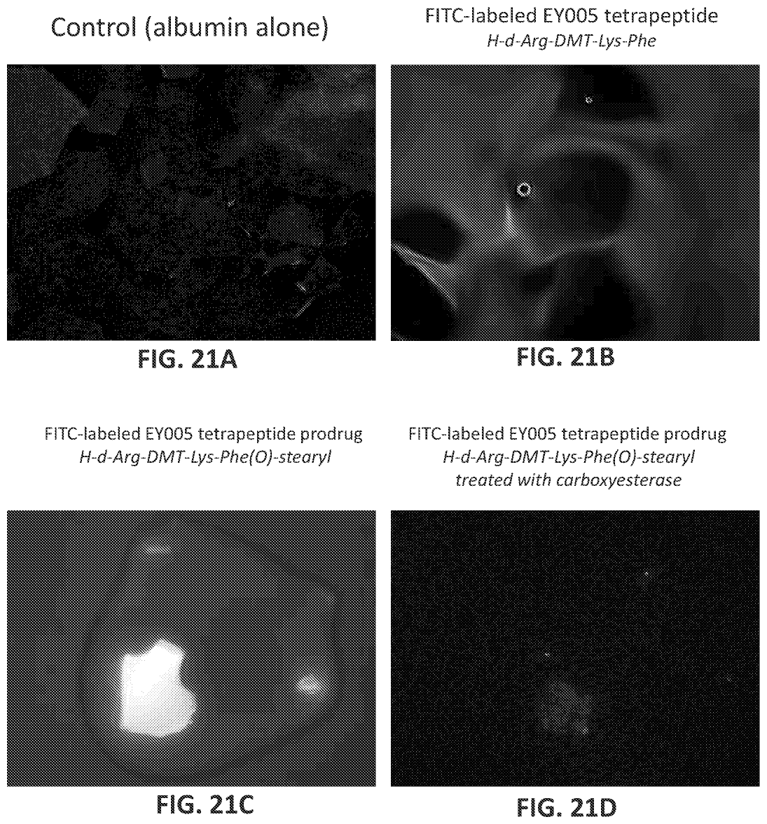

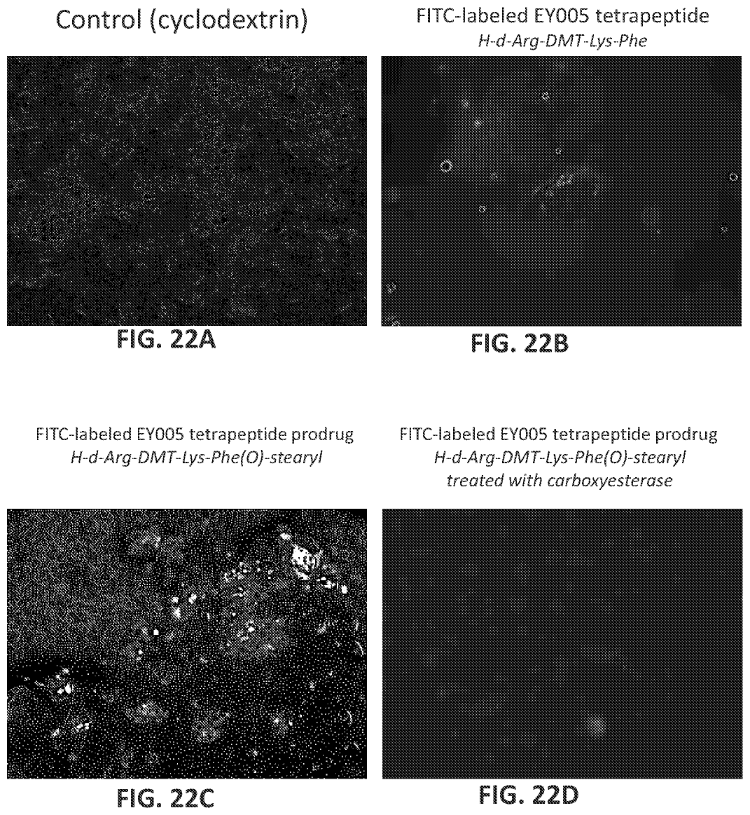

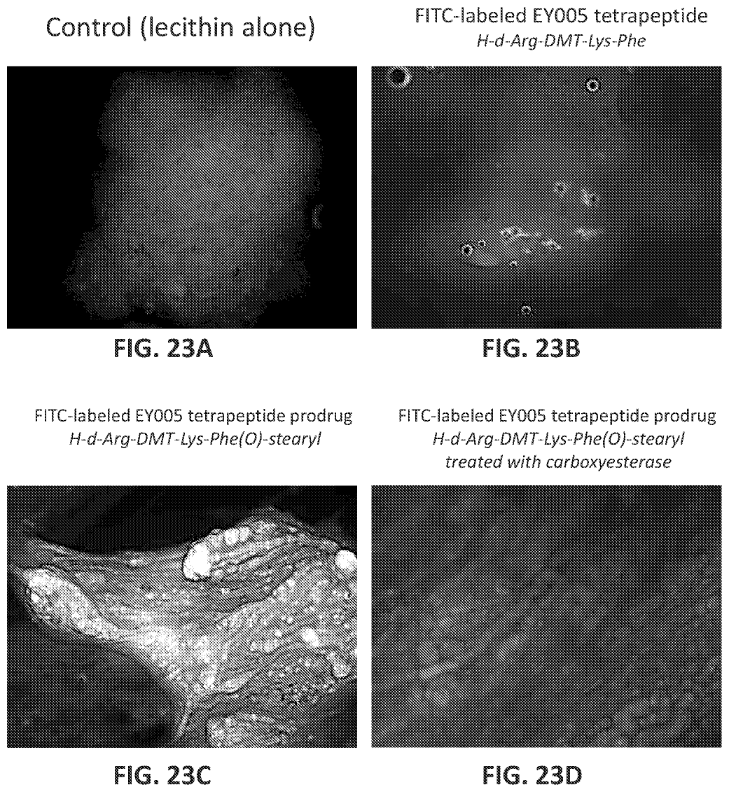

As described herein, formation of MTT-prodrug-complex particulates can be verified experimentally by direct visualization. For example, the MTT-prodrug EY005-stearyl was fluorescently labeled with fluorescein isothiocyanate (FITC) and admixed with different complexation agents. The resultant mixture was then visualized under direct fluorescence microscopy. Using this approach, FITC-labeled EY005-stearyl was observed to form drug-complex particulates with several different complexation agents: magnesium stearate (as previously described, and as expected); albumin, a large, charged carrier protein; and cyclodextran, a large cyclic carbohydrate molecule (FIG. 20C, FIG. 21C, FIG. 22C, FIG. 23C). In contrast, FITC-labeled EY005-stearyl was not observed to form drug-complex particulates with silica microbeads (FIG. 24C), indicating the process of complexation and drug-complex particulate formation is highly dependent on favorable noncovalent interaction between drug and complexation agent.

Further, this noncovalent interaction is specifically mediated by the conjugation moiety of the MTT-prodrug. FITC-labeled EY005-stearyl that had been admixed with complexation agent was treated with an aqueous solution of carboxyesterase (0.1 μg/mL) to hydrolyze the ester bond of the prodrug, releasing the fluorescent peptide. Complexed particulates were no longer fluorescently labeled by microscopy (FIG. 20D, FIG. 21D, FIG. 22D, FIG. 23D), affirming that complexation of the prodrug is specifically mediated by the conjugation moiety of the MTT-prodrug.

As described herein, formation of drug-complex particulates in which the complexation agent has high avidity for the drug can be quantified and verified experimentally. For example, the MTT-prodrug EY005-stearyl was admixed with known quantities of selected individual complexation agents. The EY005-stearyl-complexation agent mixture was then added to an appropriate dispersal medium (in this case, methyl laurate), and centrifuged to “pull down” or separate EY005-stearyl bound to complexation agent from unbound prodrug present in the dispersal medium. HPLC analysis of pulled down particulates and dispersal medium from EY005-stearyl content determined the fraction of MTT-prodrug that is bound to the complexation agent (binding capacity) and calculation of the Kd value, the unbound to bound coefficient, for the MTT-prodrug/complexation agent pair. Using this type of assay, binding capacity and Kd values can be generated to identify the unbound to bound drug ratio for specific MTT-prodrug/complexation agent pairs in a selected dispersal medium, as shown

TABLE 2

| | μg EY005-stearyl complexed | |
|---|---|---|
| Complexation agent | per mg complexation agent | Calculated Kd |
| Magnesium stearate | 1.1 | 8.59 |
| Cyclodextrin gamma | 2.3 | 3.58 |
| Lecithin | 9.5 | 0.11 |
| Albumin | 4.5 | 1.34 |
| Silica microbeads | 0.2 | 51.72 |

Table 2 illustrates an example of a EY005-stearyl prodrug solubilized in methyl laurate in which various complexation agents were added, mixed, and incubated for 1 hour. The quantity of EY005-stearyl prodrug complexed with each complexation agent was determined by HPLC. Kd values were calculated as [unbound]/[bound] fraction prodrug under each condition. Binding capacity was calculated as the μg EY005-stearyl complexed per mg complexation agent. These data demonstrated variable degrees of complexation with each class of complexation agent.

Intravitreal administration of MTT-prodrugs alone, without an XRDDS, is insufficient to provided extended, durable release of drug at the retina. The free bioactive MTT is immediately cleaved from the prodrug molecule upon exposure to naturally occurring enzymes (e.g., esterases) present in the vitreous and ocular tissues, and the free bioactive tetrapeptide drug is subject to the same short vitreous half-life, 5-6 hours, as the unmodified tetrapeptide drug, leading to rapid clearance from the eye following a single IVT administration.

Extended release drug delivery systems (XRDDS) are devices, formulations, or other systems used in the design, manufacture, and administration of specific drugs, in a manner that regulates release kinetics optimized for specific therapeutic goal for a particular route of administration.

The extended release drug delivery system (XRDDS) described herein is comprising MTT-prodrug admixed with one or more particulate complexation agents to form “drug-complex” particulates, which are combined and dispersed within a selected dispersal medium to form a stable multiphasic colloidal suspension. The complexation-based XRDDS is herein referred to as “Mito XR,” or the implant, which is administered by intravitreal (IVT) or periocular (e.g., subconjunctival, sub-Tenon's) routes of administration. A variety of different complexation agents and different types of dispersal medium may be used for formation of the stable multiphasic colloidal suspension, as described herein.

Colloids are mixtures in which particulate substances are stably dispersed within a vehicle, called a dispersal medium, but do not settle or migrate. This differentiates a colloid from a suspension in which the particles settle within the suspension vehicle due to gravity. Typical particulate size for colloids is in the nanometer range. In colloids, the defining characteristic of the mixture is that particulates remain stably dispersed with minimal settling or migration. Colloid mixture in which particulates are dispersed in a liquid is called a “sol.” Colloid mixtures in which particulates are dispersed in a solid or semisolid is called a “solid colloid.” Colloid mixtures in which particulates are stably dispersed in a viscous semi-solid or solid dispersal medium have not been given a defined named. Herein, we refer to stably dispersed particulates as “colloidal suspension,” in reference to the larger sized stably dispersed particulates rather than nanoparticulates in a typical colloid. In the compositions and methods described herein, the dispersal medium may be a hydrophobic dispersal medium that facilitates a stable colloidal suspension. A multiphasic colloidal suspension is a suspension in which the drug substance is present in more than one phase, including free drug, drug-drug aggregates, and most importantly, drug noncovalently bound to complexation agent particulates. A multiphasic colloidal suspension may incorporate MTT-prodrug as the drug substance.

The complexation agent described herein may be noncovalently complexed with the conjugation moiety of the prodrug and incorporated and stably dispersed within a dispersal medium, which forms the multiphasic colloidal suspension.

Complexation of MTT-prodrugs to particulate complexation agents within the dispersal medium serves to limit the release of free MTT-prodrug into the dispersal medium. While the dispersal medium restricts access of water to the MTT-prodrug-complex particulates, free, unbound MTT-prodrug substance diffuses freely within the dispersal medium, and the dispersal medium does not retain the free, unbound drug, which can diffuse out of the multiphasic colloidal suspension.

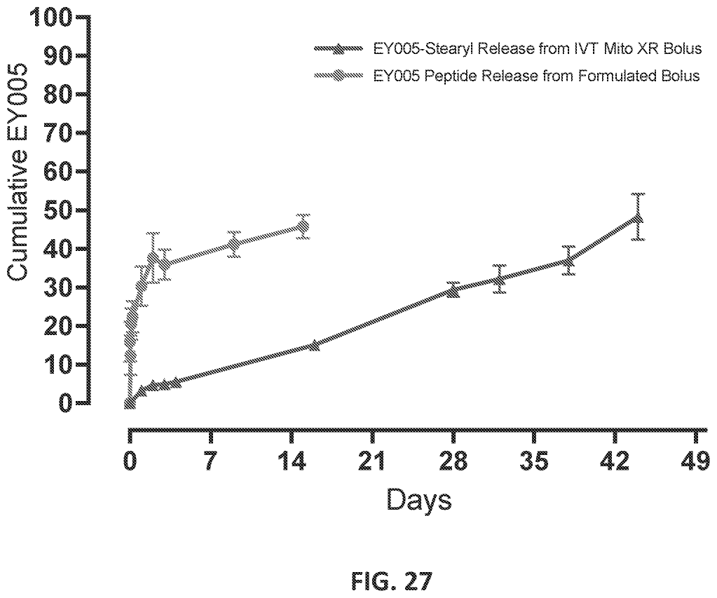

Incorporation of an unmodified MTT (e.g., H-d-Arg-DMT-Lys-Phe) into the complexation-based extended release drug delivery system (e.g., in biodegradable tube formulation) described herein is insufficient to provide durable extended release of drug, as compared to incorporation of MTT-prodrug (e.g., H-d-Arg-DMT-Lys-Phe-O-stearyl) when measured by in vitro assay of in sink conditions or by in vivo release into the vitreous and retinal tissues following intravitreal injection (FIGS. 27 and 29). In such examples, the unmodified tetrapeptide drug is rapidly released from the implanted drug delivery system and cleared from the eye in a short time (e.g., within a couple of weeks), due to a lack of complexation interaction between the unmodified MTT and selected complexation agents of the drug delivery system.

The prodrug serves a specific purpose in the complexation based XRDDS to optimize pharmacokinetics and enable sustained release following administration. The conjugation moiety of the prodrug forms noncovalent complexation interactions with selected complexation agent(s), serving to limit the amount of free drug that is available for release from the Mito XR implant. Specific conjugation moieties and specific complexation agents can be designed and selected to optimize avid nonconvalent interactions for a given drug-complexation agent pair, wherein the two are admixed for form “drug-complex” particulates and drug release rate from a given drug-complex particulate can be measured by in vitro assay of drug release into “in sink” conditions. The relative avidity of noncovalent interactions for a given MTT-prodrug-complex particulate can be measured by “Kd,” defined as the ratio of unbound to bound prodrug in a specified release assay. One or more sets of MTT-prodrug-complex particulates are incorporated and dispersed within a specific dispersal medium to form the multiphasic colloidal suspension that constitutes the XRDDS. The binding of the conjugation moiety of the MTT-prodrug to a particular complexation agent thus serves to limit the free drug available for release from a given dispersal medium (FIG. 25B). In general, the complexation-based XRDDS is formulated as an implant modality that can be administered by local ocular administration to achieve prespecified release kinetics of the active drug, designed and optimized for the specific therapeutic goal.

The dispersal medium as defined herein is a hydrophobic liquid that forms a stable multiphasic colloidal suspension when MTT-prodrug-complex particulates are incorporated and admixed into the dispersal medium.

The compositions and methods described herein discloses new previously unrecognized properties of certain oils that allow them to serve as effective dispersal medium. These include hydrophobicity, high starting viscosity, and other properties that allow it to form a stable multiphasic colloidal suspension when admixed with MTT-prodrug-complex particulates. The criteria that define a stable multiphasic colloidal suspension include uniform mixture and distribution of the MTT-prodrug-complex particulates without settling, separation, or dissociation of the particulates for the prespecified duration of the implant's lifetime, after exposure to an ocular physiologic environment in vitro (i.e., 37° C., buffered saline, vitreous enzymes, dilute serum) or in vivo when injected into the eye. The stability is also dependent on the relative percentage of MTT-prodrug-complex particulates to oil (weight to weight) and the size and mass of the particulates.

The dispersal medium of the multiphasic colloidal suspension stably disperses the MTT-prodrug-complex particulates and restricts access of water from surrounding tissue to the particulates within the XRDDS.

Four classes of oils that meet these criteria for formation of a stable multiphasic colloidal suspension include saturated fatty acid methyl esters, unsaturated fatty acid methyl esters, saturated fatty acid ethyl esters, or unsaturated fatty acid ethyl esters. A dispersal medium can be an individual oil from one of these classes or can be designed as a mixture of oils with different viscosity values that are specifically designed and admixed to achieve the desired goal of a stable colloidal suspension.

The saturated fatty acid methyl esters may comprise: methyl acetate, methyl propionate, methyl butyrate, methyl pentanoate, methyl hexanoate, methyl heptanoate, methyl octanoate, methyl nonanoate, methyl decanoate, methyl undecanoate, methyl dodecanoate (methyl laurate), methyl tridecanoate, methyl tetradecanoate, methyl 9(Z)-tetradecenoate, methyl pentadecanoate, methyl hexadecanoate, methyl heptadecanoate, methyl octadecenoate, methyl nonadecanoate, methyl eicosanoate, methyl heneicosanoate, methyl docosanoate, methyl tricosanoate, and others.

The saturated fatty acid ethyl esters may comprise: ethyl acetate, ethyl propionate, ethyl butyrate, ethyl pentanoate, ethyl hexanoate, ethyl heptanoate, ethyl octanoate, ethyl nonanoate, ethyl decanoate, ethyl undecanoate, ethyl dodecanoate (ethyl laurate), ethyl tridecanoate, ethyl tetradecanoate, ethyl 9(Z)-tetradecenoate, ethyl pentadecanoate, ethyl hexadecanoate, ethyl heptadecanoate, ethyl octadecenoate, ethyl nonadecanoate, ethyl eicosanoate, ethyl heneicosanoate, ethyl docosanoate, ethyl tricosanoate.

The unsaturated fatty acid methyl esters may comprise: methyl 10-undecenoate, methyl 11-dodecenoate, methyl 12-tridecenoate, methyl 9(E)-tetradecenoate, methyl 10(Z)-pentadecenoate, methyl 10(E)-pentadecenoate, methyl 14-pentadecenoate, methyl 9(Z)-hexadecenoate, methyl 9(E)-hexadecenoate, methyl 6(Z)-hexadecenoate, methyl 7(Z))-hexadecenoate, methyl 11(Z)-hexadecenoate, and so on for various unsaturated methyl esters, including but not limited to various methyl tricosenoate molecule entities.

The unsaturated fatty acid ethyl esters may comprise: ethyl 10-undecenoate, ethyl 11-dodecenoate, ethyl 12-tridecenoate, ethyl 9(E)-tetradecenoate, ethyl 10(Z)-pentadecenoate, ethyl 10(E)-pentadecenoate, ethyl 14-pentadecenoate, ethyl 9(Z)-hexadecenoate, ethyl 9(E)-hexadecenoate, ethyl 6(Z)-hexadecenoate, ethyl 7(Z))-hexadecenoate, ethyl 11(Z)-hexadecenoate, and so on for various unsaturated ethyl esters, including but not limited to various ethyl tricosenoate molecule entities.

In contrast, certain other oils and viscous substances including silicone oil, viscous gelatin, and viscous proteoglycan fail to form a stable multiphasic colloidal suspension or rapidly decompensate when exposed to a physiologic ocular microenvironment (e.g., 37° C., buffered saline, vitreous enzymes, dilute serum) or in vivo when injected into the eye.

Thus, the present extended release drug delivery system is novel and is and differentiated from previously conceived and designed systems because it instead utilizes the chemistry of complexation systems specifically for sustained release drug delivery to the eye, a method and approach for which there is no existing prior art for ocular drug delivery. The present system uses complexation of a drug onto one or more complexation agent(s) as a method to restrict diffusion of the drug and to regulate the kinetics of drug release into ocular tissue in a bioerodible modality, device, or formulation.

In some examples, cleavage and release of the free bioactive mitochondrial targeted tetrapeptide can be assessed following in vivo injection of the implant containing the prodrug within the extended release drug delivery system into the vitreous cavity or periocular tissues of a preclinical animal model (e.g., mouse, rat, rabbit, pig, etc.) (FIG. 29), wherein ocular tissue is recovered, and analytic methods such as HPLC or mass spectrometry can be used to calculate the amount of free bioactive mitochondrial targeted tetrapeptide and intact prodrug, at various timepoints after in vivo injection.

In one example, EY005-stearyl admixed with magnesium stearate (solid fatty acid) complexation agent and EY005-stearyl is admixed with alpha-tocopherol (keto-enol tautomer) complexation agent, and both drug-complex particulate pairs are incorporated into methyl laurate to form the stable multiphasic colloidal suspension, or Mito XR bolus implant.

In in vitro kinetics studies, this pilot formulation of Mito XR achieved zero-order (i.e., linear) kinetics of EY005 bioactive tetrapeptide, achieving the desired durability of drug release of three months, with free bioactive MTT within the dispersal medium released from the implant into the ocular physiologic environment (see FIG. 27).

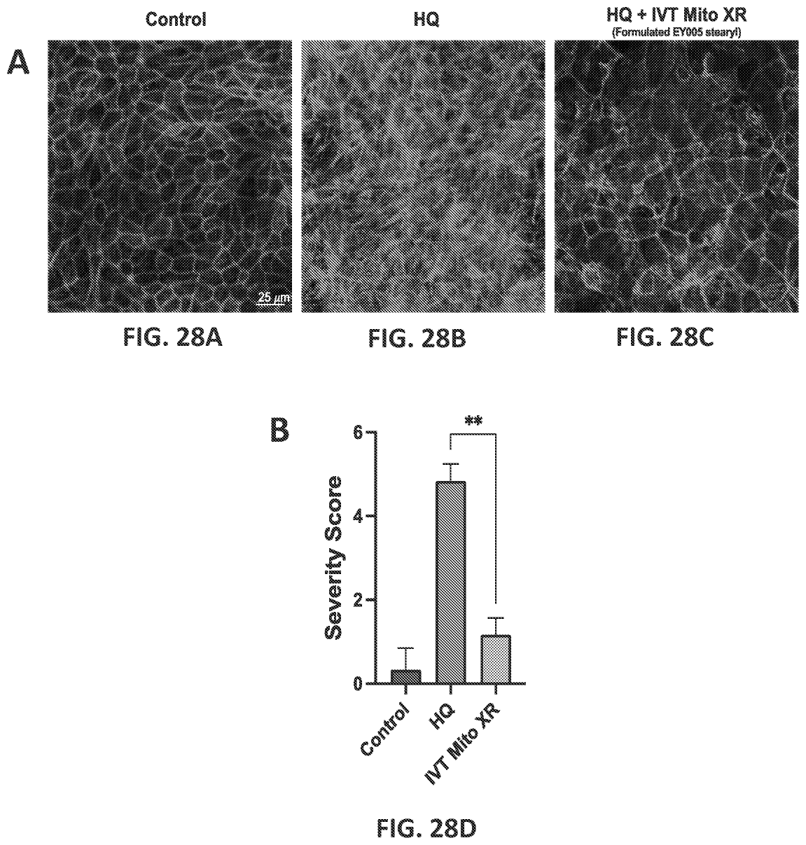

In in vitro efficacy studies, bolus implant of Mito XR was added to RPE cell culture model with endogenous esterases. Cell culture data demonstrated restoration of cytoskeleton, with ˜80% improvement at 21-day timepoint (FIGS. 28A-28D) in association with reversal of cellular mitochondrial dysfunction. This data affirms that EY005-stearyl, admixed with complexation agents and incorporated into a dispersal medium to form a stable multiphasic colloidal suspension in a formulation of Mito XR, can produce sustained release of EY005 at predictable therapeutic levels, which is bioactive upon cleavage of the MTT-prodrug that is released from the dispersal medium of the multiphasic colloidal suspension into the surrounding ocular physiologic environment.

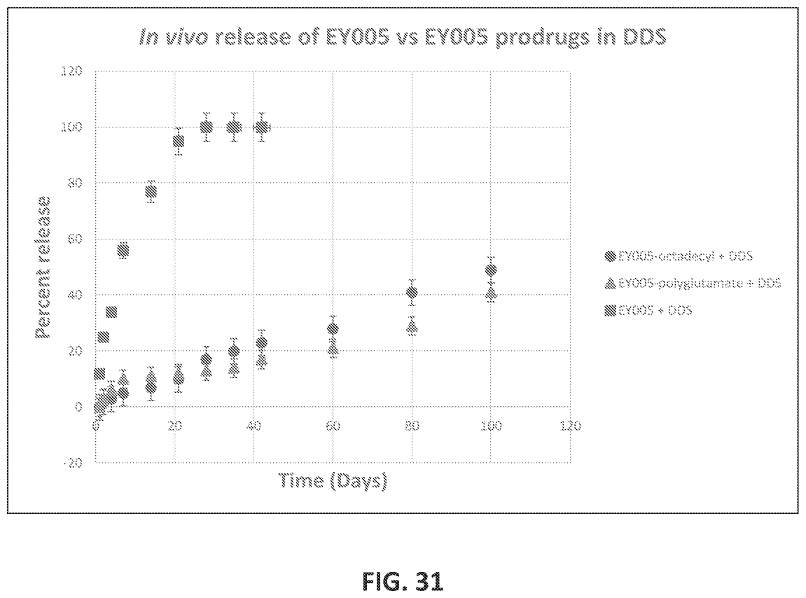

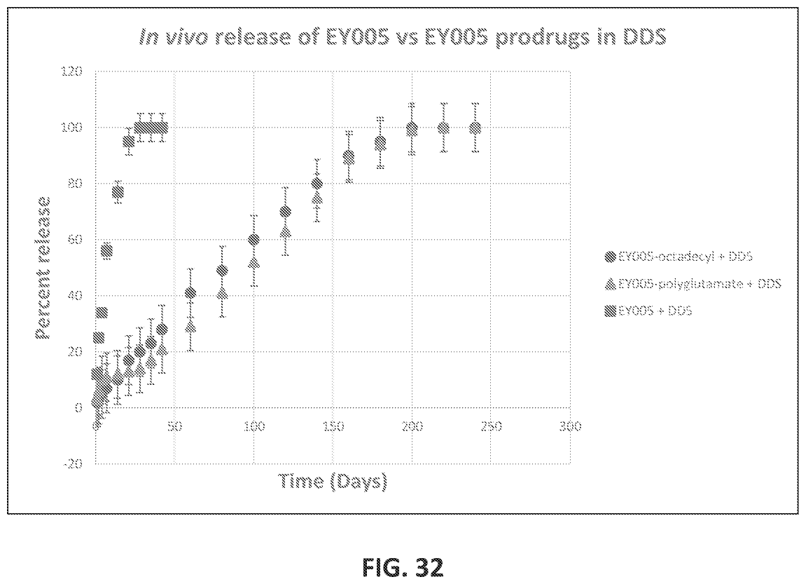

In some examples, the MTT-prodrug may be formulated within the presently described complexation-based extended release drug delivery system, Mito XR, deployed into the eye of animals or humans. For example, intravitreal administration of MTT-prodrug formulated within the complexation-based extended release drug delivery system in the eyes of rabbits has been found to produce sustained release of active MTT at the desired daily release rate and achieving desired target tissue levels of drug in the vitreous and retina (FIGS. 31-32).

In in vivo kinetics studies, using LC/MS analysis, we measured high retina EY005 levels (>300 ng/g) sustained through 6 weeks after IVT Mito XR (EY005-stearyl payload 1 mg) bolus injection in rabbit eyes (FIG. 29), affirming that endogenous esterases release active EY005 in vivo. Recovered bolus had ˜50% residual payload, indicating that implant formulation will achieve ˜90 day release of EY005 levels >EC₅₀, given zero-order release kinetics.

Importantly, formulation of Mito XR appeared to be well tolerated clinically in rabbit eyes (FIG. 30A), with no histologic evidence of toxicity (FIG. 30B).

In contrast to EY005-stearyl prodrug, the EY005 native tetrapeptide fails to form noncovalent interaction with complexation agent. FITC-labeled EY005 when admixed with different complexation agents (e.g., magnesium stearate, albumin), did not produce visible drug-complex particulates (FIGS. 20B, 21B, 22B and 23B).

Further, incorporation of EY005 native bioactive peptide with the same complexation agent and into the same dispersal medium used for Mito XR formulation of EY005-stearyl produced excessive release, or “dump” of the bioactive MTT in vitro (FIG. 27). Additionally, multiphasic colloidal suspension bolus formulation of EY005 native peptide administered into the vitreous did not produce detectable EY005 tissue levels beyond 21 days (FIG. 29), indicating excessive release of the native MTT drug in vivo as well. Moreover, no residual drug in the recovered bolus, consistent with excessive drug release or “dumping.” Thus, the incorporation of the native unmodified MTT into the multiphasic colloidal suspension is insufficient to produce sustained release and fails to achieve specifications of an extended release drug delivery system (see also FIGS. 31-32). Importantly, these data affirm and underscore the necessity for the prodrug construct and the specific interaction between prodrug conjugation moiety and complexation agent to form MTT-prodrug-complex particulates, to order to achieve controlled, durable release of the active MTT into the tissue following cleavage of covalent bond of the free MTT-prodrug released from the dispersal medium of multiphasic colloidal suspension.

The complexation agent may be noncovalently complexed with the conjugation moiety of the prodrug and incorporated and dispersed within a dispersal medium, comprising a formulation of complexation-based extended release drug delivery system, limiting diffusion of drug and restricting access of water from surrounding tissue to the particulates within the extended release drug delivery system (FIG. 25B).

The dispersal medium serves to stably disperse MTT-prodrug-complex particulates to form a stable multiphasic colloidal suspension. The dispersal medium may comprise one or more of: oils, liquid lipids, and semi-solid lipids. The dispersal medium may comprise one or more of: saturated fatty acid (methyl) esters, saturated fatty acid (ethyl) esters, unsaturated fatty acid (methyl) esters, and unsaturated fatty acid (ethyl) esters.

The MTT-prodrug compounds described herein, interacting with one or more particulate complexation agents to form MTT-prodrug-complex particulates, which, when admixed in the appropriate dispersal medium to form a stable multiphasic colloidal suspension and a resultant formulation of Mito XR, may provide a vitreous and retina concentration of the active MTT that meets or exceeds the EC₅₀ (i.e., effective concentration of the drug that produces 50% maximal response for reversal of mitochondrial dysfunction), for 1 to 12 months or more duration following a single administration of the Mito XR implant.

Sustained, high ocular tissue levels, and the resultant benefits for treatment of retina disease pathobiology described herein, are not feasible with systemic administration or with intravitreal administration of the unmodified mitochondrial-targeted peptide. Successful incorporation of MTT-prodrug into a compatible XRDDS, in this case, the complexation-based XRDDS is essential to achieve these therapeutic benefits for ocular and retinal diseases such as AMD.

For example, maximal systemic dosing of elamipretide is 40 mg daily delivered by subcutaneous route of administration, or approximately 0.4-0.9 mg/kg. In rabbits, following subcutaneous administration of 1 mg/kg elamipretide, peak choroid levels at 1 hour are ˜50 nM, which drops to undetectable after 4 hours; no drug is detected in the retina after subcutaneous administration in rabbit, due to the blood-retina barrier that impedes retinal penetration. Meanwhile, peak retina levels following intravitreal injection of 15 μg elamipretide is ˜15 μM. Thus, retinal bioavailability of mitochondrial-targeted peptides following systemic administration of a maximally tolerated dose is suboptimal as compared to intravitreal administration. Dosage level and frequency of dosing by subcutaneous route of administration is limited by toxicity of local injection site reactions that occur with high dose formulations or more frequent than daily dosing of mitochondria-targeted tetrapeptide drugs. Furthermore, since the half-life of the unmodified mitochondrial-targeted tetrapeptide in the vitreous is just 5-6 hours, intravitreal injection of the unmodified peptide drug does not produce durable tissue levels. The drug clears from the eye in just a few days, and once a week intravitreal injection in humans is not practical or feasible for treatment of disease in clinical practice. Thus, an extended release drug delivery system is essential to achieve sustained, high levels of drug within the vitreous, retina, and RPE.

In some examples, the composition, formulations and methods described herein, including prodrugs and the formulations of prodrugs incorporated into the complexation-based extended release drug delivery system (i.e., Mito XR), demonstrate a nonobvious and otherwise unexpected positive benefit on RPE dysmorphology, when administered at sufficiently high doses. In vitro and in vivo models of dry AMD, including the HQ exposure models in the protocols described in FIGS. 5 and 12, demonstrate additional pathologic features of dry AMD, including dysregulated extracellular matrix in the form of upregulated expression of cytosolic vimentin (intermediate filament that is upregulated in RPE of AMD eyes and secreted into the extracellular matrix) and RPE cell dysmorphology, in form of RPE cell actin cytoskeleton disorganization (FIGS. 7 and 14). Treatment with mitochondria-targeted tetrapeptide at sufficiently high drug levels, in this example EY005 (at 5 μM), reverses dysregulation of RPE extracellular matrix, downregulating expression of cytosolic vimentin, and restores normal RPE cell morphology, reversing actin cytoskeleton disorganization and clearing cytosolic actin aggregate formation (FIGS. 7, 9-10, 14).

Treatment with a sufficiently high dose of mitochondrial-targeted tetrapeptide, which is enabled by the composition, formulations and methods described herein in humans, also demonstrates a nonobvious and otherwise unexpected positive benefit on subRPE deposit formation. For example, FIG. 2A illustrates the reversal of pre-existing RPE mitochondrial dysfunction and sub-RPE deposits in an ApoE4 mouse model by high-dose elamipretide administered systemically by subcutaneous (SQ) administration (3 mg/kg BID). Aged ApoE4 mice fed a high-fat diet (HFD) develop sub-RPE deposits that reflect the pathobiology of subRPE drusen in patients with dry AMD. ApoE4 mice were treated with elamipretide (n=5; 3 mg/kg SQ daily) or vehicle (n=5) for 4 weeks, continuing HFD from prior 3 mos. FIG. 2A shows an analysis of outer retina morphology by transmission electron microscopy (TEM) in mice receiving vehicle 4 mos. after initiation of HFD, shown on the left. These untreated mice had thick, severe deposits. In contrast, mice receiving daily high-dose elamipretide by SQ administration for 4 weeks demonstrated minimal deposits and restoration of outer RPE cellular morphology, as shown on the right panel of FIG. 2A.

Sufficiently high doses can be achieved in rodents by systemic SQ administration due to their small size and due to the minimal amount of drug dose and volume required for dosing. This is not possible in humans due to larger size of humans and due to dose-limiting toxicities of local injection site reactions with SQ formulations of elamipretide; maximally tolerated dosing for human is 40 mg SQ once daily, approximately 0.3-0.9 mg/kg, or approximately 3-10 fold lower dosing than efficacious dosing in mice (FIG. 3). Taken together, these data suggest that treatment with sufficiently high doses of mitochondrial-targeted drugs, including the presently described compositions, formulations, and methods relating to prodrugs and intravitreally administered Mito XR described herein, which utilize analogs of elamipretide specifically formulated to achieve sufficiently high dose to the eye and sufficiently high ocular tissue drug levels, can promote regression of subRPE deposits and restoration of RPE cellular morphology and health.

Systemic delivery of mitochondrial targeted tetrapeptides by SQ injection have suboptimal efficacy in human patients due to insufficient retinal bioavailability. Sufficient levels of the mitochondrial-targeted tetrapeptides cannot be achieved at the target tissue site (i.e., retina). For example, in humans, maximal dosing is 40 mg SQ (˜0.3-0.9 mg/kg), a dose that is limited by SQ injection site toxicity (FIG. 3). In mice, effective dosing required 3 mg/kg, which is 3-10 fold greater than the mass-adjusted dose in humans. Peak plasma levels are also limited by the drug's rapid blood clearance, and the blood-eye barrier may also impede retinal penetration. Accordingly, at 1-8 hrs. after SQ administration of 1 mg/kg elamipretide in rabbits, peak choroid levels at 1 hr. are ˜50 nM, which falls to undetectable after 4 hrs.; no drug is detected in the retina, indicating that the SQ route of administration is also inadequate for achieving necessary tissue levels at sufficient duration. In vitro EC₅₀ for prevention of mitochondrial dysfunction is ˜10-100 nM and is ˜1 μM for reversal (48 hrs. duration of drug exposure). Thus, higher retinal and choroidal drug levels with continuous drug exposure are desirable. The MTT-prodrugs and the formulations of Mito XR (prodrugs formulated in multiphasic colloidal suspension) described herein enable IVT or periocular administration over longer periods of time than are otherwise achievable by systemic administration.

In some examples, the composition, formulations and methods described herein, including prodrugs and the formulations of prodrugs incorporated into the complexation-based extended release drug delivery system (i.e., Mito XR), demonstrate reversal of mitochondrial dysfunction. For example, in the rabbit ocular hydroquinone model, systemic administration of the maximally tolerated human dose of mitochondrial-targeted peptide (EY005) allometrically scaled for the rabbit, only partially restores RPE actin cytoskeletal integrity (FIG. 15D). In contrast, IVT injection of EY005 results in downregulation of RPE vimentin expression (FIG. 14) and restoration of normal RPE actin cytoskeleton integrity (FIG. 15C). In addition, FIG. 13 shows the results for a single IVT dose of 15 μg of EY005 or vehicle, at 36 hrs. after drug treatment. In FIG. 13, the far right column shows normal appearance for each of DCFDA (oxidation products marker). The left column shows untreated, HQ-exposed eyes and the middle column shows the EY005-treated, HQ-exposed eyes. IVT EY005 treatment produced an approximately 75% reduction of oxidation byproducts. The results indicate that a single dose of IVT EY005 reverses both existing severe mitochondrial dysfunction in vivo within 36 hrs. or less, when administered at sufficiently high dose. Importantly, no retinal toxicity was observed. In this setting, retinal and RPE tissue levels achieved by IVT administration in rabbits are substantially higher than tissue levels obtained by SQ administration of the maximally tolerated human dose allometrically scaled for rabbits.

In some examples, the composition, formulations and methods described herein, including prodrugs and the formulations of prodrugs incorporated into the complexation-based extended release drug delivery system (i.e., Mito XR), demonstrate a nonobvious and otherwise unexpected positive benefit on RPE dysmorphology, when administered at sufficiently high doses by intravitreal (IVT) route. FIG. 14 shows the results for a single IVT dose of 15 μg of EY005 or vehicle, at 36 hrs. after drug treatment. As evident by Vimentin (marker of extracellular matrix dysregulation) and Phalloidin (label for actin cytoskeleton and cell morphology) staining, IVT EY005 treatment produced an approximately 90% reversal of vimentin stain, and approximately 80% reduction of cytoskeleton disorganization, demonstrating reversal of RPE cell injury and dysmorphology and restoration of RPE health.

In some examples, IVT Mito XR formulation of prodrug produces sufficiently high ocular tissue levels of drug for a sustained period of time with continuous drug release for 3 months or greater (FIG. 29), resulting in disease modification with not only mitochondrial dysfunction but also nonobvious and otherwise unexpected positive effects on RPE morphology and health and retinal visual function, in the setting of dry AMD and potentially other retinal and posterior segment diseases.

The compositions and methods described herein, including Mito XR, may be applied by delivery of the implant to the eye by intravitreal or periocular routes of administration to treat various retinal and back of the eye diseases, include dry age-related macular degeneration (AMD), wet AMD, diabetic retinopathy (DR), retinal vein occlusion (RVO), acquired and inherited retinal degenerations, and other retinal and optic nerve diseases.

The composition, formulations and methods described herein may be used to treat dry AMD, in a patient in need thereof, including a patient already having dry AMD, a patient at risk for dry AMD, a patient at risk for progression of dry AMD, or a patient at risk for vision loss as a result of advanced forms of AMD (e.g., geographic atrophy (GA), either central or noncentral GA).

The composition, formulations and methods described herein may be used to treat neovascular AMD, in a patient in need thereof, including a patient already having neovascular AMD, a patient at risk for neovascular AMD, a patient at risk for progression of neovascular AMD, or a patient at risk for vision loss as a result of advanced forms of neovascular AMD (e.g., atrophic disease, fibrosis, scar, etc.).

The composition, formulations and methods described herein may be used to treat and prevent progression from dry to neovascular AMD, in a patient in need thereof, a patient at risk for neovascular AMD, or a patient at risk for vision loss as a result of progression from dry to neovascular AMD.

The composition, formulations and methods described herein may be used to treat retinal vein occlusion (RVO), in a patient in need thereof, including a patient already having RVO, a patient at risk for RVO, a patient at risk for progression of RVO, or a patient at risk for vision loss as a result of advanced forms of RVO (e.g., macular edema, retinal nonperfusion, retinal hemorrhage, retinal ischemia, retinal or ocular neovascularization, retinal atrophy, vitreous hemorrhage, etc.).

Implants of Mito XR have been characterized by in vitro studies for release and cellular efficacy and by in vivo studies for toxicology, pharmacokinetics (PK), and efficacy, demonstrating their potential utility for clinical use in humans and animals affected by retinal diseases.

The release of the bioactive drug from the implant is dependent on the diffusion of free, unbound MTT-prodrug within the dispersal medium of the multiphasic colloidal suspension into the surrounding ocular physiologic environment and release of the active MTT from the prodrug by cleavage of the covalent bond either via natural enzymes within the tissue compartment of the body (i.e., within the vitreous or within periocular tissues). Alternatively, release of the active MTT from the prodrug may occur by hydrolysis of MTT-prodrug that is released from the implant into the ocular physiologic environment.

A therapeutic composition for local ocular administration may include: any of the MTT-prodrugs described herein, where the conjugation moiety of the MTT-prodrug forms noncovalent interaction (complex) with selected compatible complexation agent to form MTT-prodrug-complex particulates, which are then incorporated and admixed within a hydrophobic dispersal medium to form a stable multiphasic colloidal suspension. The combined effect of conjugation moiety, complexation, and stable dispersion of complex particulates within multiphasic colloidal suspension alters the physicochemical properties of the active MTT drug, limits the amount of free MTT-prodrug available for release from the implant into the ocular physiologic environment, and restricts access of water to free drug and MTT-prodrug-complex particulates, facilitating sustained release and continuous, predictable exposure of therapeutic levels of active drug for desired duration of disease treatment.

Also described herein methods of treating mitochondrial dysfunction in and around the eye by using an MTT-prodrug comprising a bioactive MTT that is covalently linked to an inactive conjugation moiety that facilitates noncovalent interactions between the conjugation moiety of the prodrug and a complexation agent within a dispersal medium and serves to limit the amount of free MTT-prodrug within the dispersal medium.

In general, a method of treating mitochondrial dysfunction in or around the eye may include administering any of the therapeutic compositions described herein.

Also described are methods of treating or preserving neurosensory retina structure including ellipsoid zone, treating RPE dysmorphology, RPE-associated extracellular matrix dysregulation, abnormal RPE metabolism, sub-RPE deposit, and/or drusen deposits, by administering any of the therapeutic compositions described herein to specifically enable intravitreal or periocular injections of formulations of Mito XR that produce sufficiently high sustained retina and RPE tissue levels of active drug to modify these pathologic features of disease.

Also described are methods of improving vision or preventing vision loss in patients with retinal and ocular diseases, by administering any of the therapeutic compositions described herein to specifically enable intravitreal or periocular injections of formulations of Mito XR that produce sufficiently high sustained ocular tissue levels of active drug to improve function of relevant ocular tissues.

Also described are methods of preventing onset or progression of atrophic retinal disease, e.g., geographic atrophy, by administering any of the therapeutic compositions described herein to specifically enable intravitreal or periocular injections of formulations of Mito XR that produce sufficiently high sustained retina and RPE tissue levels of active drug to restore cellular health, limit cell death, and prevent progressive loss of vital tissue.

In any of these methods the active MTT may be released via cleavage of prodrug by esterases present within the vitreous or other tissues of the eye. The active mitochondria targeted peptide may be released via hydrolysis or other reaction that results in release of the bioactive mitochondrial-targeted peptide drug. The released bioactive MTT drug may be H-d-Arg-DMT-Lys-Phe-OH, or any MTT disclosed in the list in Table 1 (SEQ ID NOs. 1-635).

Administration may comprise local ocular administration via injection of an implant of Mito XR.

Mito XR may be administered into the eye using intravitreal (IVT), periocular, sub-Tenon's, subconjunctival, or intracameral routes. The administration may comprise injecting a formulation of Mito XR as a modality of bolus into the vitreous of the eye.

Administration may comprise injecting a formulation of prodrug within the Mito XR implant (multiphasic colloidal suspension) as a modality of a sustained release drug formulation device. The extended release drug delivery system may comprise delivering a bioerodible or non-bioerodible implant into a vitreous of the eye.

Any of these methods may include treatment intervals of 1-12 months for administering Mito XR into the subject's eye. The method may be a method of treating retinal and optic nerve diseases, including dry age-related macular degeneration (AMD), wet AMD, diabetic retinopathy (DR), retinal vein occlusion (RVO), retinitis pigmentosa (RP), glaucoma, optic nerve disease, or for neuroprotection of the retina and/or optic nerve.

The method may be used in conjunction with other treatment modalities including inhibition of vascular endothelial growth factor, complement inhibition, or administration of anti-inflammatory drugs such as corticosteroids.

A method of treatment of mitochondrial dysfunction in a subject's eye may include delivering a MTT-prodrug incorporated into formulations of Mito XR into the subject's eye at a treatment start; and cleavage of the covalent bond of the prodrug to release the active MTT into the eye during a first phase at a burst phase release rate; subsequently during a second phase at a steady-state release rate, wherein the burst phase rate is greater than the steady state dose rate, further wherein the first phase extends from the treatment start for about 2-6 weeks and the subsequent phases (second phase, and in some instances second and third phases) extend from an end of the first phase for one or more months.

A method of treatment of RPE dysmorphology or sub-RPE deposits in a subject's eye, may include delivering a MTT-prodrug incorporated into formulations of Mito XR into the subject's eye at a treatment start; and cleavage of the covalent bond of the prodrug to release the active MTT into the eye during a first phase at a burst phase release rate; subsequently during a second phase at a steady-state release rate, wherein the burst phase rate is greater than the steady state dose rate, further wherein the first phase extends from the treatment start for about 2-6 weeks and the subsequent phases (second phase, and in some instances second and third phases) extend from an end of the first phase for one or more months.

A method of treatment of vision loss in a subject may include delivering a MTT-prodrug incorporated into formulations of Mito XR into the subject's eye at a treatment start; and cleavage of the covalent bond of the prodrug to release the active MTT into the eye during a first phase at a burst phase release rate; subsequently during a second phase at a steady-state release rate, wherein the burst phase rate is greater than the steady state dose rate, further wherein the first phase extends from the treatment start for about 2-6 weeks and the subsequent phases (second phase, and in some instances second and third phases) extend from an end of the first phase for one or more months.

A method of preventing onset or progression of atrophic retinal disease in a subject may include delivering a MTT-prodrug incorporated into formulations of Mito XR into the subject's eye at a treatment start; and cleavage of the covalent bond of the prodrug to release the active MTT into the eye during a first phase at a burst phase release rate; subsequently during a second phase at a steady-state release rate, wherein the burst phase rate is greater than the steady state dose rate, further wherein the first phase extends from the treatment start for about 2-6 weeks and the subsequent phases (second phase, and in some instances second and third phases) extend from an end of the first phase for one or more months.

Another embodiment of an XRDDS includes MTTs (including elamipretide), MTT-prodrug, and MTT-prodrug-complex particulates formulated within a retention vehicle. A retention vehicle is a liquid or semi-solid substance in which the vehicle is chosen based on its physicochemical properties for interaction with drug substance in a manner that restricts or limits its release from the retention vehicle. Examples include but are not limited to oil-in-water emulsions, water-in-oil emulsions, viscous gelatin, hydrogels, and viscous chondroitin sulfate, all of which can be used to formulate MTT (including elamipretide) MTT-prodrug, and MTT-prodrug-complex particulates. A retention vehicle-based XRDDS does not have any requirement for stable dispersal of MTT-prodrug or MTT-prodrug-complex particulates, and drug release is determined by the interaction of the retention vehicle with the drug substance, wherein the retention vehicle impedes or slows diffusion from the vehicle into the ocular physiologic environment. These properties differ from the example of MTT-prodrug in the multiphasic colloidal suspension XRDDS, wherein the MTT-prodrug-complex particulates are stably dispersed without settling or migration, and there is no requirement that the dispersal medium impedes or slows diffusion of the MTT-prodrug from the implant.

Another embodiment of an XRDDS includes MTTs (including elamipretide), MTT-prodrug, and MTT-prodrug-complex particulates formulated within a carrier-based XRDDS, a passive-release, bio-erodible formulation strategy. Carrier-based XRDDS are designed to physically trapped in a specific carrier, but then the system must degrade via interactions with the tissue, not from mechanisms intrinsic within the XRDDS, in order to release free drug. In some embodiments, carrier formulations include a single device that compartmentalizes drug from the tissue. Examples include but are not limited to polymer-based rods or other shapes (drug trapped in a chemical substance extruded into rods or molded into different shapes), photopolymerizable or photo-crosslinked block polymer comprising PLGA and other cross-linkable substrates in which drug is trapped within the polymer formulated into injectable viscous polymer or polymer-based rods or other shapes, polymer-based microparticles (which require chemical covalent crosslinking of small block polymers to trap drug), liposomes (phospholipid-in-water emulsion) sonicated to trap drug, all of which can be used to formulate MTT (including elamipretide) MTT-prodrug, and MTT-prodrug-complex particulates. The common feature of all carrier-based systems is that the drug is trapped within the carrier material; as the carrier degrades, dissolves, or otherwise breaks down, free drug is released into the tissue. This may require a chemical or enzymatic reaction provided by the tissue microenvironment. In addition, the defects made in the carrier system during degradation allow access to water from the microenvironment, which further promotes release of the drug substance. Carrier-based systems differ from the multiphasic colloidal suspension, which has a hydrophobic dispersal medium and therefore repels water from entering the system. Further, in the multiphasic colloidal suspension, there is no requirement for complexation-based XRDDS to degrade via interactions with the tissue in order to release MTT-prodrug from the implant. The release kinetics are not determined by drug trapped complexation-based XRDDS. Free drug is present in the dispersal medium, and the release kinetics for the multiphasic colloidal suspension are determined by diffusion of drug out of an intact system, which is hydrophobic and water repellent.

Described herein are methods of manufacturing for Mito XR, wherein a selected MTT-prodrug is admixed with a complexation agent particulate to form MTT-prodrug-complex particulate. One or more MTT-prodrug-complex particulate(s) are then added and incorporated to a selected dispersal medium to form the stable multiphasic colloidal suspension. The resultant formulation of MTT-prodrug, complexation agents, and dispersal medium forms the implant of Mito XR.

The property of Kd is a measure of avidity of an MTT-prodrug for a given complexation agent and is defined as the unbound-bound fraction of MTT-prodrug for an MTT-prodrug-complex particulate in a given dispersal medium. Specific Kd value can be measured by specified release assay, as described herein.

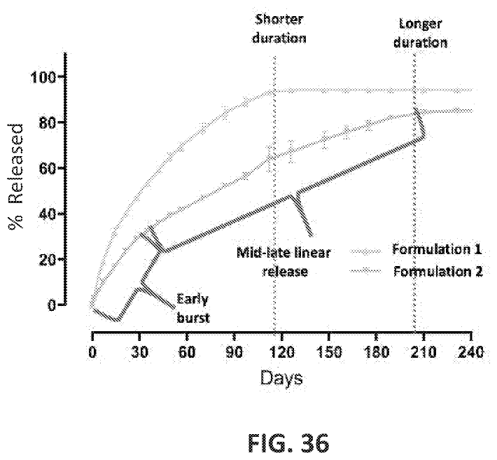

The regulation of release of MTT-prodrug from the implant is determined by the unbound fraction within the dispersal medium, which is in turn determined by the Kd, defined as the ratio of unbound to bound MTT-prodrug for a given complexation agent within a specific dispersal medium. Knowledge of the Kd for a particular MTT-prodrug-complex particulate allows the choice of specific combinations of prodrug-complexation agent to achieve a prespecified release kinetics profile. The inclusion of more than one complexation agent in the multiphasic colloidal suspension can be used to regulate the unbound fraction of drug within the dispersal medium over time and thus the release kinetics of the system (see FIGS. 35A-35E and 36).

For example, in some formulations of Mito XR, there may be a first phase and a second phase of release, wherein there is increased release of the mitochondrial targeted tetrapeptide during the first phase, and a subsequent lower release of mitochondrial targeted tetrapeptide during the second phase (see FIG. 36). This formulation may be achieved by the combination of two different MTT-prodrug-complex particulates, wherein one complex particulate has high Kd, reflecting low affinity of MTT-prodrug for first complexation agent) and the second complex particulate has low Kd, reflecting high affinity of MTT-prodrug for second complexation agent. In this setting, the first phase of release may be a “burst” faster rate of MTT-prodrug release from the higher Kd (low affinity) particulate, and the second phase of release is a slower, steady-state of MTT-prodrug release from the lower Kd (higher affinity) particulate. In this manner, different MTT-prodrug-complex particulates can be specifically selected and combined, in desired ratio and proportion, to achieve a prespecified kinetic profile of MTT-prodrug release from Mito XR formulation.

In such examples, the combined effect for a combination of two or more MTT-prodrug-complex particulates incorporated into selected dispersal medium is release of the MTT in two or more phases based on the integral of release rates from the individual drug-complexation agent particulate components that are incorporated and dispersed within the Mito XR implant.

The actual release kinetics of achieved by Mito XR in in vivo vitreous concentrations may meet or exceed EC₅₀ for an extended-release duration of 1 month or more. The EC₅₀ reflects the concentration of the MTT-prodrug compound that achieves 50% of the maximal response for reduction in mitochondrial dysfunction measured both for reversal of pre-existing mitochondrial dysfunction and for prevention of new onset mitochondrial dysfunction, by specific readouts of mitochondrial dysfunction.

In the presently described extended release drug delivery system, specific formulations achieving a desired target release profile for a given release duration and total payload can be custom designed by mathematical formula and subsequently constructed by iterative refinement. Two or more sets of “drug-complex” particulates with distinct Kd values can be combined in different ratios and amounts to specifically design, customize, and “tune” a target drug kinetic release profile (i.e., daily drug release rate) using a mathematical formula that takes into account the individual Kd values and integrates the drug release rates of the individual sets of drug-complex particulates when combined in dispersal medium. The target release profile can be designed with one or more phases of release kinetics, for a given drug payload and a desired duration of drug release.

For example, FIGS. 35A-35E illustrates the theoretical basis for design and construction of an extended release drug delivery system (XRDDS) implant producing a desired drug release kinetic profile for bioactive mitochondrial targeted drug. Initially, a theoretical pharmacokinetic release curve (i.e., target release profile), in this depiction linearized by log transformation (FIG. 35A), is designed representing the desired initial burst phase and subsequent steady-state release phase, to give desired daily release rate, total duration of delivery, and drug payload in the final extended release drug delivery system implant. An iterative process is the performed to identify specific member compounds from 2 or 3 difference classes of complexation agents, expected to form noncovalent interactions with the particular conjugation moiety of the prodrug based on the physicochemical properties of the prodrug and its conjugation moiety. Each drug-complexation agent is first combined at initial amount and ratio and drug-complex particulates are then admixed and incorporated within a proposed dispersal medium. The drug-complex-medium system is put into “sink” conditions and two properties of the drug-complex pair are measured: the Kd (unbound-bound fraction) at day 1, 3, 7, 14, and 21 (a good indicator of burst and general binding avidity); and the release kinetics (% of initial payload of drug released over time), where Kd1 corresponds to drug-complex 1 and Kd2 corresponds to drug-complex 2 (FIGS. 35C1 and 35C2, respectively).

Curve fitting is then applied to the release curve of each drug-complex, and the linearized curves are then solved to determine the right combination (of 2 or 3 specific drug-complex pairs) that give release kinetics that meet the pre-determined desired composite target product profile (FIG. 35D).

As shown in FIG. 35D, this “theoretically-designed” formulation containing the combination of 2 or 3 drug-complex pairs are then formulated and tested for actual release kinetics. If necessary, the ratios of the 2-3 selected drug-complex pairs can be re-adjusted iteratively until the final release kinetics meet the predetermined target product release profile (FIG. 35E).

In some instances, for the second or third drug-complex pair, the bioactive drug may be covalently linked to a different conjugation moiety to form a different prodrug structure and the complexation agent may be distinct from the first, with distinct Kd values, Kd1 and Kd2 of drug-complex pairs, based both on the differing conjugation moieties and the differing complexation agent between pairs.

Alternatively in some instances, the conjugation moiety of the prodrug may differ between the first and second drug-complex pairs, but the complexation agent may be the same, with distinct Kd values, Kd1 and Kd2 of drug-complex pairs, based on the differing conjugation moieties between pairs.

The composite extended release drug delivery system is designed and customized for the physicochemical properties of the MTT-prodrug to regulate the release of free MTT-prodrug from the system into the tissue, where the prodrug is cleaved by esterases or by hydrolysis to release the active MTT.

In formulations of Mito XR with two-phase release kinetics, the concentration of MTT-prodrug in the vitreous may exceed the reversal EC₅₀ during the initial burst phase and subsequently exceed the prevention EC₅₀ for the second (steady-state) phase, and release kinetics, selection of specific MTT-prodrug-complex particulates, specific ratio and concentration of different particulate combinations, and total payload of MTT-prodrug in the Mito XR formulation may be selected to achieve this designed release kinetics for desired duration of drug release. Two-phase release kinetics may be desirable for an “loading dose” phase of drug release to reverse pre-existing disease manifestations and a subsequent “maintenance” phase of drug release to prevent the recurrence of disease manifestations.

In formulations of Mito XR with single-phase release kinetics, the concentration of MTT-prodrug in the vitreous may exceed the EC₅₀ for reversal of mitochondrial dysfunction.

In formulations of Mito XR with three-phase release kinetics, the concentration of MTT-prodrug in the vitreous may exceed the EC₅₀ for reversal of mitochondrial dysfunction during the first phase, may exceed the EC₅₀ for prevention of mitochondrial dysfunction for steady-state release during the second phase, and may exceed EC₅₀ for reversal of mitochondrial dysfunction during the third, late-burst phase. Three-phase release kinetics with third phase of late “burst” may be desirable for settings in which there is loss of potency of drug due to tachyphylaxis or due to increased triggers or drivers of cellular mitochondrial dysfunction and/or retinal or RPE disease.

For example, FIG. 36 illustrates release kinetics for examples of two distinct formulations of EY005 prodrugs in the complexation-based XRDDS, each with different conjugation moieties, as described herein: EY005-octadecyl (formulation 1) and EY005-8-mer peptide (formulation 2). As shown in FIG. 36 both formulation 1 and formulation 2 had two-phase kinetics, with an early burst followed by a more linear release. However, formulation 1 had a longer initial early burst, resulting in 120-day durability, while formulation 2 had a shorter early burst and a longer steady-state release phase, resulting in 210 days of release.

The relation Cₛₛ=Release rate/Clearance and the half-life (t_{1/2}) can be utilized to calculate the approximate desired daily release rate and drug payload of the extended release drug delivery system implant.

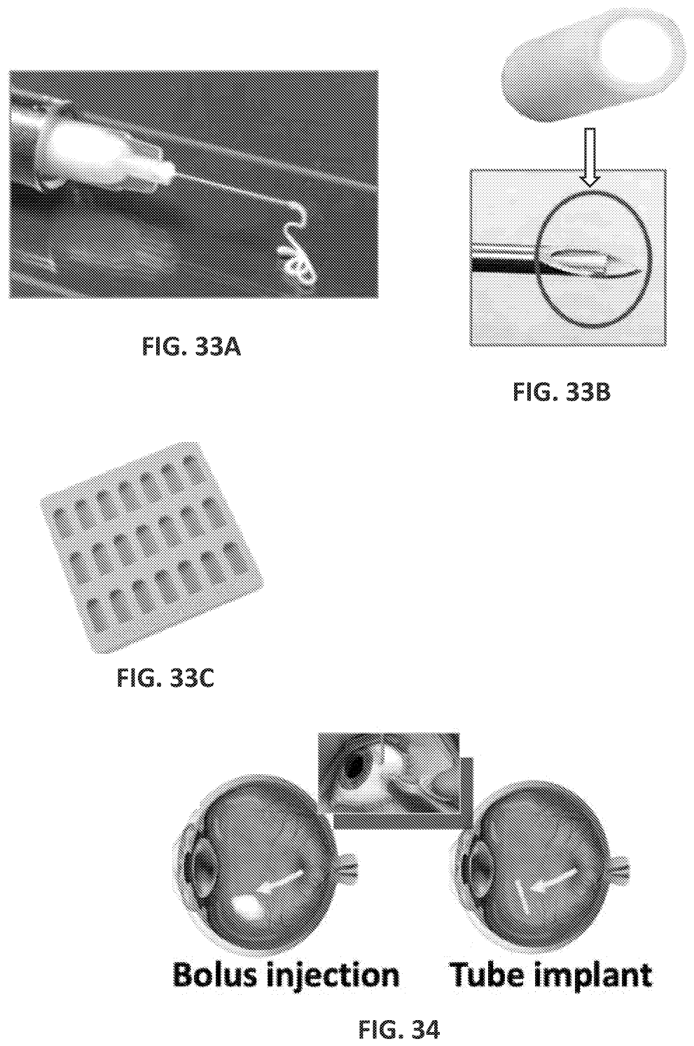

As an example, a payload of approximately 100 μg of drug may achieve 6-8 months of efficacy and durability with a release rate of ˜200-500 ng per day. A 2-phase release kinetic may include an early burst (to load the retina with drug) for 1 month followed by 5-7 months of steady-state release. Complexation agents that are expected to interact favorably with the conjugation moiety of the selected prodrug to limit diffusion of the prodrug compound would then be selected and incorporated into a tube implant or bolus modality of the extended release drug delivery system (e.g., see FIGS. 33A and 33B).

The multiphasic colloidal suspension may be formulated as one of several modalities of the complexation-based extended-release drug delivery system that may be injected into the vitreous (FIGS. 33 and 34), including a flowable bolus implant (FIG. 33A), a solid mold of a specific size and shape, or a semi-solid that fills a bioerodible or non-bioerodible sleeve or outer covering to form a tube implant (FIG. 33B). In some examples, the tube may itself be formed of the extended release drug delivery system. In other examples, the tube may be a comprised of a bio-erodible polymer that is compatible with ocular tissues (e.g., poly(lactic-co-glycolic acid) PLGA). In some examples, the tube may have one or both ends open for release of the MTT-prodrug. The tube may be injected via needle or cannula into the vitreous, as shown in FIG. 34 (right) or into periocular tissues. In some examples, the extended release drug delivery system incorporating MTT-prodrug may be molded into shapes (FIG. 33C)

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

The process parameters and sequence of steps described and/or illustrated herein are given by way of example only and can be varied as desired. For example, while the steps illustrated and/or described herein may be shown or discussed in a particular order, these steps do not necessarily need to be performed in the order illustrated or discussed. The various example methods described and/or illustrated herein may also omit one or more of the steps described or illustrated herein or include additional steps in addition to those disclosed.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1.-42. (canceled)

43. A prodrug compound of formula (I): 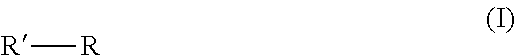
where R′ is a mitochondrial targeted tetrapeptide (MTT) containing alternating cationic and aromatic amino acid residues in which the C-terminal amino acid is covalently linked to R by a cleavable covalent bond, where R is a conjugation moiety that may be removed by enzymatic cleavage, catalysis, hydrolysis, or other reaction to yield free mitochondria targeted tetrapeptide R′ and conjugation moiety R, where R is selected from:
a C4-C30 lipid moiety (fatty acid or fatty alcohol),
an C4-C30 straight-chain or branched aliphatic moiety,
a 2-mer to 30-mer peptide moiety,
a pegylated moiety, or
a carbohydrate moiety.

44. The prodrug compound of claim 43, having the formula of formula (III): 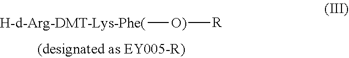 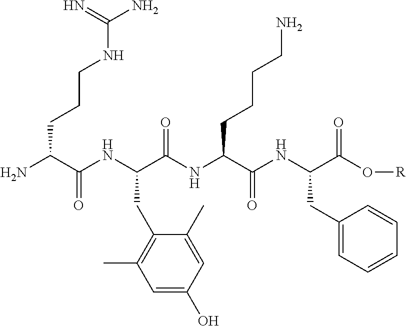
where the fourth amino acid is linked via ester bond to R, and R or —O—R is a conjugation moiety selected from:
a C4-C30 lipid moiety, (fatty acid or fatty alcohol),
an C4-C30 straight-chain or branched aliphatic moiety,
a 2-mer to 30-mer peptide moiety, or
a pegylated moiety, or
a carbohydrate moiety.

45. The prodrug compound of claim 43 having the formula: H-d-Arg-DMT-Lys-Phe(-O)-octadecyl; H-d-Arg-DMT-Lys-Phe(-O)-Arg₍ₙ₎, where n is between 1 and 30; H-d-Arg-DMT-Lys-Phe(-O)-Glu₍ₙ₎, where n is between 1 and 30.

46. The prodrug compound of claim 43, wherein the MTT is a mitochondria targeted peptide from any of SEQ ID NOs. 1-635.

47. The prodrug compound of claim 43, wherein the cleavable covalent bond comprises one of: an ester bond, a hydrazone bond, an imine bond, a disulfide bond, a thioester bond, a thioether bond, a phosphate ester bond, a phosphonate ester bond, a boronate ester bond, an amide bond, a carbamate ester bond, a carboxylate ester bond, and a carbonate ester bond.

48. The prodrug compound of claim 43, wherein the conjugation moiety is fatty alcohol, with or without a preceding linker moiety, that includes the following: tert-butyl alcohol, tert-amyl alcohol, 3-methyl-3-pentanol, 1-heptanol (enanthic alcohol), 1-octanol (capryl alcohol), 1-nonanol (pelargonic alcohol), 1-decanol (decyl alcohol, capric alcohol), undecyl alcohol (1-undecanol, undecanol, hendecanol), dodecanol (1-dodecanol, lauryl alcohol), tridecyl alcohol (1-tridecanol, tridecanol, isotridecanol), 1-tetradecanol (myristyl alcohol), pentadecyl alcohol (1-pentadecanol, pentadecanol), 1-hexadecanol (cetyl alcohol), cis-9-hexadecen-1-ol (palmitoleyl alcohol), heptadecyl alcohol (1-n-heptadecanol, heptadecanol), 1-octadecanol (stearyl alcohol), 1-octadecenol (oleyl alcohol), 1-nonadecanol (nonadecyl alcohol), 1-eicosanol (arachidyl alcohol), 1-heneicosanol (heneicosyl alcohol), 1-docosanol (behenyl alcohol), cis-13-docosen-1-ol (erucyl alcohol), 1-tetracosanol (lignoceryl alcohol), 1-pentacosanol, 1-hexacosanol (ceryl alcohol), 1-heptacosanol, 1-octacosanol (montanyl alcohol, cluytyl alcohol), 1-nonacosanol, 1-triacontanol (myricyl alcohol, melissyl alcohol).

49. The prodrug compound of claim 43, wherein the conjugation moiety is a fatty acid, with or without a preceding linker moiety, that includes the following: Tetradecanoic acid, pentadecanoic acid, (9Z)-hexadecenoic acid, Hexadecanoic acid, Heptadecanoic acid, Octadecanoic acid, (9Z,12Z)-octadeca-9,12-dienoic acid, (9Z,12Z,15Z)-octadeca-9,12,15-trienoic acid, (6Z,9Z,12Z)-octadeca-6,9,12-trienoic acid, (5E,9E,12E)-octadeca-5,9,12-trienoic acid, (6Z,9Z,12Z,15Z)-octadeca-6,9,12,15-tetraenoic acid, (Z)-octadec-9-enoic acid, (11E)-octadec-11-enoic acid, (E)-octadec-9-enoic acid, nonadecanoic acid, and eicosanoic acid.

50. The prodrug compound of claim 43, wherein R is a 2-mer to about a 30-mer peptide moiety comprising natural or synthetic amino acids, which may be anionic, cationic, or neutral, with or without a preceding linker moiety, that includes the following: poly-glutamate, poly-aspartate or a combination of glutamate and aspartate; poly-arginine, poly-lysine, poly-histidine, a combination of arginine and lysine, a combination of arginine and histidine, a combination of histidine and lysine, a combination of arginine, histidine, and lysine; peptide moiety has one or more PEGylation sites for addition of polyethylene glycol (PEG) groups; peptide moiety has one or more sites for modification by addition of sugar or carbohydrate molecules, including glycosylation; polyarginine moiety; polyglutamate moiety; polyaspartate moiety; polyhistidine moiety; polylysine moiety.

51. The prodrug compound of claim 43, wherein R is a polyethylene glycol (PEG) polymer, a pegylated peptide, or pegylated succinate including PEG polymers of linear, branched, Y-shaped, or multi-arm geometries.

52. The prodrug compound of claim 43, wherein R is a carbohydrate moiety comprising a carbohydrate of 2 to 20 sugars, with or without a preceding linker moiety, that includes the following; glucose, galactose, lactose, mannose, ribose, fucose, N-acetylgalactosamine, N-acetylglucosamine, N acetylneuraminic acid, or an epimer or derivative of glucose, galactose, lactose, mannose, ribose, fucose, N-acetylgalactosamine, N-acetylglucosamine, and N acetylneuraminic acid.

53. The prodrug compound of claim 43, wherein R′ is the MTT, including H-d-Arg-DMT-Lys-Phe, and R is a linker or multimerization domain which is convalently linked to multiple mitochondria targeting peptides to form dimers or multimers of the prodrug and n is equal to 2 to about 100, and R may be PEG, a PEG polymer, polyvinyl alcohol (PVA), or peptide.

54. A composition of a multiphasic colloidal suspension comprising a mitochondrial targeted tetrapeptide (MTT)-prodrug and one or more complexation agents, admixed in a dispersal medium.

55. The composition of claim 54, wherein the MTT is a mitochondria targeted peptide having sequence from one of SEQ ID NO 1-635.

56. The composition of claim 54, wherein the complexation agent is a chemical substance formulated as an irregular shaped particulate, capable of forming MTT-prodrug-complex particulates, selected from one of six classes: fatty acid, organic compounds that can form keto-enol tautomers, charged phospholipid, charged protein, ribonucleic acid, and polysaccharide.

57. The composition of claim 54, wherein the complexation agent is a fatty acid, which is a carboxylic acid with an aliphatic chain with chemical formula of CH3(CH2)ₙCOOH where n is equal to between 4 and 30, which may be either saturated or unsaturated and may be in the form of a salt or ester, and includes the following: magnesium palmitate, magnesium stearate, calcium palmitate, calcium stearate.

58. The composition of claim 54, wherein the complexation agent is one or more of: organic compounds that can form keto-enol tautomers, molecules capable of undergoing chemical equilibrium between a keto form (a ketone or an aldehyde) and an enol form (an alcohol), and includes the following: phenol compound, tocopherol compound, quinone compound, ribonucleic acid compound.

59. The composition of claim 54, wherein the complexation agent is one or more of: a charged phospholipid and includes the following: anionic phospholipid, lecithin, phosphatidylcholine, phosphatidylethanolamine, phosphatidylserine, sphingomyelin, synthetic phospholipids with positive charge, DLin-MC3-DMA.

60. The composition of claim 54, wherein the complexation agent is a charged protein that may be positive or negative and includes albumin, synthetic polypeptides, plasma proteins, alpha2-macroglobulin, fibrin, collagen.

61. The composition of claim 54, wherein the complexation agent is a ribonucleic acid comprising a biopolymer macromolecule comprising nucleotides, comprising a 5-carbon sugar, a phosphate group, and a nitrogenous base.

62. The composition of claim 54, wherein the complexation agent is a polysaccharide, long chain polymeric carbohydrates comprising monosaccharide units bound together by glycosidic linkages and includes: ringed polysaccharide molecule, cyclodextrin, clathrate.

63. The composition of claim 54, wherein the dispersal medium is capable of forming multiphasic colloidal suspension, and is selected from among four classes of hydrophobic oils: saturated fatty acid methyl esters, unsaturated fatty acid methyl esters, saturated fatty acid ethyl esters, unsaturated fatty acid ethyl esters.

64. The composition of claim 54, wherein the dispersal medium comprises a saturated fatty acid methyl esters comprising one or more of: methyl acetate, methyl propionate, methyl butyrate, methyl pentanoate, methyl hexanoate, methyl heptanoate, methyl octanoate, methyl nonanoate, methyl decanoate, methyl undecanoate, methyl dodecanoate (methyl laurate), methyl tridecanoate, methyl tetradecanoate, methyl 9(Z)-tetradecanoate, methyl pentadecanoate, methyl hexadecanoate, methyl heptadecanoate, methyl octadecanoate, methyl nonadecanoate, methyl eicosanoate, methyl heneicosanoate, methyl docosanoate, and methyl tricosanoate.

65. The composition of claim 54, wherein the dispersal medium comprises an unsaturated fatty acid methyl esters comprising one or more of: methyl 10-undecenoate, methyl 11-dodecenoate, methyl 12-tridecenoate, methyl 9(E)-tetradecanoate, methyl 10(Z)-pentadecenoate, methyl 10(E)-pentadecenoate, methyl 14-pentadecenoate, methyl 9(Z)-hexadecenoate, methyl 9(E)-hexadecenoate, methyl 6(Z)-hexadecenoate, methyl 7(Z)-hexadecenoate, methyl 11(Z)-hexadecenoate.

66. The composition of claim 54, wherein the dispersal medium comprises a saturated fatty acid ethyl esters comprising one or more of: ethyl acetate, ethyl propionate, ethyl butyrate, ethyl pentanoate, ethyl hexanoate, ethyl heptanoate, ethyl octanoate, ethyl nonanoate, ethyl decanoate, ethyl undecanoate, ethyl dodecanoate (ethyl laurate), ethyl tridecanoate, ethyl tetradecanoate, ethyl 9(Z)-tetradecanoate, ethyl pentadecanoate, ethyl hexadecanoate, ethyl heptadecanoate, ethyl octadecanoate, ethyl nonadecanoate, ethyl eicosanoate, ethyl heneicosanoate, ethyl docosanoate, ethyl tricosanoate.

67. The composition of claim 54, wherein the dispersal medium comprises an unsaturated fatty acid ethyl esters comprising one or more of: ethyl 10-undecenoate, ethyl 11-dodecenoate, ethyl 12-tridecenoate, ethyl 9(E)-tetradecanoate, ethyl 10(Z)-pentadecenoate, ethyl 10(E)-pentadecenoate, ethyl 14-pentadecenoate, ethyl 9(Z)-hexadecenoate, ethyl 9(E)-hexadecenoate, ethyl 6(Z)-hexadecenoate, ethyl 7(Z)-hexadecenoate, ethyl 11(Z)-hexadecenoate.

68. A method of treatment of mitochondrial dysfunction in a subject's eye, the method comprising:
delivering a prodrug of a mitochondrial targeted tetrapeptide combined with the extended release drug delivery system into the subject's eye at a treatment start; and
cleaving, by action of an esterase in the subject's eye, the prodrug to release the mitochondrial targeted tetrapeptide into the eye during a first phase at a burst phase release rate; and
cleaving, by action of the esterase, the prodrug to release the mitochondrial targeted tetrapeptide into the eye during a second phase at a steady-state release rate,
wherein the burst phase release rate is greater than the steady state release rate, further wherein the first phase extends from the treatment start for about 2-6 weeks and the second phase extend from an end of the first phase for one or more months.

69. The method of claim 68, wherein delivering the prodrug comprises delivering a prodrug compound of formula (I): 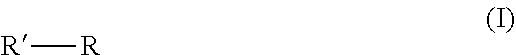
where R′ is a mitochondrial targeted tetrapeptide (MTT) containing alternating cationic and aromatic amino acid residues in which the C-terminal amino acid is covalently linked to R by a cleavable covalent bond, where R is a conjugation moiety that may be removed by enzymatic cleavage, catalysis, hydrolysis, or other reaction to yield free mitochondria targeted peptide R′ and conjugation moiety R, where R is selected from:
a C4-C30 lipid moiety,
an C4-C30 straight-chain or branched aliphatic moiety,
a 2-mer to 30-mer peptide moiety,
a pegylated moiety, or
a carbohydrate moiety.

70. The method of claim 68, wherein delivering the prodrug comprises delivering a prodrug compound of formula (II): 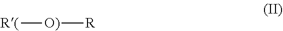
where R′ is a mitochondrial targeted tetrapeptide (MTT) containing alternating cationic and aromatic amino acid residues in which the C-terminal amino acid hydroxyl group is linked via ester bond to R, where R is a conjugation moiety selected from:
a C4-C30 lipid moiety,
an C4-C30 straight-chain or branched aliphatic moiety,
a 2-mer to 30-mer peptide moiety,
a pegylated moiety, or
a carbohydrate moiety.

71. The method of claim 68, wherein delivering the prodrug comprises delivering a compound of formula (III): 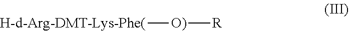
where R is a conjugation moiety selected from:
a C4-C30 lipid moiety,
an C4-C30 straight-chain or branched aliphatic moiety,
a 2-mer to 30-mer peptide moiety,
a pegylated moiety, or
a carbohydrate moiety.

72. The method of claim 68, wherein delivering the prodrug comprises delivering a compound of the formula H-d-Arg-DMT-Lys-Phe(-O)-octadecyl, also termed H-d-Arg-DMT-Lys-Phe(-O)-stearyl.

73. The method of claim 68 wherein the released mitochondrial targeted tetrapeptide drug has alternating cationic and aromatic residues.

74. The method of claim 68 in which the released mitochondrial targeted tetrapeptide drug is H-d-Arg-DMT-Lys-Phe.


SEQUENCE LISTING (635 sequences)

SEQ ID NO: 1-3 - skipped (3 entries)

SEQ ID NO: 4 - protein, 4 aa, Artificial Sequence
  Mitochondrial-targeted tetrapeptide
YKFR

SEQ ID NO: 5 - protein, 4 aa, Artificial Sequence
  Mitochondrial-targeted tetrapeptide
YKFK

SEQ ID NO: 6-33 - skipped (28 entries)

SEQ ID NO: 34 - protein, 4 aa, Artificial Sequence
  Mitochondrial-targeted tetrapeptide
FKFR

SEQ ID NO: 35 - protein, 4 aa, Artificial Sequence
  Mitochondrial-targeted tetrapeptide
FKFK

SEQ ID NO: 36-38 - skipped (3 entries)

SEQ ID NO: 39 - protein, 4 aa, Artificial Sequence
  Mitochondrial-targeted tetrapeptide
YRFR

SEQ ID NO: 40 - protein, 4 aa, Artificial Sequence
  Mitochondrial-targeted tetrapeptide
YRFK

SEQ ID NO: 41-68 - skipped (28 entries)

SEQ ID NO: 69 - protein, 4 aa, Artificial Sequence
  Mitochondrial-targeted tetrapeptide
FRFR

SEQ ID NO: 70 - protein, 4 aa, Artificial Sequence
  Mitochondrial-targeted tetrapeptide
FRFK

SEQ ID NO: 71-73 - skipped (3 entries)

SEQ ID NO: 74 - protein, 4 aa, Artificial Sequence
  Mitochondrial-targeted tetrapeptide
  MISC_FEATURE (4)..(4): D-Lysine
YRFK

SEQ ID NO: 75 - protein, 4 aa, Artificial Sequence
  Mitochondrial-targeted tetrapeptide
  MISC_FEATURE (4)..(4): D-Lysine
YKFK

SEQ ID NO: 76-103 - skipped (28 entries)

SEQ ID NO: 104 - protein, 4 aa, Artificial Sequence
  Mitochondrial-targeted tetrapeptide
  MISC_FEATURE (4)..(4): D-Lysine
FRFK

SEQ ID NO: 105 - protein, 4 aa, Artificial Sequence
  Mitochondrial-targeted tetrapeptide
  MISC_FEATURE (4)..(4): D-Lysine
FKFK

SEQ ID NO: 106-178 - skipped (73 entries)

SEQ ID NO: 179 - protein, 4 aa, Artificial Sequence
  Mitochondrial-targeted tetrapeptide
KFRY

SEQ ID NO: 180 - protein, 4 aa, Artificial Sequence
  Mitochondrial-targeted tetrapeptide
KFKY

SEQ ID NO: 181-208 - skipped (28 entries)

SEQ ID NO: 209 - protein, 4 aa, Artificial Sequence
  Mitochondrial-targeted tetrapeptide
KFRF

SEQ ID NO: 210 - protein, 4 aa, Artificial Sequence
  Mitochondrial-targeted tetrapeptide
KFKF

SEQ ID NO: 211-213 - skipped (3 entries)

SEQ ID NO: 214 - protein, 4 aa, Artificial Sequence
  Mitochondrial-targeted tetrapeptide
RFRY

SEQ ID NO: 215 - protein, 4 aa, Artificial Sequence
  Mitochondrial-targeted tetrapeptide
RFKY

SEQ ID NO: 216-243 - skipped (28 entries)

SEQ ID NO: 244 - protein, 4 aa, Artificial Sequence
  Mitochondrial-targeted tetrapeptide
RFRF

SEQ ID NO: 245 - protein, 4 aa, Artificial Sequence
  Mitochondrial-targeted tetrapeptide
RFKF

SEQ ID NO: 246-317 - skipped (72 entries)

SEQ ID NO: 318 - protein, 4 aa, Artificial Sequence
  Mitochondrial-targeted tetrapeptide
  MISC_FEATURE (2)..(2): D-Arginine
YRFK

SEQ ID NO: 319 - skipped

SEQ ID NO: 320 - protein, 4 aa, Artificial Sequence
  Mitochondrial-targeted tetrapeptide
  MISC_FEATURE (2)..(2): D-Arginine
FRFK

SEQ ID NO: 321-461 - skipped (141 entries)

SEQ ID NO: 462 - protein, 4 aa, Artificial Sequence
  Mitochondrial-targeted tetrapeptide
KYRF

SEQ ID NO: 463 - protein, 4 aa, Artificial Sequence
  Mitochondrial-targeted tetrapeptide
KYKF

SEQ ID NO: 464-496 - skipped (33 entries)

SEQ ID NO: 497 - protein, 4 aa, Artificial Sequence
  Mitochondrial-targeted tetrapeptide
RYRF

SEQ ID NO: 498 - protein, 4 aa, Artificial Sequence
  Mitochondrial-targeted tetrapeptide
RYKF

SEQ ID NO: 499-561 - skipped (63 entries)

SEQ ID NO: 562 - protein, 4 aa, Artificial Sequence
  Mitochondrial-targeted tetrapeptide
  MISC_FEATURE (3)..(3): D-Lysine
RFKF

SEQ ID NO: 563 - protein, 4 aa, Artificial Sequence
  Mitochondrial-targeted tetrapeptide
  MISC_FEATURE (3)..(3): D-Lysine
KFKF

SEQ ID NO: 564-635 - skipped (72 entries)